SEQUENCE LISTING (15 sequences)

SEQ ID NO: 1 - DNA, 7447 bp, Artificial sequence
  The sequence represents a binary vector, which comprises a T-DNA with a nos-promoter::nptII::nos-terminator cassette, which confers kanamycin resistance and a sequence stretch of “NNN ecific DNA-endonuclease
ccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcnnnnnnnnnngccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgtt

SEQ ID NO: 2 - DNA, 2835 bp, Artificial sequence
  Nucleic acid sequence coding for insert of pCB603-100
agatctagtaggaaacagctatgaccatgattacgccaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgcctgcaggtcgagcggccgccagtgtgatggatatctgcagaattcgcccttttagatcttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatatgaaacaacaatgctaaatttcgaatcttgatttgatctcttcagatccgagaaaaccctaacttccgaaaatcaccaccattaacactttacagagacaacctcaacacaaactcactcattttttcgataacccttttaaaaacccattagcattaaaacaaacaaagagtaaaaaataaaatcaaaatcctaattctccggcgttctcgtcgtcgtctttcctcaatcaaaccacactctctctcacacacacactgtaaaacaaaagttgaataaagatgaaacttttttttttaaacaaaaaaataagagctttttggggaaaatctacgtgaagtaataataacaaaagagctttttattaagccgcaacaacaagcctgccagagaaactatgattagtgtgttgttgttgttcctctctttttcttcttatgaataaaatagctttttcgttgatatattatttaatatttttaaagatctcaacgggctttatttatgggcatgcagtggtgtagaggaggaagacgaggaatataagctgcccgtaacggtgctgctgcccgaactgaccggtcaaaaaaacgggttcgggggagagaagagatggttgaaagtgacatcgctttgggtttggggtaaagtagttgaaagctcttaaggctatgcacccgtttctcctggtcaaacctctctctgttgggtcccttttttctttaagtagaaaaaccttattctaaatttttaatgcaagtaagattcgtaatcccatatttatgaaagtaaagttaaaatgttaaagaggttcactatcatgtgagcaatctcataccaaaaatgtgattttttttttacaatggttgtgaattttgtgattgataaaccatgcaacaataaagatattagtgtttgctgatattctttctaaacccaactttggcagagatgagattgaaagatttgagttttgtcaacaaaaaatttggtggaagaaactgtagagacggcaaagacaaaatttggccaagttccactgtggaagtagtatggcgtgcccgatatgactttactactgtttcatcacaataccattaaacagatacttgtcttatcatttaaaaaaaaaaaaaaactgcccatacaatttgagtagaggtaaaaagtaaaataaaaagcagattacatttgttaccctttaaatatcacacattatgcttaccccatgtggtggatggactctttattttcattatgatcaaaaggtttctagaacaaaatttaattaaagtaaaaaagaaacagtcatcttcagaaaatatttcaagataatgcttacaattatttatttagatattgtgagtggctaaatcagctgccaatgagtgaatttacaaacttttttataatccccaacattttttatattcatgataaaaatatgcttcaaattcaaaatttcacaaatcagaaataattctaaaaacaataagagctaccttgttatgaaagagaaacgaactttaagaagattttatcaaaattaaatgaaacagtagaattcaaatttgcgacaaaaaatacgcctcctaaatacaacgggataatatcctagtgcaagtggccaccgagcgtttcaactttcaaatccaggaataaaggtaagaaaaagaacaaaacaagagaaaacacagaatttacagaggaaagataccatgcaaagaaaaagaagacatggacgcaaacgccataatttgggaattctatattaagggcgaattccagcacactggcggccgttactagtggatcaattcactg

SEQ ID NO: 3 - DNA, 2187 bp, Artificial sequence
  Nucleic acid sequence coding for insert of pCB622-3
agatctagtaggaaacagctatgaccatgattacgccaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgcctgcatgctcgagcggccgccagtgtgatggatatctgcagaattcgcccttttagatcttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatatgagtgttctcttctctttctctcagactaaaatctcaggtttgtagtagaaaaagtctaagtagtgggggcgtcttctatataggaggcttcttgaggaagatgagccgttttataagaaataaaaacaaaattaaagaaaataaatgaaccccaacggtctgattgtttttgaaaaattagaggtcgtgggcctgttacgggccttacggctgtcgagacttacaaaatcgggctttttcactattgggattttgaagtcacaacggcccacttattctttttattgactgtgatataggaaaaaagaaaagaaaaaaagatttgtgtgaattagattgctagttaaatccggttatctagatgttgatttctgtttggtttacatggttttgaaataaaaatagatttttgttcggtttgcattagaattagtacggttttgagaaaaaaaaacaaaagaaaaaggtcactcgaggctgttttggaaacaaagcgagtagatgtaaaatatgcatgattttgtgggtttacccctacagaaatcgacgacacgcgctcaacgcgaggttggtgcgagtgggcgtgatctggagcgcgtgacgcgaagttggtgcgagtgggcgttttctggtgcgttctgattggctaaaaaaatgaaaatagtagtgatgctcctcctctattattttgtttttcgtcattcttcagttatgaaagttgtgtggttttaagatgcaattgagtttattctgaggttttttggttattttgaaatcattgattaatgtcacaaaaaaaaagtagaaactagattttgagatattaaaattttcattttcattttgttctgcaatcattgaaaactagattcagctgagccattgaagaaatcaaagtgagttgaatatttttctaaaaatgtgaaaattggtaaaagttggtcctcattttgttagtttaatgttttcttgactttaggagatgaactttcaactgcattgcagatcccatcacacattaatctcactctcttctgcacattgcatggtgtggtgtagatatggttcccacacaatttgtcatattcatttaccacaaagttcatgaatcatgtttgaagtgaatcctttttctttcaaatttagtcatctcactagtggatcaattcactg

SEQ ID NO: 4 - DNA, 2252 bp, Artificial sequence
  Nucleic acid sequence coding for insert of pCB653-37
agatctagtaggaaacagctatgaccatgattacgccaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgcctgcatgctcgagcggccgccagtgtgatggatatctgcagaattcgcccttttagatcttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatatgcacacacagtcttaaaacgactgcgtttttagatatactttaaaagctttcaagtctccatgaagaagaaacagaggatagagaagaagaagagaaccgtattatgtaccgacattagattttttttctttcttttattatgatttctacagaaatttgtttttgttttttcactttaaccgagaaaaaatgatttttatcgttccatcacatcaaattctggcggaaatttacagtggtttcattgagtgctcagacgacagaaatacagagtaagatagagatggagagagaaagagagagagtctttaaagagaggagattgtacaaatgtagggacctgttgtgtaagagagagagatgaagggaagaagaattatttggggaggataaagttaaaaaccccatcatcttttatctctctgccagagttctgaagctttgcagaagagagagatagagtgagtgagagggagtgattttgtttctctctcttgaagggtaaaacggtcataaaatgttgaagaagaaggagaagaagaacatgttaatggcagtggtgaataaaggaggtcgctttcgttgcccaatacttacgagtctttgtctctctgtggcttgtttcacagatgacccatatcaataaatccatcttatgggtacttttgtaattatgtatttttatatgattttagtgtaatttttttgtttgcttaacatgttcttcttcatcttcttacttcaattactaatttaaattcaggagtttacaaaaagtttggttcagttttctcttctttaattattagtatagttgttgtgtttttgtcaacgttttttagtatagtgaacgtgtgatttctaagtgtttttgtttattactaaatttagaaatatgcatgccaatgatttttggtttagcatcaacacaaaacttaataaaatatgattggttttaggatgttaaccggtttggttggctccggtcggtttgtcacaaaattaatattttgaagctttgagaacgaagtggactaaaactaaaagggtcacaggcccaacaaaagaaggaagaacttaaaaaaaataataattccttttcattttaacttctctttttttttttgttaaatcatttatttgtttcaataaaaatacacataatattatcttttctcacatagtataagggcgaattccagcacactggcggccgttactagtggatcaattcactg

SEQ ID NO: 5 - DNA, 2994 bp, Artificial sequence
  Nucleic acid sequence coding for insert of pCB652-124
agatctagtaggaaacagctatgaccatgattacgccaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgcctgcatgctcgagcggccgccagtgtgatggatatctgcagaattcgcccttttagatcttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatatggtcgactaaagggcgaattccagcacactggcggccgttactagagttttttttttttcaataattgggctttgacagatagtggtaattaatctaaataatttttttttattttttttggatagggagaaagaaacttgggaatgaagaataaatgaaaattatgaagagtatttatagtgaaaaaaatgggtagagtttgtttttgagtgaaagatggagagttaatttaggattttttttaaaaataaattaaatggattggatgattattttttatttgtttggataatatttatctaaaattttggataatttgttggttattgttacatttcatgagtctgttttactaataattttttgttagaacaagtcttttgccatttaatttgctattccgttctttaatgttttgacttttgagtgtgttaaaaaaaaaggaattcgcgaaaaataatgcaaaatttgggattttccaatttacataaataagataaaggggtaaatttgaccttaaataaaaaatgaaaaagaaaagaaaatatgatcacaaataatgtttagtggcgactaaatataatcgccacaaaaggttatcaccggtcgtggcgacttttttgagtcgccacttgaaatgttgttctttgttacgattgaggaagtagtctccacgaaggcgtaacaaaaatatgatttcttgttaccttgaaaattataagttttttccacattatttatatatggcgtgaaatttggtcatatatactatatccctagggcgaaaagctaaaagaatagtaagaaacgatattgtgcatctcttaaaattaaaaattaactatcaagtgtttttgaacagatttgattatgaatggaagtatcaacttcatcaagttactctattttagccatctttattcatttgtgattttttttttccgaggctcatcactcctcttccttctttgcttttatagttcgtcattaattaagtattcactattcttatttgtaatattatcctagagtgtgttataaattatacaataattataacttacttagttctttcgtcttctcatattttacatgcttgttaatagggatttggtgcctcaatagagttagtaaggtaaaaatttgtccttccgcttacttgcagaggcggatctacaagtatcttgagaaactagtaaaaatagagaatataaaaaagagtgcacctttagaaggcattgatctagtacgttgttggaagtaagggagtccacccctacgacctagagtcgatttcggccaacaacccattttttactcattttattacaagttaaagagtttatttgctattaaatatgacattattcgttagccgtcattaatatttttactaatgcttaaattaaaataatattgatgcacccaaactcttcaaatcctgaattcgcctctacttacttgtactcttctcaaagaacttcttattatatttgaataaaataagtcacataacaacttgcctaatattaccaaatattagttctacataaaaccacacatttattgtgtaacaaaacaaaaaggaaagctaacaaatttctttcttagtatatatatggtactagacaagtccatatacacactctttgccttctaaattcaccaaaataaattaagagacaattatagaggattcttgaggacatcatctactgttaaatttgacacaatttttgtctctttagacatatatggttgtagacataattcctttttaaaaaaaaatatttagtgcatcttaggtacatccatgctccaagcattaagagtctcaattgtgattgtctcagatccattattgaagacaaataaatgtgcattattatgaatcgctaacgttggatacaccctcgatgttatgcatgtaagggcgaattccagcacactggcggccgttactagtggatcaattcactg

SEQ ID NO: 6 - DNA, 2586 bp, Artificial sequence
  Nucleic acid sequence coding for insert of pCB632-17
agatctagtaggaaacagctatgaccatgattacgccaagcttgcatgcctgcaggtcgactctagaggatccccaagtcgacggatcctgatagcttatactcaaattcaacaagttatatataaatgtatagatactacaatatcattaacaaaagtcaccttaaataaatacacatatcttttatgttctctattgttttgcgtacgctaacacaatttctcatatgcaaaaggatgaatgagtaacaaattacctcataagaacaatcatctttgcttacatactaatacaataatcactcaatcaaccaataacatcaatcacataggtttacatacaataatcactcaatcaacttcataagaagaatcatgtttacttaattcatcaattatccccaaaaacaccactattaagtataaactataacatatttgtagtgatgggtcaacatttttatcatatttaaactcgggttccctcaaatcgagaaatagtgaacatgtaatattaattttaaatcgcaattacagaaattaattgaatttggtcaaatggacagaattttatagattgggtggaactagaaaaaaaaaaaaaaagagtatagggtgaattgagtacatgaaagtacatggtaatcctagttaaacgcataatacatgtgggttcatttgtatttttttgtaacttacgagtaaactggctacaacaaaaaaaattagaagatttttttgttttgtagaaaaccctaattttagttatagttgtataactttgataaaattataaaattgtattacgaaaaaagtaataagatattcaaaaaagcctagaataacgtatatgactatgagcatgaaactgcaagtcaaatgctgacagacaaccataaacaaaagaaattaaatagagatacctttaaaataagtaaaatttcatttataaaaaatctactttcttgtgaatctgtcacgttcaataatttgaagaccactcaacatacaaggtaaataatgaaaaataaaatctaccaaaatttcaatcattattatcttccaaaaaaacaaaattatacagatgatgatggtgatatggaacttcgattggctaatattcactgtgtctctaaaaaccatccacttatcaagataagatggaccctacactcatccaatctaaaccagtatctcaagattcttatctaattacatcattctctaccgttagatgaaattgaccattaaccctaccataactccatacaccgcgagatactggattaaccaaatcgagatcatcgtagccgtccgatcaacaagtaccatctcttgaaatactcgaaatcctcataagtccgtccctctttgctctcactatcaaaactctgaatttcgatttcatctagagtcgacgtctgggtaccgagctcggatccactagtaacggccgccagtgtgctggaattcgccctttagtcgaccatatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctagcctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataagatctaaaagggcgaattctgcagatatccatcacactggcggccgctcgagccgaatcactagtctagagtcgaccgtaccccctgcagatagactatactatgttttagcctgcctgctggctagctactatgttatgttatgttgtaaaataaacacctgctaaggtatatctatctatattttagcatggctttctcaataaattgtctttccttatcgtttactatcttatacctaataatgaaataataatatcacatatgaggaacggggcaggtttaggcatatatatacgagtgtagggcggagtgggggggatcggcatgcaagctagcttggcgtaatcatggtcatagctgtttcctactagatctgaattcactg

SEQ ID NO: 7 - protein, 235 aa, Artificial sequence
  Amino acid sequence coding for I-SceI
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLASLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 8 - DNA, 982 bp, Petroselinum crispum
gaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

SEQ ID NO: 9 - DNA, 12862 bp, Artificial sequence
  Nucleic acid sequence coding for binary vector pCB666-3
aattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctctcagtatttaatccggccatctccttccgttatgacatcgttgaaagtgccaccattcgggatcatcggcaacacatgttcttggtgcggacaaatcacatccaacaggtaaggtcctggtgtatccagcattgtctgaatagcttctcggagatctgctttctttgtcaccctcgccgctggaatcccgcaagctgctgcaaacagcaacatgttcgggaatatctcgtcctcctgagccggatctccgagaaatgtgtgtgctcggttagctttgtagaaccgatcttcccattgcataaccatgccaagatgctggttgtttaataaaagtaccttcactggaagattctctacacgaatagtggctagctcttgcacattcattataaaacttccatctccgtcaatatccacaactatcgcatcagggttagcaacagacgctccaatcgcagcaggaagtccaaatcccatagctccaaggcctcctgatgatagccactgccttggtttcttgtaattgtagaactgcgccgcccacatttgatgttgcccgacaccagtacttattatggcttttccatcagtcaactcatcaaggaccttaatcgcatactgtggaggaatagcttccccaaacgtcttaaagctcaacggaaacttctgtttctgtacgttcaactcattcctccaaactccaaaatcaagtttaagctcctccgctcggttctcaagaaccttattcatcccttgcaaagccagcttaacatcaccacacacagacacatgaggagtcttattcttcccaatctcagccgagtcaatatcaatatgaacaatcttagccctactagcaaaagcctcaagtttacccgtgacacgatcatcaaaccttaccccaaacgccaacaacaaatcactatgctccacagcgtaatttgcatacacagtcccatgcattccaagcatatgtaacgacaactcatcatcacaaggataagatcccagccccatcaacgtactcgcaacagggattcccgtaagctcaacaaacctacccaattcatcgctagagttcaaacaaccaccaccaacatacaacacaggcttcttagactcagaaatcaacctaacaatctgctccaaatgagaatcttccggaggtttaggcatcctagacatataaccaggtaatctcatagcctgttcccaattaggaatcgcaagctgttgttgaatatctttaggaacatcaaccaaaacaggtccaggtctaccagaagtagctaaaaagaaagcctcctcaataatcctaggaatatcttcaacatccatcacaagatagttatgcttcgtaatcgaacgcgttacctcaacaatcggagtctcttgaaacgcatctgtaccaatcatacgacgagggacttgtcctgtgattgctacaagaggaacactatctaacaacgcatcggctaatccgctaacgagatttgtagctccgggacctgaagtggctatacagatacctggtttacctgaggatcgagcgtatccttctgctgcgaatacacctccttgttcgtgacgaggaaggacgttacggattgaggaagagcgggttaaggcttggtgaatctccattgatgcacctccagggtaagcgaatacggtttctacgccttgacgttctaaagcctcgacgagaatatcagcgcctttgcggggttgatctggagcgaatcgggagatgaatgtttcgggtttggtaggtttggttggagagggagtggttgtgacattggtggttgtgttgagcacggcggagatggaggagggagagctggatttgataccgcggcggcgggaggaggaggatgatttgttggggtttagggagaatgggagggagaatctggagattggtaatggtgatttggaggaggaaggagatggtttggtggagaaggagatcgaagaagatgttgttgttgttgttgttgccgccgccatggtacccagacgtcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatcctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagctgcgatcgccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcctgcaggttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaggtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatggcgcgccggatccgggctgcacatacataacatatcaagatcagaacacacatatacacacacaaatacaatcaagtcaacaactccaaaaagtccagatctacatatatacatacgtaaataacaaaatcatgtaaataatcacaatcatgtaatccagatctatgcacatatatatatacacaattaataaaaaaaatgatataacagatctatatctatgtatgtaacaacacaatcagatgagagaagtgatgttttcagatctgtatacatacaaacacaaacagatgaacaattgatacgtagatccatatgtatacgtacaattagctacacgattaaatgaaaaaaatcaacgatttcggattggtacacacaaacgcaacaatatgaagaaattcatatctgattagatataaacataaccacgtgtagatacacagtcaaatcaacaaatttatagcttctaaacggatgagatgaacaagataaagatattcacataaggcatacataagataagcagattaacaaactagcaataatacatacctaattaaaacaaggaataacagagagagagagagagagagagatttaccttgaaaatgaagaggagaagagaggatttcttaaaattgggggtagagaaagaaagatgatgaattgtgagaaaggagagatagaagggggggttgtatatataggctgtagaagattatttttgtgtttgaggcggtgaaggaagaggggatctgactatgacacgtttgcggttacgtatttcgataggagtctttcaacgcttaacgccgttactctatatgaccgtttgggccgtaacggggccgtttgttaacgctgatgttgattcttttctttctttctttcttccttttttaaagaagcaattgtacaatcgttgctagctgtcaaacggataattcggatacggatatgcctatattcatatccgtaatttttggattcgaattctctagtcaagcttacggtctagatattgtttttgtttcacataaatgtcgttttggattattcatgtaatattttaaactaaagtacaatttttgactactttagtttactagttaagcttttatttttttgactaaccattgaatgatgaagagatcaacgcatcatatttacaacttacatagtcttttggaagtgtaaattgctaatactacctaaaatatatctataattaactaatattttttcgtcaattataatagatcaattaaaaggctatcaaaaggaaaaaaatgaaatccacatcctgccatcataacctcatgctggaaaaagaaatgaaaaaatataaaaaatttcttttgtttattaaatttacaactttaatactagtttcttttctattttttaaaagcttttgtcacttacttaaaaaaaaaaaactttttgaaatattcctacttccaatgtctgattagtgcttctggatttcctttttggatcatgtgaatcctaaatcagaaaaattcatataatacccaattcagtatattttcatacttcaatttacaagagttctctatgtttttagcttctttcttttaagccaaatgttttaagcatcttttatacattaaaataatttagtgttgagttgagattttttttttttttttttggatttacttgttcaaaatctgaaaaaatgtttacagaaggttaaaatgaaccaaaaggcatatcaagctagattttgaattaccctatttcatcgtatacacaaaactgataatgtggacacagttgattttacttctcgatgacatcgtagttttatttaatttggaaaccacggcccatatgagcacatttcaattaaaaaccaatggtaagagcattttccatgcaagattcgagagatattaacccagtgactgttaaaacagcttagaaccctaataacgaatttcaattactcaatttaccattcgcatttcgcaataaccaaactgagccagtcacaaggagtaaaccgaaccggattatttatttataaaatgaaagaaaggaaaccaaacaacaacagcagtagtagtctgacgtaaaccaaaaagcaggcagatcaacaactaaaagaaactcaaattaccaaaacaaacaggaaattgcaaactaagtttttttaccatatgcatacaaagaccataaaaggttctgataatcaccgggctcgaggtaccgagctcgaattcactggccgtcgttttacaacgactcag

SEQ ID NO: 10 - DNA, 11824 bp, Artificial sequence
  Nucleic acid sequence coding for binary vector pCB657-41
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccccaaactgtctcacgacgttttgaacccagattaccctgttatccctagtcgactctagactagtggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatccgatatcgcccgggctcgagtctttgttttttactttggttcatgacactcagagacttgagagaagcaatatatagacttttttttgttttttttttgtggtcacgtttattttcctattggagacggtaacgaagatcgaacctgtggtggaaatgaaacaaggtgggactagcccacgtggtttcttttctctgcattgatttgtttttgttttttttgtaaagttcacatcaaacctactaataattgagaagaaaaataaaatctattgattgattaaaccagccgatgctttatgtctgaatataaaaaagaagtgaaaaccccgtttaagaattacaacggtggtttacaaagtatttggacacaataaatccaaacgaaataaaacaaaatggagaactaccaaataaaaaacaaataaaaaacttaaaagaatttattccattttttttcccgtagaatttattcttttatggattccttaaatccatatttgatgcattttgattcctcataataggtaataatatatactatgttatagatatgtttctaattcgtattaacctacctttttttggtcgtacgattctacctaataatattgaacggaattgatgttttggaccacttagaaagtattttttttttggtttgtcttagctgtatttcattaaatataaatttaaataagaaatgtcataaataaaatttgacgtatagattttttaaatccattttatgttatttaatatttgaaatgtgagtttggctcctatttaatcttaggatgggttaatactaagttttccttaatgaattatctcagagaaactggattaaataaactaaaaaatagatcaatgtgttttggtccggtcaaatatctttggatttactattattggcgaaaagaaagtctcatatagtaaatcatattcctacaagagaaatcaaaatttttgaattaacatggattgtatagtttcttatataaccaattagttcgcatcaagaaaaccaaaccccaattaataatcaaacgggcttggtaggaatatttcattgcagctttcagataaaagaaaaaaacacacactcaagtcttttatttcatctttcttacttgcaggaactcaaattccactttgccacttttctttacaaataaacacaaattgtcaatgaaacgaaatagtctttttatgcaaacactgtttgtcttttttcgatcacgtttctgattgtgacagccatccatatatatagggaatgtaaaacaacaacatgtgaagtcacatatacgtaatggtttagcatagcttctattttcgttgtcaatattagtcattccaaaacatttttaagaaaaataaattaatatatgtatattcttggaactaatgtatgtggaaatacagtaacttaattattaaacattctaaatgcaaatatgcaaagaaaaaaaagaaaagaacacaactgaaatcaaagccagattcataataattggctacatggttgtagaatgtagggtaacacaacatccagaattgaacactcaaattggatgatagatggataatctttagatacaagagaattggttctcttccattattaacgaaaataaagaaaaaaagtttagcataaaagtttgaaactcaacataacattttgaacttgactccttcataggagtgacatgaactgacgaatcacaaccgattacttgtttgagtcatcttccgctttctccaccttcgaaatgaatgtgaccggtttcttcgggtgctcatttacggtcaagtgtaaaacatctggtctcgaggtacctggtagggataacagggtaatctgggttcaaaacgtcgtgagacagtttggtgcaggtcgaaattcgagctcggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgaccaattccacagttttcgcgatccagactgaatgcccacaggccgtcgagttttttgatttcacgggttggggtttctacaggacgtaccatggtcgatcgactctagactagtggatccgatatcgcccgggctcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatccgggggatcctctagagtcgaggtaccgagctcgaattcactggccgtcgttttacaacgactcagtactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 11 - DNA, 13531 bp, Artificial sequence
  Nucleic acid sequence coding for binary vector JB010qcz
gtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtggtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcaggatcccagtctagattatgtatttccaactttcattaacaatataatcgcatataaatgaaaaatcgtttccaggataatattttgatgaaatctcatattattgttcgtactcggattgatgttgaaggcttgaagcgcttcaaattatagaccagattatttaagtttttcttttgtttactccatatcaatttgatccattatactacctaagaaaatttaggtaacatagaattatttattgttatagtaaaaaaaaggaaaaccacaaaaataatctacttttacgtatatactattttcatgacataagtaattaagttgtacaacttttttttaatgaaaagagagagtaaatttatcatgttcatgtgtagttacctcgtgaataaccgacggttatatagacgcctaacatgaattgttcagttgaagacagttcaaaacatgtgtttcactctaaaatcctcaacaaaaaaaaagtgttaaaatttgtaaacctctttcaagcaaaaaaagaaaaagtgttagaatcccaagattctttcataatccggaatcttggctgaaaacgtataaaagagattgacgtagtaacaaggagtcttggtatgcttccatgctttttatccttttttgtcatggaaccatgatttggttaccatttattatgtaaccgaaattttcattgtaataatgaatatttaaatttttagcaaaaaaaaacaaaaaaaaacaaggagtcttgtcttcgttctcaaatttcagagctcttgcacttttcaagagttttactttgatgagtgagacatttgtctttttagtgtttattttctaaacttaaaatagtagcatcaacatcactcaattataattcttaagatgttgtagaaaaatattttatagatggaaagtaatcgatattaagacaaataagaaaccaaaccggactttgtgttcagaccgaatcaaatctgaattggagaaattatggtggaggcgaaagtcaacggaactaaagtataaaaccaaatgtcaaaaataaaacccaattttcatccttaaacgaacctgctgaaaccctaatttcgattaccaattccgatctaaaaagaagtcatggaagccattgattccgcaatcgatcctctcagagatttcgctaagagcagtgttcgtctcgtccagcgctgtcacaaacccgatcgcaagggtaacgccttttctcaaaaaaatctcatttccgatttttgatctgtagattagggttttctgaaattttgatatcatttgtaattgaattggttatcagaattcacgaaagtagctgtgcgtacggcgattggatttgtggtgatgggattcgttggattcttcgtgaagctcgttttcatcccaatcaacaacatcatcgttggatcttcttagtgtagtactttctttacgaggtaattgatctcgcattatatatctacattttggttatgttacttgacatatagtcattgattcaatagttctgttaattcctttaaagatcattttgactagaccacattcttggttcattcctcaataatttgtaatcatattggtggatatagaagtagattggttatagatcagatagtggaagactttaggatgaatttcagctagtttttttttttggcttattgtctcaaaagattagtgctttgctgtctccattgcttctgctatcgacacgcttctgtctccttgtatctttattatatctattcgtcccatgagttttgtttgttctgtattcgttcgctctggtgtcatggatggagtctctgttccatgtttctgtaatgcatgttgggttgtttcatgcaagaaatgctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctttaaaatctaaatctcaaatcaccaggactatgtttttgtccaattcgtggaaccaacttgcagcttgtatccattctcttaaccaataaaaaaagaaagaaagatcaatttgataaatttctcagccacaaattctacatttaggttttagcatatcgaaggctcaatcacaaatacaatagatagactagagattccagcgtcacgtgagttttatctataaataaaggaccaaaaatcaaatcccgagggcattttcgtaatccaacataaaacccttaaacttcaagtctcatttttaaacaaatcatgttcacaagtctcttcttcttctctgtttctctatctcttgctcatctttctcctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagaccgtaagcttgactagagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcccggatccggcgcgccatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagaccttcaaacaccaagctttcaacaaactggctagcctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataacctgcaggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgatcgcagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatgtgttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagg

SEQ ID NO: 12 - DNA, 10765 bp, Artificial sequence
  Nucleic acid sequence coding for binary vector pCB583-40
ttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagcttattccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagtccggcaacaggattcaatcttaagaaactttattgccaaatgtttgaacgatcggggaaattcgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgcttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcggtccagactgaatgcccacaggccgtcgagttttttgatttcacgggttggggtttctacaggactctagctggcacagagttaccgggtgaatttcgctaccttaggatcgtaaccaatatgtctcacggcgttttcggactagactattaccctgttatccctaggcggccgctggcaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggatgtagatcgaattgggattggtgacgttcgtcacctgcttagtctgagtctgagtcttcagaagtagatgatcccggggatccatcactttcttctgtagaggtttcttcttcctcttctttgtattctaggtacgtaccctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactcggtaccgagctcgaattcactggccgtcgttttacaacgactcagccagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatcggtcgatcgactcagatctgggtaactggcctaactggccttggaggagctggcaactcaaaatccctttgccaaaaaccaacatcatgccatccaccatgcttgtatccagccgcgcgcaatgtaccccgcgctgtgtatcccaaagcctcatgcaacctaacagatggatcgtttggaaggcctataacagcaaccacagacttaaaaccttgcgcctccatagacttaagcaaatgtgtgtacaatgtagatcctaggcccaacctttgatgcctatgtgacacgtaaacagtactctcaactgtccaatcgtaagcgttcctagccttccagggcccagcgtaagcaataccagccacaacaccctcaacctcagcaaccaaccaagggtatctatcttgcaacctctctaggtcatcaatccactcttgtggtgtttgtggctctgtcctaaagttcactgtagacgtctcaatgtaatggttaacgatgtcacaaaccgcggccatatcggctgctgtagctggcctaatctcaactggtctcctctccggagacatgtcgagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcg

SEQ ID NO: 13 - DNA, 9103 bp, Artificial sequence
  Nucleic acid sequence coding for binary vector pRS8
ttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagcttgcatgcctgcaggtcgactctagactagtggatccgatatcgcccgggctcgagagtgccaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgcctgcaggtcgactctagaggatccagtactcgacttattatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatcacttcttctgtagaggtttcttcttcctcttctttgtattctaggtacagtactggatccccgggtaccctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactccatgggaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcg

SEQ ID NO: 14 - DNA, 897 bp, Artificial sequence
  I-SceI coding sequence comprising the potato PIV2 intron at position 565 to 753
atgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctagcctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataa

SEQ ID NO: 15 - DNA, 983 bp, Petroselinum crispum
  promoter (1)..(983): parsley ubiquitin promoter (alternative sequence)
gaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcag

(19) United States
(12) Patent Application Publication
(10) Pub. No.: US 2008/0134351 A1
(43) Pub. Date: Jun. 5, 2008

(54) Recombination Cassettes and Methods For Sequence Excision in Plants

(75) Inventors: Rocio Sanchez-Fernandez, Quedlinburg (DE); Christian Biesgen, Quedlinburg (DE); Michael Leps, Halberstadt (DE); Jeffrey A. Brown, Apex, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 11/663,486

(22) PCT Filed: Sep. 17, 2005
(86) PCT No.: PCT/EP05/10058

§ 371 Date: Mar. 22, 2007

Related U.S. Application Data
(60) Provisional application No. 60/612,432, filed on Sep. 23, 2004.

(51) Int. Cl. A01H 1/06; C07H 21/04; C12N 15/63; A01K 67/00; C12N 5/10; C12N 5/00; A01H 5/00

(52) U.S. Cl. 800/13; 800/267; 536/23.2; 435/320.1; 435/419; 435/325; 800/298

(57) ABSTRACT

The invention relates to improved recombination systems and methods for eliminating maker sequences from the genome of plants. Particularly the invention is based on use of an expression cassette comprising the parsley ubiquitin promoter, and operably linked thereto a nucleic acid sequence coding for a sequence specific DNA-endonuclease.

### FIELD OF THE INVENTION

The invention relates to improved recombination systems and methods for eliminating maker sequences from the genome of plants.

### BACKGROUND OF THE INVENTION

An aim of plant biotechnology is the generation of plants with advantageous novel characteristics, for example for increasing agricultural productivity, improving the quality in foodstuffs or for the production of certain chemicals or pharmaceuticals (Dunwell J M (2000) J Exp Bot 51:487-96). Transformation of plants typically involves the introduction of a gene of interest (“trait gene”) and a marker sequence (for example a selectable marker such as a herbicide resistance gene) into the organism. The marker sequence is useful during the transformation process to select for, and identify, transformed organisms, but typically provides no useful function once the transformed organism has been identified and contributes substantially to the lack of acceptance of these “gene food” products among consumers. In consequence, there are multiple attempts to develop techniques by means of which marker sequences can be excised from plant genome (Ow D W and Medberry S L (1995) Crit. Rev in Plant Sci 14:239-261).

The person skilled in the art is familiar with a variety of systems for the site-directed removal of recombinantly introduced nucleic acid sequences. They are based on the use of sequence specific recombinases and two recognition sequences of said recombinases which flank the sequence to be removed. The effect of the recombinase on this construct brings about the excision of the flanked sequence, one of the recognition sequences remaining in the genome of the organism. Various sequence-specific recombination systems are described, such as the Cre/lox system of the bacteriophage P1 (Dale E C and Ow D W (1991) Proc Natl Acad Sci USA 88:10558-10562; Russell S H et al. (1992) Mol Gen Genet. 234: 49-59; Osborne B I et al. (1995) Plant J. 7, 687-701), the yeast FLP/FRT system (Kilby N J et al. (1995) Plant J 8:637-652; Lyznik L A et al. (1996) Nucleic Acids Res 24:3784-3789), the Mu phage Gin recombinase, the E. coli Pin recombinase or the R/RS system of the plasmid pSR1 (Onouchi H et al./(1995) Mol Gen Genet. 247:653-660; Sugita K et al. (2000) Plant J. 22:461-469). A disadvantage of the sequence-specific recombination systems is the reversibility of the reaction, that is to say an equilibrium exists between excision and integration of the marker sequence in question. This frequently brings about unwanted mutations by multiple consecutive insertions and excisions. This not only applies to the Cre-lox system, but also to the other sequence-specific recombinases (see above). A further disadvantage is the fact that one of the recognition sequences of the recombinase remains in the genome, which is thus modified: The remaining recognition sequence excludes a further use of the recombination system, for example for a second genetic modification, since interactions with the subsequently introduced recognition sequences cannot be ruled out. Substantial chromosomal rearrangements or deletions may result.

Zubko et al. describe a system for the deletion of nucleic acid sequences from the tobacco genome, where the sequence to be deleted is flanked by two 352 bp attP recognition sequences from the bacteriophage Lambda. Deletion of the flanked region takes place independently of the expression of helper proteins in two out of eleven trans-genic tobacco lines by spontaneous intrachromosomal recombination between the attP recognition regions. The disadvantage of this method is that recombination, or deletion, cannot be induced specifically at a particular point in time, but takes place spontaneously. The fact that the method worked only in a small number of lines suggests that the integration locus in the cases in question tends to be unstable (Puchta H (2000) Trends in Plant Sci 5:273-274).

WO 02/29071 discloses a method for conditional excision of transgenic sequences from the genome of a transgenic organism. Excision occurs directly by action of an enzyme (e.g., a recombinase or a endonuclease) but not via homologous recombination of flanking sequences. The recombination mechanism mediated by recombinases differs from the mechanism leading to homologous recombination between homologous sequences. It is the purpose of the method to prevent occurrence of the trans-genic sequence in the agricultural product but to have it remaining in other plant parts. In consequence, promoters employed here are inducible, seed or fruit specific promoters.

Self-excising constructs based on a site-specific recombinase are described in WO97/037012 and WO02/10415. Here also no homologous recombination but recombinase mediated recombination occurs and the recombinase recognition sequence remains in the genome making further applications of the system impossible (as described above as a general disadvantage for recombinase systems).

Several constitutive promoters in plants are known. Most of them are derived from viral or bacterial sources such as the nopaline synthase (nos) promoter (Shaw et al. (1984) Nucleic Acids Res. 12 (20): 7831-7846), the mannopine synthase (mas) promoter (Comai et al. (1990) Plant Mol Biol 15(3):373-381), or the octopine synthase (ocs) promoter (Leisner and Gelvin (1988) Proc Natl Acad Sci USA 85 (5):2553-2557) from Agrobacterium tumefaciens or the CaMV35S promote from the Cauliflower Mosaic Virus (U.S. Pat. No. 5,352,605). The latter was most frequently used in constitutive expression of transgenes in plants (Odell et al. (1985) Nature 313:810-812; Battraw and Hall (1990) Plant Mol Biol 15:527-538; Benfey et al. (1990) EMBO J. 9(69):1677-1684; U.S. Pat. No. 5,612,472). However, the CaMV 35S promoter demonstrates variability not only in different plant species but also in different plant tissues (Atanassova et al. (1998) Plant Mol Biol 37:275-85; Battraw and Hall (1990) Plant Mol Biol 15:527-538; Holtorf et al. (1995) Plant Mol Biol 29:637-646; Jefferson et al. (1987) EMBO J. 6:3901-3907). An additional disadvantage is an interference of the transcription regulating activity of the 35S promoter with wild-type CaMV virus (Al-Kaff et al. (2000) Nature Biotechnology 18:995-99). Another viral promoter for constitutive expression is the Sugarcane bacilliform badnavirus (ScBV) promoter (Schenk et al. (1999) Plant Mol Biol 39 (6):1221-1230).

Several plant constitutive promoters are described such as the ubiquitin promoter from Arabidopsis thaliana (Callis et al. (1990) J Biol Chem 265:12486-12493; Holtorf S et al. (1995) Plant Mol Biol 29:637-747), which—however—is reported to be unable to regulate expression of selection markers (WO03102198), or two maize ubiquitin promoter (Ubi-1 and Ubi-2; U.S. Pat. No. 5,510,474; U.S. Pat. No. 6,020,190; U.S. Pat. No. 6,054,574), which beside a constitutive expression profile demonstrate a heat-shock induction (Christensen et al. (1992) Plant. Mol. Biol. 18(4):675-689). A comparison of specificity and expression level of the CaMV 35S, the barley thionine promoter, and the Arabidopsis ubiquitin promoter based on stably transformed Arabidopsis plants demonstrates a high expression rate for the CaMV 35S promoter, while the thionine promoter was inactive in most lines and the ubi1 promoter from Arabisopsis resulted only in moderate expression activity (Holtorf et al. (1995) Plant Mol Biol 29 (4):637-6469).

While the maize Ubi-1 promoter demonstrates acceptable expression activity in maize and other monocotyledonous plants, expression is low (10%) in dicotyledonous tobacco plants in comparison to the 35S CaMV promoter, which makes the promoter unsuitable for most applications in dicots. Ubiquitines are ubiquitous proteins found in all eukaryotes analyzed so far. The genes for parsley (Petroselinum crispum) are described (Kawalleck et al. (1993) Plant Mol Biol 21; 673-684. Furthermore the promoter of the parsley ubiquitin gene was analyzed and described as a constitutive promoter (WO 03/102198). Other constitutive promoters are the rice atin 1(Actl) promoter (McElroy et al., (1991) Mol Gen Genet. 231:150-1609), and the S-adenosyl-L-methionine synthetase promoter (WO 00/37662). The latter is however dependant on the methionine concentration.

WO 03/004659 describes a recombination system based on homologous recombination between two homologous sequences induced by action of a sequence specific double-strand break inducing enzyme, preferably a meganuclease (homing-endonuclease). Although general statements are made about the preferable use of inter alia homing-endonucleases and the potential use of inter alia tissue specific promoters, there is no specific teaching suggesting the specific combination of features of the invention disclosed herein. European Patent Applications Appl. No. 03028884.9 and 03028885.6 describe various combination of homing endonucleases with promoters having activity in reproductive tissues.

Although these inventions solve some problems, still the extent of excision is low and the generation of homogenous, non-chimeric plants (i.e., plants in which the sequence was deleted from all cells) is time and labor intensive. The reason is mainly an non-homogeneous or insufficient expression of the endonuclease, which is needed for induction of site-specific double-strand breaks to induce homologous recombination between directed repeats flanking the sequences to be deleted. For example for the strong 35S CaMV recombination could only be observed in less than 10% of the plant cells. Such insufficient or non-homogeneous expression results in plants which are mosaic or chimeric plants (i.e., plants which comprise both cells which have undergone recombination and sequence excision and cells which have not). This requires additional plant generations (either by sexual or asexual propagation). The related efforts highly depend on the frequency of cells which have undergone homologous recombination.

It is an object of the present invention to develop systems and methods which enable the easy-to-use, highly-efficient, predictable elimination of sequences, preferably marker sequences, from the genome of a plant and allow the repeated, successive application to the same organism. This has been achieved by the present invention.

### SUMMARY OF THE INVENTION

Accordingly a first embodiment of the invention relates to a method for producing a transgenic plant comprising:
i) crossing a first transgenic plant comprising in its genome a DNA construct comprising
   a1) at least one recognition sequence of at least 10 base pairs for the site-directed induction of DNA double-strand breaks by a sequence specific DNA-endonuclease and
   b1) a nucleic acid sequence to be excised,
   wherein said elements a1) and b1) and optionally further elements are flanked by homology sequences A and A′, having sufficient length and sufficient homology in order to ensure homologous recombination between A and A′, and having an orientation which—upon recombination between A and A′—will lead to an excision of said elements a1) and b1), and
   c1) at least one additional sequence conferring to said plant an agronomically valuable trait, wherein said sequence is not localized between the homology sequences A and A′ and would not be excised from the genome upon recombination between A and A′
   with a second transgenic plant comprising in its genome an expression cassette comprising
   a2) the parsley ubiquitin promoter, and operably linked thereto
   b2) a nucleic acid sequence coding for a sequence specific DNA-endonuclease having a sequence specificity for said recognition sequence a1),
ii) generating descendants (F1) following this crossing, and—optionally—sexually or asexually generating further descendants, and
iii) isolating descendants which have undergone recombination between the homology sequences A and A′ and which do not comprise in their genome said elements a1) and b1) but comprise sequence c1).

Preferably the element b1) is an expression cassette for a marker sequence, more preferably selected from the group consisting of negative selection marker, counter selection marker, positive selection marker, and reporter genes.

In an preferred embodiment, the method further comprises the step of segregating the expression cassette for the endonuclease from the sequence c1) for the agronomically valuable trait and isolating plants comprising sequence c1) but not said expression cassette for the endonuclease.

Preferably the parsley ubiquitin promoter comprises a sequence described by SEQ ID NO: 8 or 15 or a functional equivalent or functional equivalent fragment thereof.

Preferably the orientation of the homology sequences is in the form of direct repeats, which are flanking elements a1) and b1) and optionally further elements.

Preferably the recombination mechanism between A and A′ is homologous recombination.

The sequence specific DNA-endonuclease is preferably a homing endonuclease, more preferably selected from the group consisting of I-SceI, I-CpaI, I-CpaII, I-CreI and I-ChuI.

In an preferred embodiment the construct employed in the method of the invention comprises two recognition sequences a1) which are localized between the homology sequences A and A′ and are flanking element b1) and optionally further elements in a way that cleavage at this two recognition sequences excises said element b1). The homology sequences A and A′ are preferably part of the expression cassette comprised in the DNA construct.

In an preferred embodiment of the invention, the method is employed to generate marker-free plants, thus preferably the resulting plant is selection marker-free.

Another embodiment of the invention relates to a transgenic expression cassette comprising a sequence coding for a sequence specific DNA-endonuclease operably linked to the parsely ubiquitin promoter. The endonuclease is preferably a homing endonuclease, more preferably selected from the group consisting of I-SceI, I-CpaI, I-CpaII, I-CreI and I-ChuI. The parsley ubiquitin promoter preferably comprises a sequence described by SEQ ID NO: 8 or 15 or a functional equivalent or functional equivalent fragment thereof. Other embodiments of the invention relate to transgenic vectors comprising a expression cassette of the invention, and transgenic cells or non-human organisms, preferably plant or plant cells, comprising a expression cassette or a vector of the invention. Preferably the expression cassette is comprised in the genome of the plant or plant cell.

### GENERAL DEFINITIONS

The teachings, methods, sequences etc. employed and described in the international patent application WO 03/004659 are hereby incorporated by reference.

“Agronomically valuable trait” includes any phenotype in a plant organism that is useful or advantageous for food production or food products, including plant parts and plant products. Non-food agricultural products such as paper, etc. are also included. A partial list of agronomically valuable traits includes pest resistance, vigor, development time (time to harvest), enhanced nutrient content, novel growth patterns, flavors or colors, salt, heat, drought and cold tolerance, and the like. Preferably, agronomically valuable traits do not include marker sequences (e.g., selectable marker such as herbicide or antibiotic resistance genes used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, glucuronidase, chloramphenicol acetyl transferase (CAT, etc.). Such agronomically valuable important traits may include improvement of pest resistance (e.g., Melchers et al. (2000) Curr Opin Plant Biol 3(2):147-52), vigor, development time (time to harvest), enhanced nutrient content, novel growth patterns, flavors or colors, salt, heat, drought, and cold tolerance (e.g., Sakamoto et al. (2000) J Exp Bot 51(342):81-8; Saijo et al. (2000) Plant J 23(3): 319-327; Yeo et al. (2000) Mol Cells 10(3):263-8; Cushman et al. (2000) Curr Opin Plant Biol 3(2):117-24), and the like. Those of skill will recognize that there are numerous polynucleotides from which to choose to confer these and other agronomically valuable traits.

The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. A “polynucleotide construct” refers to a nucleic acid at least partly created by recombinant methods.

The term “promoter” refers to regions or sequences located upstream and/or down-stream from the start of transcription and which are involved in recognition and binding of RNA polymerase and other proteins to initiate transcription.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Transgene”, “transgenic” or “recombinant” refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples above.

A polynucleotide “exogenous to” an individual organism is a polynucleotide which is introduced into the organism by any means other than by a sexual cross. The term “expression cassette”—for example when referring to the expression cassette for the sequence specific DNA—endonuclease—means those constructions in which the DNA to be expressed is linked operably to at least one genetic control element which enables or regulates its expression (i.e. transcription and/or translation). Here, expression may be for example stable or transient, constitutive or inducible.

The terms “operable linkage” or “operably linked” are generally understood as meaning an arrangement in which a genetic control sequence is capable of exerting its function with regard to a nucleic acid sequence, for example while encoding a sequence specific DNA-endonuclease. Function, in this context, may mean for example control of the expression, i.e. transcription and/or translation, of the nucleic acid sequence, for example one encoding a sequence specific DNA-endonuclease. Control, in this context, encompasses for example initiating, increasing, governing or suppressing the expression, i.e. transcription and, if appropriate, translation. Controlling, in turn, may be, for example, tissue- and/or time-specific. It may also be inducible, for example by certain chemicals, stress, pathogens and the like. Preferably, operable linkage is understood as meaning for example the sequential arrangement of a promoter, of the nucleic acid sequence to be expressed—for example one encoding a sequence specific DNA-endonuclease—and, if appropriate, further regulatory elements such as, for example, a terminator, in such a way that each of the regulatory elements can fulfil its function when the nucleic acid sequence—for example one encoding a sequence specific DNA-endonuclease—is expressed. An operably linkage does not necessarily require a direct linkage in the chemical sense. Genetic control sequences such as, for example, enhancer sequences are also capable of exerting their function on the target sequence from positions located at a distance or indeed other DNA molecules. Preferred arrangements are those in which the nucleic acid sequence to be expressed—for example one encoding a sequence specific DNA-endonuclease—is positioned after a sequence acting as promoter so that the two sequences are linked covalently to one another. The distance between the promoter sequence and the nucleic acid sequence—for example one encoding a sequence specific DNA-endonuclease—is preferably less than 200 base pairs, especially preferably less than 100 base pairs, very especially preferably less than 50 base pairs. The skilled worker is familiar with a variety of ways in order to obtain such an expression cassette. References for customary recombination and cloning techniques as given below. However, an expression cassette may also be constructed in such a way that the nucleic acid sequence to be expressed (for example one encoding a marker sequence, an agronomically valuable trait, or a sequence specific endonuclease) is brought under the control of an endogenous genetic control element, for example an endogenous promoter, for example by means of homologous recombination or else by random insertion. Such constructs are likewise understood as being expression cassettes for the purposes of the invention.

A “genetically-modified organism” or “GMO” refers to any organism that comprises transgene DNA. Exemplary organisms include plants, animals and microorganisms.

Homology between two nucleic acid sequences is understood as meaning the identity of the nucleic acid sequence over in each case the entire sequence length which is calculated by alignment with the aid of the program algorithm GAP (Wisconsin Package Version 10.0, University of Wisconsin, Genetics Computer Group (GCG), Madison, USA), setting the following parameters:
Gap Weight: 12 Length Weight: 4
Average Match: 2,912 Average Mismatch: −2,003

“Genome” or “genomic DNA” is conferring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

### DETAILED DESCRIPTION OF THE INVENTION

Accordingly, a first embodiment of the invention relates to a method for producing a transgenic plant comprising:
1) crossing a first transgenic plant comprising in its genome a DNA construct comprising
   a1) at least one recognition sequence of at least 10 base pairs for the site-directed induction of DNA double-strand breaks by a sequence specific DNA-endonuclease and
   b1) a nucleic acid sequence to be excised,
   wherein said elements a1) and b) and optionally further elements are flanked by homology sequences A and A′, having sufficient length and sufficient homology in order to ensure homologous recombination between A and A′, and having an orientation which upon recombination between A and A′—will lead to an excision of said elements a1) and b), and
   c1) at least one additional sequence conferring to said plant an agronomically valuable trait, wherein said sequence is not localized between the homology sequences A and A′ and would not be excised from the genome upon recombination between A and A′
   with a second transgenic plant comprising in its genome an expression cassette comprising
   a2) the parsley ubiquitin promoter, and operably linked thereto
   b2) a nucleic acid sequence coding for a sequence specific DNA-endonuclease having a sequence specificity for said recognition sequence a1),
2) generating descendants (F1) following this crossing, and—optionally—sexually or asexually generating further descendants, and
3) isolating descendants which have undergone recombination between the homology sequences A and A′ and which do not comprise in their genome said elements a1) and b1) but comprise sequence c1).

In an preferred embodiment the element b1) is an expression cassette for a marker sequence. More preferably, this expression cassette enables the expression of a sequence allowing selection of transformed plant material, wherein the DNA sequence encoding said selectable sequence is operably linked with a promoter functional in plants. However, excision may also be advantageous in other circumstance and for other non-marker sequences, for example, in cases of hybrid technology or trait containment.

In a preferred embodiment the orientation of the homology sequences in the DNA construct of the invention is in the form of directed repeats, which are flanking elements a1) and b1) and optionally further elements.

In another preferred embodiment the expression cassette for the endonuclease is segregated from the sequences c1) for the agronomically valuable trait by e.g., conventional breeding techniques. Plants are isolated which comprise sequence c1) but not said expression cassette for the endonuclease. In a preferred embodiment the resulting plant is marker free or selection marker free.

In another preferred embodiment the DNA construct of the invention, comprises two recognition sequences a1). It is especially preferred that these two recognition sequences are flanking the marker sequence (and optionally further elements) in a way that a cleavage at this two sides excises the marker sequence (and optionally further elements).

Other embodiments of the invention are related to vector comprising said DNA construct, and transgenic plants comprising said vector or said DNA construct.

The present invention enables sequences (such as marker sequences e.g., genes for resistance to antibiotics or herbicides) to be deleted from the genome (e.g., chromosomal DNA) of a plant organism in an accurately predictable manner with high efficiency.

Within the method of the invention it is an essential feature that two plants are crossed, each of these comprising a specific DNA construct:
i) a first plant (hereinafter the “endonuclease master plant”) comprising a expression cassette for expression of a sequence specific DNA-endonuclease (hereinafter the “endonuclease expression cassette”). Expression here is under control of the parsley ubiquitin promoter as specified in more detail below.
ii) a second plant (hereinafter the “trait plant”) comprising a recombination cassette for excision (hereinafter the “excision cassette”) of a sequence to be deleted (e.g., a marker sequences) and further comprising—optionally—sequences (e.g., an expression cassette) for an agronomically valuable trait.

The sequence to be eliminated (e.g., the marker sequence) is flanked by homology sequences A and A′ having sufficient length and sufficient homology in order to ensure homologous recombination between A and A′, and having an orientation which—upon recombination between A and A′—will lead to an excision of said sequence (e.g., the marker sequence) from the genome. Efficiency and accuracy of homologous recombination between A and A′ is mediated by action of a sequence specific DNA-endonuclease, which is able to cleave at a recognition site between the two homology sequences, inducing a double-strand break, and in consequence, triggering said homologous recombination between A and A′. By this homologous recombination also the recognition sequence for the sequence specific DNA-endonuclease is excised likewise, which allows the method of the invention to be used repeatedly for further controlled genetic modifications. The sequences which are deleted are those located between the homology sequences A and A′. In contrast to systems such as, for example, the cre/lox or the FRT/FLP system, one is not bound to specific sequences when performing recombination. The skilled worker knows that any sequence can undergo homologous recombination with another sequence provided that sufficient length and homology exist.

It is another inventive feature of the present invention, that expression of the sequence specific DNA-endonuclease is mediated by the parsley ubiquitin promoter. The method of the invention has at least four advantageous effects:
1) The use of parsley ubiquitin promoters for the endonuclease expression cassette surprisingly outmatches all other constitutive promoters tested so far including the gold-standard CaMV 35S promoter. The performance of the parsley ubiquitin promoter is by far better than the performance of any other promoter tested under equivalent conditions (see comparison examples). The parsley ubiquitin promoter seems to regulate efficient transcription and expression of the endonucleases in tissues and at times which are essential to allow induced homologous recombination. In consequence, many more cells of the F1 generation of a cross between an endonuclease master plant and a trait plant contain the respective recombination event. Therefore, the isolation of plants having the recombination event present in all cells is highly facilitated. Accordingly, the specific use of the parsley ubiquitin promoter solves problems which still adhere to constitutive promoters such as 35S CaMV and others.
2) Physical separation of the expression cassette for the endonuclease and the excision cassette (comprising its recognition sequences) by employing separate plants prevents premature excision that may occur in a co-transformation approach with both constructs and which may negatively affect the transformation/selection efficiency.
3) The method of the invention reduces multiple insertion (e.g., of a T-DNA) in one genomic location to a single insertion event by excision of the redundant copies (FIG. 10) in addition to the excision of the sequence to be eliminated (e.g., the selection marker).
4) The fact that the endonuclease is expressed from a construct in a separate plant allows for generation and use of a master plant. This means, that this master plant for the endonuclease can be crossed with various plants comprising different recombination cassettes (for the introduction of different agronomically valuable traits). This makes the method time and work efficient since only one of the two plants employed need to be generated for a new approach. Moreover, such endonuclease master plant could be in elite germplasm. Thus, upon crossing to the trait plant one could already initiate the first cross to breed the agronomical valuable trait into elite germplasm.

### 1. SEQUENCE SPECIFIC DNA ENDONUCLEASE

“Sequence specific DNA-endonuclease” generally refers to all those enzymes which are capable of generating double-strand breaks in double stranded DNA in a sequence-specific manner at one or more recognition sequences. Said DNA cleavage may result in blunt ends, or so called “sticky” ends of the DNA (having a 5′- or 3′-overhang). The cleavage site may be localized within or outside the recognition sequence. Various kinds of endonucleases can be employed. Endonucleases can be, for example, of the Class II or Class IIs type. Class IIs R-M restriction endonucleases catalyze the DNA cleavage at sequences other than the recognition sequence, i.e. they cleave at a DNA sequence at a particular number of nucleotides away from the recognition sequence (Szybalski et al. (1991) Gene 100:13-26). The following may be mentioned by way of example, but not by limitation:
1. Restriction endonucleases (e.g., type II or IIs), preferably homing endonucleases as described in detail herein below.
2. Chimeric or synthetic nucleases as described in detail herein below.

Unlike recombinases, restriction enzymes typically do not ligate DNA, but only cleave DNA. Restriction enzymes are described, for instance, in the New England Biolabs online catalog (www.neb.com), Promega online catalog (www.promega.com) and Rao et al. (2000) Prog Nucleic Acid Res Mol Biol 64:1-63. Within this invention “ligation” of the DNA ends resulting from the cleavage by the endonuclease is realized by fusion by homologous recombination of the homology sequences. The enzymes facilitating homologous recombination are naturally provided by the plant.

Preferably, the endonuclease is chosen in a way that its corresponding recognition sequences are rarely, if ever, found in the unmodified genome of the target plant organism. Ideally, the only copy (or copies) of the recognition sequence in the genome is (or are) the one(s) introduced by the DNA construct of the invention, thereby eliminating the chance that other DNA in the genome is excised or rearranged when the sequence-specific endonuclease is expressed.

One criterion for selecting a suitable endonuclease is the length of its corresponding recognition sequence. Said recognition sequence has an appropriate length to allow for rare cleavage, more preferably cleavage only at the recognition sequence(s) comprised in the DNA construct of the invention. One factor determining the minimum length of said recognition sequence is—from a statistical point of view—the size of the genome of the host organism. In an preferred embodiment the recognition sequence has a length of at least 10 base pairs, preferably at least 14 base pairs, more preferably at least 16 base pairs, especially preferably at least 18 base pairs, most preferably at least 20 base pairs.

A restriction enzyme that cleaves a 10 base pair recognition sequence is described in Huang B et al. (1996) J Protein Chem 15(5):481-9.

Suitable enzymes are not only natural enzymes, but also synthetic enzymes. Preferred enzymes are all those sequence specific DNA-endonucleases whose recognition sequence is known and which can either be obtained in the form of their proteins (for example by purification) or expressed using their nucleic acid sequence. This is why homing endonucleases are very especially preferred (Review: (Belfort M and Roberts R J (1997) Nucleic Acids Res 25: 3379-3388; Jasin M (1996) Trends Genet. 12:224-228; Internet: http://rebase.neb.com/rebase/rebase.homing.html). Owing to their long recognition sequences, they have no, or only a few, further recognition sequences in the chromosomal DNA of eukaryotic organisms in most cases.

The sequences encoding for such homing endonucleases can be isolated for example from the chloroplast genome of Chlamydomonas (Turmel M et al. (1993) J Mol Biol 232: 446-467). They are small (18 to 26 kD) and their open reading frames (ORF) have a “codon usage” which is suitable directly for nuclear expression in eukaryotes (Monnat R J Jr et al., (1999) Biochem Biophys Res Com 255:88-93). Homing endonucleases which are very especially preferably isolated are the homing endonucleases I-SceI (WO96/14408), I-SceII (Sarguiel B et al., (1990) Nucleic Acids Res 18:5659-5665), ISceIII (Sarguiel B et al. (1991) Mol Gen Genet. 255:340-341), I-CeuI (Marshall (1991) Gene 104:241-245), I-CreI (Wang J et al. (1997) Nucleic Acids Res 25: 3767-3776), I-ChuI (Cote V et al. (1993) Gene 129:69-76), I-TevI (Chu et al. (1990) Proc Natl Acad Sci USA 87:3574-3578; Bell-Pedersen et al. (1990) Nucleic Acids Res 18:3763-3770), I-TevII (Bell-Pedersen et al. (1990) Nucleic Acids Res 18:3763-3770), I-TevIII (Eddy et al. (1991) Genes Dev. 5:1032-1041), Endo SceI (Kawasaki et al. (1991) J Biol Chem 266:5342-5347), I-CpaI (Turmel M et al. (1995a) Nucleic Acids Res 23:2519-2525) and I-CpaII (Turmel M et al. (1995b) Mol. Biol. Evol. 12, 533-545).

Further homing endonucleases are detailed in the abovementioned Internet website, and examples which may be mentioned are homing endonucleases such as F-SceI, FSceII, F-SuvI, F-TevI, F-TevII, I-AmaI, I-AniI, I-CeuI, I-CeuAlIP, I-ChuI, I-CmoeI, I-CpaI, I-CpaII, I-CreI, I-CrepsbIP, I-CrepsbIIP, I-CrepsbiIIP, I-CrepsbIVP, I-CsmI, I-CvuI, ICvuAlP, I-DdiI, I-DdiII, I-DirI, I-DmoI, I-HmuI, I-HmuII, I-HspNIP, I-LiaI, I-MsoI, I-NaaI, I-NanI, I-Nc/IP, I-NgrIP, I-NitI, I-NjaI, I-Nsp236IP, I-PakI, I-PboIP, I-PcuIP, I-PcuAI, IPcuVI, I-PgrIP, I-PobIP, I-PorI, I-PorlIP, I-PpbIP, I-PpoI, I-SPBetaIP, I-ScaI, I-SceI, ISceII, I-SceII, I-SceIV, I-SceV, I-SceVI, I-SceVII, I-SexIP, I-SneIP, I-SpomCP, ISpomIP, I-SpomIIP, I-SquIP, I-Ssp6803I, I-SthPhiJP, I-SthPhiST3P, I-SthPhiS3P, ITdeIP, I-TevI, I-TevII, I-TevIII, I-UarAP, I-UarHGPA1P, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-PspI, PI-Rma438121P, PI-SPBetaIP, PI-SceI, PI-TfuI, PI-TfuII, PI-ThyI, PI-TliI, PI-TliII, H-DreI, I-BasI, I-BmoI, I-PogI, I-TwoI, PI-MgaI, PI-PabI, PI-PabI.

Preferred in this context are the homing endonucleases whose gene sequences are already known, such as, for example, F-SceI, I-CeuI, I-ChuI, I-DmoI, I-CpaI, I-CpaII, I-CreI, I-CsmI, F-TevI, F-TevII, I-TevII, I-TevII, I-AnII, I-CvuI, I-DdiI, I-HmuI, I-HmuII, I-LIaI, I-NanI, I-MsoI, I-NitI, I-NjaI, I-PakI, I-PorI, I-PpoI, I-ScaI, I-Ssp68031, PI-PkoI, PI-PkoII, PI-PspI, PI-TfuI, PI-TliI. Especially preferred are commercially available homing endonucleases such as I-CeuI, I-SceI, I-DmoI, I-PpoI, PI-PspI or PI-SceI. Endonucleases with particularly long recognition sequences, and which therefore only rarely (if ever) cleave within a genome include: I-CeuI (26 bp recognition sequence), PI-PspI (30 bp recognition sequence), PI-SceI (39 bp recognition sequence), I-SceI (18 bp recognition sequence) and I-PpoI (15 bp recognition sequence).

The enzymes can be isolated from their organisms of origin in the manner with which the skilled worker is familiar, and/or their coding nucleic acid sequence can be cloned. The sequences of various enzymes are deposited in GenBank.

Very especially preferred are the homing endonucleases I-SceI, I-CpaI, I-CpaII, I-CreI and I-ChuI. Sequences encoding said nucleases are known in the art and—for example—specified in WO 03/004659 (e.g., as SEQ ID NO: 2, 4, 6, 8, and 10 of WO 03/004659 hereby incorporated by reference).

In an preferred embodiment, the sequences encoding said homing endonucleases can be modified by insertion of an intron sequence. This prevents expression of a functional enzyme in procaryotic host organisms and thereby facilitates cloning and transformations procedures (e.g., based on E. coli or Agrobacterium). In plant organisms, expression of a functional enzyme is realized, since plants are able to recognize and “splice” out introns. Preferably, introns are inserted in the homing endonucleases mentioned as preferred above (e.g., into I-SceI or 1-CreI). Another preferred embodiment of the invention is related to a intron-comprising I-Sce-I sequence and its use in methods of the invention (more preferably a sequence as described by SEQ ID NO: 14).

In some aspects of the invention, molecular evolution can be employed to create an improved endonuclease. Polynucleotides encoding a candidate endonuclease enzyme can, for example, be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer (1994) Proc Natl Acad Sci USA 91:10747-10751; Stemmer (1994) Nature 370:389-391; and U.S. Pat. No. 5,605,793, U.S. Pat. No. 5,837,458, U.S. Pat. No. 5,830,721 and U.S. Pat. No. 5,811,238.

Other synthetic sequence specific DNA-endonucleases which may be mentioned by way of example are chimeric nucleases which are composed of an unspecific nuclease domain and a sequence-specific DNA binding domain consisting of zinc fingers (Bibikova M et al. (2001) Mol Cell Biol. 21:289-297). These DNA-binding zinc finger domains can be adapted to suit any DNA sequence. Suitable methods for preparing suitable zinc finger domains are described and known to the skilled worker (Beerli R R et al., Proc Natl Acad Sci USA. 2000; 97 (4):1495-1500; Beerli R R, et al., J Biol Chem 2000; 275(42):32617-32627; Segal D J and Barbas C F 3rd., Curr Opin Chem Biol 2000; 4(1):34-39; Kang J S and Kim J S, J Biol Chem 2000; 275(12):8742-8748; Beerli R R et al., Proc Natl Acad Sci USA 1998; 95(25):14628-14633; Kim J S et al., Proc Natl Acad Sci USA 1997; 94(8):3616-3620; Klug A, J Mol Biol 1999; 293(2):215-218; Tsai S Y et al., Adv Drug Deliv Rev 1998; 30(1-3):23-31; Mapp A K et al., Proc Natl Acad Sci USA 2000; 97(8):3930-3935; Sharrocks A D et al., Int J Biochem Cell Biol 1997; 29(12):1371-1387; Zhang L et al., J Biol Chem 2000; 275(43):33850-33860).

The sequence specific DNA-endonuclease may be expressed as a fusion protein with a nuclear localization sequence (NLS). This NLS sequence enables facilitated transport into the nucleus and increases the efficacy of the recombination system. A variety of NLS sequences are known in the art (Jicks G R and Raikhel N V (1995) Annu Rev Cell Biol 11:155-188; WO 03/004659). Preferred for plant organisms is, for example, the NLS sequence of the SV40 large antigen. However, owing to the small size of many sequence specific DNA-endonucleases (such as, for example, the homing endonucleases), a NLS sequence is not necessarily required. These enzymes are capable of passing through the nuclear pores even without an additional NLS.

In a further preferred embodiment, the activity of the sequence specific DNA-endonuclease can be induced. Suitable methods have been described for sequence-specific recombinases (Angrand P O et al. (1998) Nucl. Acids Res. 26(13):3263-3269; Logie C and Stewart A F (1995) Proc Natl Acad Sci USA 92(13):5940-5944; Imai T et al. (2001) Proc Natl Acad Sci USA 98(1):224-228). These methods employ fusion proteins of the sequence specific DNA-endonuclease and the ligand binding domain for steroid hormone receptor (for example the human androgen receptor, or mutated variants of the human estrogen receptor as described therein). Induction may be effected with ligands such as, for example, estradiol, dexamethasone, 4-hydroxytamoxifen or raloxifen.

Some sequence specific DNA-endonucleases enzymes are active as dimers (homo- or heterodimers; I-CreI forms a homodimer; I-SecIV forms a heterodimer) (Wernette C M (1998) Biochemical & Biophysical Research Communications 248(1):127-333)). Dimerization can be designed as an inducible feature, for example by exchanging the natural dimerization domains for the binding domain of a low-molecular-weight ligand. Addition of a dimeric ligand then brings about dimerization of the fusion protein. Corresponding inducible dimerization methods, and the preparation of the dimeric ligands, have been described (Amara J F et al. (1997) Proc Natl Acad Sci USA 94(20): 10618-1623; Muthuswamy S K et al. (1999) Mol Cell Biol 19(10):6845-685; Schultz L W and Clardy J (1998) Bioorg Med Chem. Lett. 8(1):1-6; Keenan T et al. (1998) Bioorg Med. Chem. 6(8): 1309-1335).

### 2. RECOGNITION SEQUENCES FOR SEQUENCE SPECIFIC DNA ENDONUCLEASE

“Recognition sequence” refers to a DNA sequence that is recognized by a sequence-specific DNA endonuclease of the invention. The recognition sequence will typically be at least 10 base pairs long, is more usually 10 to 30 base pairs long, and in most embodiments, is less than 50 base pairs long.

“Recognition sequence” generally refers to those sequences which, under the conditions in a plant cell used within this invention, enable the recognition and cleavage by the sequence specific DNA-endonuclease. The recognition sequences for the respective sequence specific DNA-endonucleases are mentioned in Table 1 hereinbelow by way of example, but not by limitation.

TABLE 1

Recognition sequences and organisms of origin of sequence specific
DNA-endonuclease (“\hat{ }” indicates the cleavage site of the sequence
specific DNA-endonuclease within recognition sequence).

| | Organisim | |
|---|---|---|
| Nuclease | of origin | Recognition sequence |
| I-AniI | Aspergillus | 5′-TTGAGGAGGTT\hat{ }TCTCTGTAAATAANNNNNNNNNNNNNNN |
| | nidulans | 3′-AACTCCTCCAAAGAGACATTTATTNNNNNNNNNNNNNNN\hat{ } |
| I-DdiI | Dictyostelium | 5′-TTTTTTGGTCATCCAGAAGTATAT |
| | discoideumAX3 | 3′-AAAAAACCAG\hat{ }TAGGTCTTCATATA |
| I-CvuI | Chlorella vulgaris | 5′-CTGGGTTCAAAACGTCGTGA\hat{ }GACAGTTTGG |
| | | 3′-GACCCAAGTTTTGCAG\hat{ }CACTCTGTCAAACC |
| I-CsmI | Chlamydomonas | 5′-GTACTAGCATGGGGTCAAATGTCTTTCTGG |
| | smithii | |
| I-CmoeI | Chlamydomonas- | 5′-TCGTAGCAGCT\hat{ }CACGGTT |
| | moewusii | 3′-AGCATCG\hat{ }TCGAGTGCCAA |
| I-CreI | Chlamydomonas- | 5′-CTGGGTTCAAAACGTCGTGA\hat{ }GACAGTTTGG |
| | reinhardtii | 3′-GACCCAAGTTTTGCAG\hat{ }CACTCTGTCAAACC |
| I-ChuI | Chlamydomonas | 5′-GAAGGTTTGGCACCTCG\hat{ }ATGTCGGCTCATC |
| | humicola | 3′-CTTCCAAACCGTG\hat{ }GAGCTACAGCCGAGTAG |
| I-CpaI | Chlamydomonas | 5′-CGATCCTAAGGTAGCGAA\hat{ }ATTCA |
| | pallidostigmatica | 3′-GCTAGGATTCCATC\hat{ }GCTTTAAGT |
| I-CpaII | Chlamydomonas | 5′-CCCGGCTAACTC\hat{ }TGTGCCAG |
| | pallidostigmatica | 3′-GGGCCGAT\hat{ }TGAGACACGGTC |
| I-CeuI | Chlamydomonas | 5′-CGTAACTATAACGGTCCTAA\hat{ }GGTAGCGAA |
| | eugametos | 3′-GCATTGATATTGCCAG\hat{ }GATTCCATCGCTT |
| I-DmoI | Desulfuro- | 5′-ATGCCTTGCCGGGTAA\hat{ }GTTCCGGCGCGCAT |
| | coccus mobilis | 3′-TACGGAACGGCC\hat{ }CATTCAAGGCCGCGCGTA |
| I-SceI | Saccharomyces | 5′-AGTTACGCTAGGGATAA\hat{ }CAGGGTAATATAG |
| | cerevisiae | 3′-TCAATGCGATCCC\hat{ }TATTGTCCCATTATATC |
| | | 5′-TAGGGATAA\hat{ }CAGGGTAAT |
| | | 3′-ATCCC\hat{ }TATTGTCCCATTA (“Core”-Sequence) |
| I-SceII | S. cervisiae | 5′-TTTTGATTCTTTGGTCACCC\hat{ }TGAAGTATA |
| | | 3′-AAAACTAAGAAACCAG\hat{ }TGGGACTTCATAT |
| I-SceIII | S. cervisiae | 5′-ATTGGAGGTTTTGGTAAC\hat{ }TATTTATTACC |
| | | 3′-TAACCTCCAAAACC\hat{ }ATTGATAAATAATGG |
| I-SceIV | S. cerevisiae | 5′-TCTTTTCTCTTGATTA\hat{ }GCCCTAATCTACG |
| | | 3′-AGAAAAGAGAAC\hat{ }TAATCGGGATTAGATGC |
| I-SceV | S. cerevisiae | 5′-AATAATTTTCT\hat{ }TCTTAGTAATGCC |
| | | 3′-TTATTAAAAGAAGAATCATTA\hat{ }CGG |
| I-SceVI | S. cerevisiae | 5′-GTTATTTAATG\hat{ }TTTTAGTAGTTGG |
| | | 3′-CAATAAATTACAAAATCATCA\hat{ }ACC |
| I-SceVII | S. cerevisiae | 5′-TGTCACATTGAGGTGCACTAGTTATTAC |
| PI-SceI | S. cerevisiae | 5′-ATCTATGTCGGGTGC\hat{ }GGAGAAAGAGGTAAT |
| | | 3′-TAGATACAGCC\hat{ }CACGCCTCTTTCTCCATTA |
| F-SceI | S. cerevisiae | 5′-GATGCTGTAGGC\hat{ }ATAGGCTTGGTT |
| | | 3′-CTACGACA\hat{ }TCCGTATCCGAACCAA |
| F-SceII | S. cerevisiae | 5′-CTTTCCGCAACA\hat{ }GTAAAATT |
| | | 3′-GAAAGGCG\hat{ }TTGTCATTTTAA |
| I-HmuI | Bacillus subtilis | 5′-AGTAATGAGCCTAACGCTCAGCAA |
| | bacteriophage | 3′-TCATTACTCGGATTGC\hat{ }GAGTCGTT |
| | SPO1 | |
| I-HmuII | Bacillus subtilis | 5′-AGTAATGAGCCTAACGCTCAACAANNNNNNNNNNNNNNNN- |
| | bacteriophage | NNNNNNNNNNNNNNNNNNNNNNN |
| | SP82 | |
| I-LlaI | Lactococcus lactis | 5′-CACATCCATAAC\hat{ }CATATCATTTTT |
| | | 3′-GTGTAGGTATTGGTATAGTAA\hat{ }AAA |
| I-MsoI | Monomastix | 5′-CTGGGTTCAAAACGTCGTGA\hat{ }GACAGTTTGG |
| | species | 3′-GACCCAAGTTTTGCAG\hat{ }CACTCTGTCAAACC |
| I-NanI | Naegleria | 5′-AAGTCTGGTGCCA\hat{ }GCACCCGC |
| | andersoni | 3′-TTCAGACC\hat{ }ACGGTCGTGGGCG |
| I-NitI | Naegleria italica | 5′-AAGTCTGGTGCCA\hat{ }GCACCCGC |
| | | 3′-TTCAGACC\hat{ }ACGGTCGTGGGCG |
| I-NjaI | Naegleria jamieso- | 5′-AAGTCTGGTGCCA\hat{ }GCACCCGC |
| | ni | 3′-TTCAGACC\hat{ }ACGGTCGTGGGCG |
| I-PakI | Pseudendoclonium | 5′-CTGGGTTCAAAACGTCGTGA\hat{ }GACCAGTTTGG |
| | akinetum | 3′-GACCCAAGTTTTGCAG\hat{ }CACTCTGTCAAACC |
| I-PorI | Pyrobaculum | 5′-GCGAGCCCGTAAGGGT\hat{ }GTGTACGGG |
| | organotrophum | 3′-CGCTCGGGCATT\hat{ }CCCACACATGCCC |
| I-PpoI | Physarum | 5′-TAACTATGACTCTCTTAA\hat{ }GGTAGCCAAAT |
| | polycephalum | 3′-ATTGATACTGAGAG\hat{ }AATTCCATCGGTTTA |
| I-ScaI | Saccharomyces | 5′-TGTCACATTGAGGTGCACT\hat{ }AGTTATTAC |
| | capensis | 3′-ACAGTGTAACTCCAC\hat{ }GTGATCAATAATG |
| I-Ssp6803I | Synechocystis | 5′-GTCGGGCT\hat{ }CATAACCCGAA |
| | species | 3′-CAGCCCGAGTA\hat{ }TTGGGCTT |
| PI-PfuI | Pyrococcus | 5′-GAAGATGGGAGGAGGG\hat{ }ACCGGACTCAACTT |
| | furiosus Vc1 | 3′-CTTCTACCCTCC\hat{ }TCCCTGGCCTGAGTTGAA |
| PI-PfuII | Pyrococcus | 5′-ACGAATCCATGTGGAGA\hat{ }AGAGCCTCTATA |
| | furiosus Vc1 | 3′-TGCTTAGGTACAC\hat{ }CTCTTCTCGGAGATAT |
| PI-PkoI | Pyrococcus koda- | 5′-GATTTTAGAT\hat{ }CCCTGTACC |
| | karaensis KOD1 | 3′-CTAAAA\hat{ }TCTAGGGACATGG |
| PI-PkoII | Pyrococcus koda- | 5′-CAGTACTACG\hat{ }GTTAC |
| | karaensis KOD1 | 3′-GTCATG\hat{ }ATGCCAATG |
| PI-PspI | Pyrococcus sp. | 5′-AAAATCCTGGCAAACAGCTATTAT\hat{ }GGGTAT |
| | | 3′-TTTTAGGACCGTTTGTCGAT\hat{ }AATACCCATA |
| PI-TfuI | Thermococcus | 5′-TAGATTTTAGGT\hat{ }CGCTATATCCTTCC |
| | fumicolans ST557 | 3′-ATCTAAAA\hat{ }TCCAGCGATATAGGAAGG |
| PI-TfuII | Thermococcus | 5′-TAYGCNGAYACN\hat{ }GACGGYTTYT |
| | fumicolans ST557 | 3′-ATRCGNCT\hat{ }RTGNCTGCCRAARA |
| PI-ThyI | Thermococcus | 5′-TAYGCNGAYACN\hat{ }GACGGYTTYT |
| | hydrothermalis | 3′-ATRCGNCT\hat{ }RTGNCTGCCRAARA |
| PI-TliI | Thermococcus | 5′-TAYGCNGAYACNGACGG\hat{ }YTTYT |
| | litoralis | 3′-ATRCGNCTRTGNC\hat{ }TGCCRAARA |
| PI-TliII | Thermococcus | 5′-AAATTGCTTGCAAACAGCTATTACGGCTAT |
| | litoralis | |
| I-TevI | Bacteriophage T4 | 5′-AGTGGTATCAAC\hat{ }GCTCAGTAGATG |
| | | 3′-TCACCATAGT\hat{ }TGCGAGTCATCTAC |
| I-TevII | Bacteriophage T4 | 5′-GCTTATGAGTATGAAGTGAACACGT\hat{ }TATTC |
| | | 3′-CGAATACTCATACTTCACTTGTG\hat{ }CAATAAG |
| F-TevI | Bacteriophage T4 | 5′-GAAACACAAGA\hat{ }AATGTTTAGTAAANNNNNNNNNNNNNN |
| | | 3′-CTTTGTGTTCTTTACAAATCATTTNNNNNNNNNNNNNN\hat{ } |
| F-TevII | Bacteriophage T4 | 5′-TTTAATCCTCGCTTC\hat{ }AGATATGGCAACTG |
| | | 3′-AAATTAGGAGCGA\hat{ }AGTCTATACCGTTGAC |
| H-DreI | E. coli pl-Drel | 5′-CAAAACGTCGTAA\hat{ }GTTCCGGCGCG |
| | | 3′-GTTTTGCAG\hat{ }CATTCAAGGCCGCGC |
| I-BasI | Bacillus | 5′-AGTAATGAGCCTAACGCTCAGACAA |
| | thuringiensis | 3′-TCATTACGAGTCGAACTCGGATTG |
| | phage Bastille | |
| I-BmoI | Bacillus mojaven- | 5′-GAGTAAGAGCCCG\hat{ }TAGTAATGACATGGC |
| | sis s87-18 | 3′-CTCATTCTCG\hat{ }GGCATCATTACTGTACCG |
| I-PogI | Pyrobaculum ogu- | 5′-CTTCAGTAT\hat{ }GCCCCGAAAC |
| | niense | 3′-GAAGT\hat{ }CATACGGGGCTTG |
| I-TwoI | Staphylococcus | 5′-TCTTGCACCTACACAATCCA |
| | aureus phage | 3′-AGAACGTGGATGTGTTAGGT |
| | Twort | |
| PI-MgaI | Mycobacterium | 5′-CGTAGCTGCCCAGTATGAGTCA |
| | gastri | 3′-GCATCGACGGGTCATACTCAGT |
| PI-PabI | Pyrococcus abyssi | 5′-GGGGGCAGCCAGTGGTCCCGTT |
| | | 3′-CCCCCGTCGGTCACCAGGGCAA |
| PI-PabII | Pyrococcus abyssi | 5′-ACCCCTGTGGAGAGGAGCCCCTC |
| | | 3′-TGGGGACACCTCTCCTCGGGGAG |

Also encompassed are minor deviations (degenerations) of the recognition sequence which still enable recognition and cleavage by the sequence specific DNA-endonuclease in question. Such deviations—also in connection with different framework conditions such as, for example, calcium or magnesium concentration—have been described (Argast G M et al. (1998) J Mol Biol 280: 345-353). Also encompassed are core sequences of these recognition sequences and minor deviations (degenerations) in there. It is known that the inner portions of the recognition sequences suffice for an induced double-strand break and that the outer ones are not absolutely relevant, but can codetermine the cleavage efficacy. Thus, for example, an 18 bp core sequence can be defined for I-SceI.

### 3. PROMOTERS OF THE INVENTION

Various promoters for expression in plants and plant cells can be employed in the invention. A first promoter—the parsley ubiquitin promoter—regulates the expression of the sequence-specific endonuclease. Other promoters may regulate the expression of the selection marker or the agronomically valuable trait.

### 3.1 Parsley Ubiquitin Promoter

Expression of the polynucleotide encoding a sequence-specific DNA endonuclease is controlled by a parsley ubiquitin promoter. The term “parsley ubiquitin promoter” mean the transcription regulating region of the ubiquitin gene from parsley (Petroselinum crispum), preferably the promoter sequences disclosed and claimed in international patent application WO 03/102198, hereby incorporated entirely by reference. More preferably, a parsley ubiquitin promoter is described by the nucleic acid sequence of SEQ ID NO: 8 or 15, and functional equivalents and functional equivalent fragments thereof.

Functional equivalents means transcription regulating sequences derived from a sequence as described by or obtainable from SEQ ID NO: 8 or 15 for example by substitution, insertion or deletion of one or more nucleotides which have a identity of at least 30%, preferably at least 50% or 70%, more preferably at least 90%, most preferably at least 95%, and demonstrate substantially the same transcription regulating properties than the parsley ubiquitin promoter as described by SEQ ID NO: 8 or 15. Functional equivalents may be obtained synthetically or from orthologous genes of other organisms by—for example—homology-based database screening or hybridization-based library screening.

Functionally equivalent fragments of a parsley ubiquitin promoter as described by SEQ ID NO: 8 or 15 can be obtained—for example—by deleting non-essential sequences without substantially modifying its transcription regulating properties. It is well known in the art that not all sequences in a promoter region are required for transcription regulation but that the essential regions are restricted to limited portions thereof (so called promoter elements). Functionally equivalent fragments of a promoter sequence can be obtained by deleting non-essential sequences (e.g., of a promoter sequence as described by SEQ ID NO: 8 or 15). Such a functionally equivalent fragment consists of at least 50, preferably at least 100, more preferably at least 150, most preferably at least 200 consecutive base pairs of a promoter as described by SEQ ID NO: 8 or 15 and has substantially the same promoter activity as the promoter described by SEQ ID NO: 8 or 15. Narrowing of a promoter sequence to specific, essential regulatory regions or elements can be facilitated by using computer algorithms for the prediction of promoter elements. In most promoters the essential regulatory regions are characterized by a clustering of promoter elements. A promoter element analysis can be done by computer programs like e.g., PLACE (“Plant Cis-acting Regulatory DNA Elements”; Higo K et al. (1999) Nucleic Acids Res 27:1, 297-300) or by using the B10BASE database “Transfac” (Biologische Datenbanken GmbH, Braunschweig).

A promoter activity of a functional equivalent or equivalent fragment is regarded substantially the same if transcription of a specific nucleic acid sequence under transcriptional control of such sequences does not derivate more than 50%, preferably more than 40%, more preferably more than 30% or 20%, most preferably more than 10% from a comparison value obtained under same conditions using the promoter sequence as described by SEQ ID NO: 8 or 15. The level of expression may be higher or lower than the standard value. Preferably the transcription level is assessed by expression of nucleic acids encoding for readily quantifiable proteins such as reporter proteins (e.g., green fluorescence protein (GFP); Chui et al. (1996) Curr Biol 6: 325-330; Leffel S M et al. (1997) Biotechniques. 23(5):912-8), chloramphenicol transferase, luciferase (Millar et al. (1992) Plant Mol Biol Rep 10:324-414), β-galactosidase, or—preferably—β-glucuronidase (Jefferson et al. (1987) EMBO J. 6:3901-3907).

A functional equivalent preferably comprises one or more of the promoter elements identified in the parsley ubiquitin promoter presumably constituting its essential parts for transcription regulation, such elements may be identified by computer algorithms such as PLACE (Higo et al. (1999) Nucl. Acid. Res., Vol. 27, No. 1, 297-300) and may be selected from the group consisting of:
a) a putative heat shock inducible element (=HSE) at a position equivalent to position base 534-547 of SEQ ID NO: 8,
b) two CAAACAC-elements at a position equivalent to position base 264 to 270, and 716 (complementary strand) of SEQ ID NO: 8 (Stalberg K et al. (1996) Planta 199:515-519)
c) two AACAAAC-elements at a position equivalent to position base 140 to 146, and 461 (complementary strand) (Wu C et al. (2000) Plant J 23: 415-421)
d) a TATA-Box (TATATATA) at a position equivalent to position base 291 to 297 of SEQ ID NO: 8 and close to the expected transcription start at position 237 (Joshi C P (1987) Nucleic Acids Res 15(16):6643-53)
e) all together 4 ACGTA-boxes (at a position equivalent to position base 214, 674, 692, and 880, respectively)
f) abscisic acid responsive element (at a position equivalent to position base 227 of SEQ ID NO: 8) (Hattori T et al. (2002) Plant Cell Physiol 43: 136-140);
g) several amylase-boxes at a position equivalent to position base 139, 421 (complementary strand), 462 (complementary strand), 789 (complementary strand), and 871 (complementary strand), respectively, of SEQ ID NO: 8 (Huang N et al. (1990) Plant Mol Biol 14:655-668)
h) a CACGTG motif at a position equivalent to position base 565 of SEQ ID NO: 8 (Menkens A E (1995) Trends in Biochemistry 20:506-510)
i) altogether 16 GATA boxes (Gilmartin P M et al. (1990) Plant Cell 2:369-378)
j) 5 GT1 consensus binding sites (GRWAAW) at a position equivalent to position base 395, and on the complementary strand at position 52, 387, 504, and 647, respectively, of SEQ ID NO: 8 (Villain P et al (1996) J Biol Chem 271:32593-32598)
k) a Ibox (GATAAG) at a position equivalent to position base 474 of SEQ ID NO: 8 (complementary strand) (Rose A et al. (1999) Plant J 20:641-652)
l) a LTRE (low-temperature-responsive element) CCGAAA at a position equivalent to position base 632 of SEQ ID NO: 8 (Dunn M A et al. (1998) Plant Mol Biol 38:551-564)
m) several binding sites for various classes of myb transcription factors (Jin H et al. (1999) Plant Mol Biol 41(5):577-85)
n) several W-box binding sites at a position equivalent to position base 549, 61, 550, and 919, respectively, of SEQ ID NO: 8, which are bound by WRKY transcription factors (Eulgem T et al. (2000) Trends Plant Sci 5:199-206).

Preferably the equivalent promoter comprises at least 1, 2, 3, 4, 5, 6, 7, 8, 9, 10, 11, 12, 13 or all of the above mentioned elements. Preferably the promoter comprises at least the elements a, b, c, and d.

Sequence comparison between the parsley ubiquitin promoter (PcUbi4-2) and the maize ubiquitin promoter demonstrates a very low identify of only 26% which is non-significant (Gap opening penalty 15, Gap extension penalty 6.66) (Altschul et al. (1990) Mol Biol 215:403-410, Altschul et al. (1997) Nucl. Acid Res 25:3389-3402). A BLAST search with one of sequences in the GenBank database would not identify the other promoter. The homology between the coding regions for the ubiquitins from maize and P. crispum is on nucleic acid level as high as 66.1%.

Accordingly, another subject matter of the invention relates to a transgenic expression cassettes comprising a sequence coding for a homing endonuclease operably linked to a parsley ubiquitin promoter as defined above.

Other embodiments of the invention are related to a transgenic vector comprising said expression cassette, and transgenic plants or plant cells comprising in their genome, preferably in their nuclear, chromosomal DNA, said expression cassette or said vector. Enclosed are also cells, cell cultures, tissues, parts or propagation material—such as, for example, in the case of plant organisms leaves, roots, seeds, fruit, pollen and the like—derived from said transgenic plants.

Obviously, also the promoter controlling expression of the agronomically valuable trait or marker sequence may be a parsley ubiquitin promoter.

### 3.2 Promoter for General Use

Promoters for the expression of the marker sequence or the agronomically valuable trait can be selected from all promoter having activity in plants or parts thereof. These promoters are selected for the tissues or cells where expression of the marker sequence and/or trait gene is desired. A number of exemplary promoters are described below. The following promoters, however, are only provided as examples and are not intended to limit the invention. Those of skill in the art will recognize that other promoters with desired expression patterns are well known or can be selected with routine molecular techniques.

A promoter can be derived from a gene that is under investigation, or can be a heterologous promoter that is obtained from a different gene, or from a different species. Suitable promoters can be derived from plants or plant pathogens like e.g., plant ylruses. Where expression of a gene in all tissues of a transgenic plant or other organism is desired, one can use a “constitutive” promoter, which is generally active under most environmental conditions and states of development or cell differentiation (Benfey et al. (1989) EMBO J. 8:2195-2202). The promoter controlling expression of the trait gene and/or marker sequence can be constitutive. Suitable constitutive promoters for use in plants include, for example, the cauliflower mosaic virus (CaMV) 35S transcription initiation region (Franck et al. (1980) Cell 21:285-294; Odell et al. (1985) Nature 313:810-812; Shewmaker et al. (1985) Virology 140:281-288; Gardner et al. 1986, Plant Mol. Biol. 6, 221-228), the 19S transcription initiation region (U.S. Pat. No. 5,352,605 and WO 84/02913), and region VI promoters, the 1′- or 2′-promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis (1997) Plant J 11(5): 1017-1027), the mas, Mac or DoubleMac promoters (U.S. Pat. No. 5,106,739; Comai et al. (1990) Plant Mol Biol 15:373-381), the ubiquitin promoter (Holtorf S et al. (1995) Plant Mol Biol 29:637-649) and other transcription initiation regions from various plant genes known to those of skill in the art. Useful promoters for plants also include those obtained from Ti- or Ri-plasmids, from plant cells, plant viruses or other organisms whose promoters are found to be functional in plants. Bacterial promoters that function in plants, and thus are suitable for use in the methods of the invention include the octopine synthetase promoter, the nopaline synthase promoter, and the mannopine synthetase promoter. Suitable endogenous plant promoters include the ribulose-1,6-biphosphate (RUBP) carboxylase small subunit (ssu) promoter, the α-conglycinin promoter, the phaseolin promoter, the ADH promoter, and heatshock promoters.

Of course, promoters can regulate expression all of the time in only one or some tissues. Alternatively, a promoter can regulate expression in all tissues but only at a specific developmental time point.

One can use a promoter that directs expression of a gene of interest in a specific tissue or is otherwise under more precise environmental or developmental control. Examples of environmental conditions that may affect transcription by inducible promoters include pathogen attack, anaerobic conditions, ethylene or the presence of light. Promoters under developmental control include promoters that initiate transcription only in certain tissues or organs, such as leaves, roots, fruit, seeds, or flowers, or parts thereof. The operation of a promoter may also vary depending on its location in the genome. Thus, an inducible promoter may become fully or partially constitutive in certain locations.

Examples of tissue-specific plant promoters under developmental control include promoters that initiate transcription only in certain organs or tissues, such as fruits, seeds, flowers, anthers, ovaries, pollen, the meristem, flowers, leaves, stems, roots and seeds. The tissue-specific ES promoter from tomato is particularly useful for directing gene expression so that a desired gene product is located in fruits. See, e.g., Lincoln et al. (1988) Proc Natl Acad Sci USA 84:2793-2797; Deikman et al. (1988) EMBO J. 7:3315-3320; Deikman et al. (1992) Plant Physiol 100:2013-2017. Other suitable seed specific promoters include those derived from the following genes: MAC1 from maize (Sheridan et al. (1996) Genetics 142:1009-1020, Cat3 from maize (GenBank No. L05934, Abler et al. (1993) Plant Mol Biol 22:10131-1038, the gene encoding oleosin 18 kD from maize (GenBank No. J05212, Lee et al. (1994) Plant Mol Biol 26:1981-1987), viviparous-1 from Arabidopsis (Genbank No. U93215), the gene encoding oleosin from Arabidopsis (Genbank No. Z17657), Atmycl from Arabidopsis (Urao et al. (1996) Plant Mol Biol 32:571-576, the 2s seed storage protein gene family from Arabidopsis (Conceicao et al. (1994) Plant 5:493-505) the gene encoding oleosin 20 kD from Brassica napus (GenBank No. M63985), napin from Brassica napus (GenBank No. J02798, Josefsson et al. (1987) J. Biol. Chem. 262:12196-12201), the napin gene family (e.g., from Brassica napus; Sjodahl et al. (1995) Planta 197:264-271, U.S. Pat. No. 5,608,152; Stalberg K, et al. (1996) L. Planta 199: 515-519), the gene encoding the 2S storage protein from Brassica napus (Dasgupta et al. (1993) Gene 133: 301-302), the genes encoding oleosin A (Genbank No. U09118) and oleosin B (Genbank No. U09119) from soybean, the gene encoding low molecular weight sulphur rich protein from soybean (Choi et al. (1995) Mol Gen Genet. 246:266-268), the phaseolin gene (U.S. Pat. No. 5,504,200, Bustos M M et al., Plant Cell. 1989; 1(9):839-53), the 2S albumin gene (Joseffson L G et al., (1987) J Biol Chem 262: 12196-12201), the legumin gene (Shirsat A et al. (1989) Mol Gen Genet. 215(2):326-331), the USP (unknown seed protein) gene (Bäumlein H et al. (1991) Mol Gen Genetics 225(3):459-67), the sucrose binding protein gene (WO 00/26388), the legumin B4 gene (LeB4; Bäumlein H et al. (1991) Mol Gen Genet. 225:121-128; Baeumlein et al. (1992) Plant J 2(2):233-239; Fiedler U et al. (1995) Biotechnology (NY) 13(10):1090-1093), the Ins Arabidopsis oleosin gene (WO9845461), the Brassica Bce4 gene (WO 91/13980), genes encoding the “high-molecular-weight glutenin” (HMWG), gliadin, branching enzyme, ADP-glucose pyrophosphatase (AGPase) or starch synthase. Furthermore preferred promoters are those which enable seed-specific expression in monocots such as maize, barley, wheat, rye, rice and the like. Promoters which may advantageously be employed are the promoter of the Ipt2 or Ipt1 gene (WO 95/15389, WO 95/23230) or the promoters described in WO 99/16890 (promoters of the hordein gene, the glutelin gene, the oryzin gene, the prolamine gene, the gliadin gene, the zein gene, the kasirin gene or the secalin gene).

Further suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP-A 409 625).

Promoters which are furthermore suitable are those which ensure leaf-specific expression. Promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus et al. (1989) EMBO J. 8(9):2445-2451). Other preferred promoters are those which govern expression in seeds and plant embryos.

Further suitable promoters are, for example, fruit-maturation-specific promoters such as, for example, the tomato fruit-maturation-specific promoter (WO 94/21794), flower-specific promoters such as, for example, the phytoene synthase promoter (WO 92/16635) or the promoter of the P-rr gene (WO 98/22593) or another node-specific promoter as described in EP-A 249676 may be used advantageously. The promoter may also be a pith-specific promoter, such as the promoter isolated from a plant TrpA gene as described in WO 93/07278.

A development-regulated promoter is, inter alia, described by Baerson et al. (Baerson S R, Lamppa G K (1993) Plant Mol Biol 22(2):255-67).

Other preferred promoters are promoters induced by biotic or abiotic stress, such as, for example, the pathogen-inducible promoter of the PRP1 gene (Ward et al., Plant Mol Biol 1993, 22: 361-366), the tomato heat-inducible hsp80 promoter (U.S. Pat. No. 5,187,267), the potato chill-inducible alpha-amylase promoter (WO 96/12814) or the wound-induced pinII promoter (EP375091).

Promoters may also encompass further promoters, promoter elements or minimal promoters capable of modifying the expression-specific characteristics. Thus, for example, the tissue-specific expression may take place in addition as a function of certain stress factors, owing to genetic control sequences. Such elements are, for example, described for water stress, abscisic acid (Lam E and Chua N H (1991) J Biol Chem 266(26):17131-17135) and heat stress (Schoffl F et al. (1989) Molecular & General Genetics 217(2-3):246-53).

### 4. THE HOMOLOGY SEQUENCES

Referring to the homology sequences (e.g., A, A′) “sufficient length” preferably refers to sequences with a length of at least 20 base pairs, preferably at least 50 base pairs, especially preferably at least 100 base pairs, very especially preferably at least 300 base pairs, most preferably at least 500 base pairs.

Referring to the homology sequences (e.g., A, A′), “sufficient homology” preferably refers to sequences with at least 70%, preferably 80%, by preference at least 90%, especially preferably at least 95%, very especially preferably at least 99%, most preferably 100%, homology within these homology sequences over a length of at least 20 base pairs, preferably at least 50 base pairs, especially preferably at least 100 base pairs, very especially preferably at least 300 base pairs, most preferably at least 500 base pairs.

The homology sequences A and A′ are preferably organized in the form of a direct repeat. The term “direct repeat” means a subsequent localization of two sequences on the same strand of a DNA molecule in the same orientation, wherein these two sequences fulfill the above given requirements for homologous recombination between said two sequences.

In an preferred embodiment, the homology sequences may be a duplication of a sequence having additional use within the DNA construct. For example, the homology sequences may be two transcription terminator sequences. One of these terminator sequences may be operably linked to the agronomically valuable trait, while the other may be linked to the marker sequence, which is localized in 3′-direction of the trait gene. Recombination between the two terminator sequences will excise the marker sequence but will reconstitute the terminator of the trait gene (see FIG. 4).

In another example, the homology sequences may be two promoter sequences. One of these promoter sequences may be operably linked to the agronomically valuable trait, while the other may be linked to the marker sequence, which is localized in 5′-direction of the trait gene. Recombination between the two promoter sequences will excise the marker sequence but will reconstitute the promoter of the trait gene (see FIG. 3).

The person skilled in the art will know that the homology sequences do not need to be restricted to a single functional element (e.g. promoter or terminator), but may comprise or extent to other sequences (e.g. being part of the coding region of the trait gene and the respective terminator sequence of said trait gene (see FIG. 5).

### 5. ADDITIONAL ELEMENTS IN THE DNA CONSTRUCT

The DNA construct may—beside the various promoter sequences—comprise additional genetic control sequences. The term “genetic control sequences” is to be understood in the broad sense and refers to all those sequences which affect the making or function of the DNA construct to the invention or an expression cassette comprised therein. Preferably, a expression cassettes according to the invention encompass 5′-upstream of the respective nucleic acid sequence to be expressed a promoter and 3′-downstream a terminator sequence as additional genetic control sequence, and, if appropriate, further customary regulatory elements, in each case in operable linkage with the nucleic acid sequence to be expressed.

Genetic control sequences are described, for example, in “Goeddel; Gene Expression Technology: Methods in Enzymology 185, Academic Press, San Diego, Calif. (1990)” or “Gruber and Crosby, in: Methods in Plant Molecular Biology and Biotechnolgy, CRC Press, Boca Raton, Fla., eds.: Glick and Thompson, Chapter 7, 89-108” and the references cited therein.

Examples of such control sequences are sequences to which inductors or repressors bind and thus regulate the expression of the nucleic acid. Genetic control sequences furthermore also encompass the 5′-untranslated region, introns or the noncoding 3′-region of genes. It has been demonstrated that they may play a significant role in the regulation of gene expression. Thus, it has been demonstrated that 5′-untranslated sequences are capable of enhancing the transient expression of heterologous genes. Furthermore, they may promote tissue specificity (Rouster J et al. (1998) Plant J 15:435-440.). Conversely, the 5′-untranslated region of the opaque-2 gene suppresses expression. Deletion of the region in question leads to an increased gene activity (Lohmer S et al. (1993) Plant Cell 5:65-73). Genetic control sequences may also encompass ribosome binding sequences for initiating translation. This is preferred in particular when the nucleic acid sequence to be expressed does not provide suitable sequences or when they are not compatible with the expression system.

The expression cassette can advantageously comprise one or more of what are known as enhancer sequences in operable linkage with the promoter, which enable the increased transgenic expression of the nucleic acid sequence. Additional advantageous sequences, such as further regulatory elements or terminators, may also be inserted at the 3′ end of the nucleic acid sequences to be expressed recombinantly. One or more copies of the nucleic acid sequences to be expressed recombinantly may be present in the gene construct. Genetic control sequences are furthermore understood as meaning sequences which encode fusion proteins consisting of a signal peptide sequence.

Polyadenylation signals which are suitable as genetic control sequences are plant polyadenylation signals, preferably those which correspond essentially to T-DNA polyadenylation signals from Agrobacterium tumefaciens. Examples of particularly suitable terminator sequences are the OCS (octopine synthase) terminator and the NOS (nopaline synthase) terminator. Also preferably are those taken from viral sequences, e.g. the 35S terminator.

The DNA constructs according to the invention and any vectors derived therefrom may comprise further functional elements. The term “further functional elements” is to be understood in the broad sense. It preferably refers to all those elements which affect the generation, multiplication, function, use or value of said DNA construct or vectors comprising said DNA construct, or cells or organisms comprising the before mentioned. These further functional elements may include but shall not be limited to:
i) Origins of replication which ensure replication of the expression cassettes or vectors according to the invention in, for example, E. coli. Examples which may be mentioned are OR1 (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).
ii) Multiple cloning sites (MCS) to enable and facilitate the insertion of one or more nucleic acid sequences.
iii) Sequences which make possible homologous recombination or insertion into the genome of a host organism.
iv) Elements, for example border sequences, which make possible the Agrobacterium-mediated transfer in plant cells for the transfer and integration into the plant genome, such as, for example, the right or left border of the T-DNA or the vir region.

### 6. THE MARKER SEQUENCE

The term “marker sequence” is to be understood in the broad sense to include all nucleotide sequences (and/or polypeptide sequences translated therefrom) which facilitate detection, identification, or selection of transformed cells, tissues or organism (e.g., plants). The terms “sequence allowing selection of a transformed plant material”, “selection marker” or “selection marker gene” or “selection marker protein” or “marker” have essentially the same meaning.

Markers may include (but are not limited to) selectable marker and screenable marker. A selectable marker confers to the cell or organism a phenotype resulting in a growth or viability difference. The selectable marker may interact with a selection agent (such as a herbicide or antibiotic or pro-drug) to bring about this phenotype. A screenable marker confers to the cell or organism a readily detectable phenotype, preferably a visibly detectable phenotype such a color or staining. The screenable marker may interact with a screening agent (such as a dye) to bring about this phenotype.

Selectable marker (or selectable marker sequences) comprise but are not limited to
a) negative selection marker, which confer a resistance against toxic (in case of plants phytotoxic) agent such as an antibiotic, herbicides or other biocides,
b) counter selection marker, which confer a sensitivity against certain chemical compounds (e.g., by converting a non-toxic compound into a toxic compound), and
c) positive selection marker, which confer a growth advantage (e.g., by expression of key elements of the cytokinin or hormone biosynthesis leading to the production of a plant hormone e.g., auxins, gibberllins, cytokinins, abscisic acid and ethylene; Ebinuma H et al. (2000) Proc Natl Acad Sci USA 94:2117-2121).

When using negative selection markers, only plants are selected which comprise said negative selection marker. When using counter selection marker, only plants are selected which lack said counter-selection marker. Counter-selection marker may be employed to verify successful excision of a sequence (comprising said counter-selection marker) from a genome. Screenable marker sequences include but are not limited to reporter genes (e.g. luciferase, glucuronidase, chloramphenicol acetyl transferase (CAT, etc.). Preferred marker sequences include but shall not be limited to:

i) Negative Selection Marker

As a rule, negative selection markers are useful for selecting cells which have successfully undergone transformation. The negative selection marker, which has been introduced with the DNA construct of the invention, may confer resistance to a biocide or phytotoxic agent (for example a herbicide such as phosphinothricin, glyphosate or bromoxynil), a metabolism inhibitor such as 2-deoxyglucose-6-phosphate (WO 98/45456) or an antibiotic such as, for example, tetracyclin, ampicillin, kanamycin, G 418, neomycin, bleomycin or hygromycin to the cells which have successfully undergone transformation. The negative selection marker permits the selection of the trans-formed cells from untransformed cells (McCormick et al. (1986) Plant Cell Reports 5:81-84). Negative selection marker in a vector of the invention may be employed to confer resistance in more than one organism. For example a vector of the invention may comprise a selection marker for amplification in bacteria (such as E. coli or Agrobacterium) and plants. Examples of selectable markers for E. coli include: genes specifying resistance to antibiotics, i.e., ampicillin, tetracycline, kanamycin, erythromycin, or genes conferring other types of selectable enzymatic activities such as galactosidase, or the lactose operon. Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc Natl Acad Sci USA 78:2072; Southern & Berg (1982) J Mol Appl Genet. 1: 327). Selection markers for plant cells often confer resistance to a biocide or an antibiotic, such as, for example, kanamycin, G 418, bleomycin, hygromycin, or chloramphenicol, or herbicide resistance, such as resistance to chlorsulfuron or Basta.

Especially preferred negative selection markers are those which confer resistance to herbicides. Examples of negative selection markers are:
DNA sequences which encode phosphinothricin acetyltransferases (PAT), which acetylates the free amino group of the glutamine synthase inhibitor phosphinothricin (PPT) and thus brings about detoxification of PPT (de Block et al. (1987) EMBO J. 6:2513-2518) (also referred to as Bialophos® resistance gene bar; EP 242236),
5-enolpyruvylshikimate-3-phosphate synthase genes (EPSP synthase genes), which confer resistance to Glyphosate® (N-(phosphonomethyl)glycine),
the gox gene, which encodes the Glyphosate®-degrading enzyme Glyphosate oxidoreductase,
the deh gene (encoding a dehalogenase which inactivates Dalapon®),
acetolactate synthases which confer resistance to sulfonylurea and imidazolinone,
bxn genes which encode Bromoxynil®-degrading nitrilase enzymes,
the kanamycin, or G418, resistance gene (NPTII). The NPTII gene encodes a neomycin phosphotransferase which reduces the inhibitory effect of kanamycin, neomycin, G418 and paromomycin owing to a phosphorylation reaction (Beck et al (1982) Gene 19: 327),
the DOG^{R}1 gene. The DOG^{R}1 gene has been isolated from the yeast Saccharomyces cerevisiae (EP 0 807 836). It encodes a 2-deoxyglucose-6-phosphate phosphatase which confers resistance to 2-DOG (Randez-Gil et al. (1995) Yeast 11:1233-1240).
the hyg gene, which codes for the enzyme hygromycin phosphotransferase and confers resistance to the antibiotic hygromycin (Gritz and Davies (1983) Gene 25: 179);
especially preferred are negative selection markers that confer resistance against the toxic effects imposed by D-amino acids like e.g., D-alanine and D-serine (WO 03/060133; Erikson 2004). Especially preferred as negative selection marker in this contest are the daol gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) (EC: 4.3. 1.18; GenBank Acc.-No.: J01603).
   ii) Positive Selection Marker

Positive selection marker comprise but are not limited to growth stimulating selection marker.asGenes like isopentenyltransferase from Agrobacterium tumefaciens (strain:PO22; Genbank Acc.-No.: AB025109) may—as a key enzyme of the cytokinin biosynthesis—facilitate regeneration of transformed plants (e.g., by selection on cytokinin-free medium). Corresponding selection methods are described (Ebinuma H et al. (2000) Proc Natl Acad Sci USA 94:2117-2121; Ebinuma H et al. (2000) Selection of Marker-free transgenic plants using the oncogenes (ipt, rol A, B, C) of Agrobacterium as selectable markers, In Molecular Biology of Woody Plants. Kluwer Academic Publishers). Additional positive selection markers, which confer a growth advantage to a transformed plant in comparison with a non-transformed one, are described e.g., in EP-A 0 601 092. Growth stimulation selection markers may include (but shall not be limited to) β-Glucuronidase (in combination with e.g., a cytokinin glucuronide), mannose-6-phosphate isomerase (in combination with mannose), UDP-galactose-4-epimerase (in combination with e.g., galactose), wherein mannose-6-phosphate isomerase in combination with mannose is especially preferred.

iii) Counter Selection Markers

Counter-selection markerenable the selection of organisms with successfully deleted sequences (Koprek T et al. (1999) Plant J 19(6):719-726). TK thymidine kinase (TK) and diphtheria toxin A fragment (DT-A), codA gene encoding a cytosine deaminase (Gleve A P et al. (1999) Plant Mol Biol 40(2):223-35; Pereat R1 et al. (1993) Plant Mol Biol 23(4):793-799; Stougaard J (1993) Plant J 3:755-761), the cytochrome P450 gene (Koprek et al. (1999) Plant J 16:719-726), genes encoding a haloalkane dehalogenase (Naested H (1999) Plant J 18:571-576), the iaaH gene (Sundaresan V et al. (1995) Genes & Development 9:1797-1810), the tms2 gene (Fedoroff N V & Smith D L (1993) Plant J 3:273-289), and D-amino acid oxidases causing toxic effects by conversion of D-amino acids (WO 03/060133).

In a preferred embodiment the excision cassette includes at least one of said counter-selection markers to distinguish plant cells or plants with successfully excised sequences from plant which still contain these. In a more preferred embodiment the excision cassette of the invention comprises a dual-function marker i.e. a marker with can be employed as both a negative and a counter selection marker depending on the substrate employed in the selection scheme. An example for a dual-function marker is the daol gene (EC: 1.4. 3.3: GenBank Acc.-No.: U60066) from the yeast Rhodotorula gracilis, which can be employed as negative selection marker with D.-amino acids such as D-alanine and D-serine, and as counter-selection marker with D-amino acids such as D-isoleucine and D-valine (see European Patent Appl. No.: 04006358.8)

iv) Screenable Marker (Reporter Genes)

Screenable marker (such as reporter genes) encode readily quantifiable or detectable proteins and which, via intrinsic color or enzyme activity, ensure the assessment of the transformation efficacy or of the location or timing of expression. Especially preferred are genes encoding reporter proteins (see also Schenborn E, Groskreutz D. (1999) Mol Biotechnol 13(1):29-44) such as
“green fluorescence protein” (GFP) (Chui W L et al. (1996) Curr Biol 6:325-330; Leffel S M et al., (1997) Biotechniques 23(5):912-8; Sheen et al. (1995) Plant J 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228).
Chloramphenicol transferase,
luciferase (Millar et al. (1992) Plant Mol Biol Rep 10:324-414; Ow et al. (1986) Science 234:856-859) permits selection by detection of bioluminescence,
β-galactosidase, encodes an enzyme for which a variety of chromogenic substrates are available,
β-glucuronidase (GUS) (Jefferson et al. (1987) EMBO J. 6:3901-3907) or the uidA gene, which encodes an enzyme for a variety of chromogenic substrates,
R locus gene product: protein which regulates the production of anthocyanin pigments (red coloration) in plant tissue and thus makes possible the direct analysis of the promoter activity without the addition of additional adjuvants or chromogenic substrates (Dellaporta et al. (1988) In: Chromosome Structure and Function: Impact of New Concepts, 18th Stadler Genetics Symposium, 11:263-282,),
β-lactamase (Sutcliffe (1978) Proc Natl Acad Sci USA 75:3737-3741), enzyme for a variety of chromogenic substrates (for example PADAC, a chromogenic cephalosporin),
xyIE gene product (Zukowsky et al. (1983) Proc Natl Acad Sci USA 80:1101-1105), catechol dioxygenase capable of converting chromogenic catechols,
α-amylase (Ikuta et al. (1990) Bio/technol. 8:241-242),
tyrosinase (Katz et al. (1983) J Gene Microbiol 129:2703-2714), enzyme which oxidizes tyrosine to give DOPA and dopaquinone which subsequently form melanine, which is readily detectable,
aequorin (Prasher et al. (1985) Biochem Biophys Res Commun 126(3):1259-1268), can be used in the calcium-sensitive bioluminescence detection.

### 7. TARGET ORGANISMS

The methods of the invention are useful for obtaining marker-free plants, or cells, parts, tissues, harvested material derived therefrom.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

Preferred are plants and plant materials of the following plant families: Amaranthaceae, Brassicaceae, Carophyllaceae, Chenopodiaceae, Compositae, Cucurbitaceae, Labiatae, Leguminosae, Papilionoideae, Liliaceae, Linaceae, Malvaceae, Rosaceae, Saxifragaceae, Scrophulariaceae, Solanaceae, Tetragoniaceae.

Annual, perennial, monocotyledonous and dicotyledonous plants are preferred host organisms for the generation of transgenic plants. The use of the recombination system, or method according to the invention is furthermore advantageous in all ornamental plants, useful or ornamental trees, flowers, cut flowers, shrubs or turf. Said plant may include—but shall not be limited to—bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae.

Plants for the purposes of the invention may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geraniums, Liliaceae such as drachaena, Moraceae such as ficus, Araceae such as philodendron and many others.

The transgenic plants according to the invention are furthermore selected in particular from among dicotyledonous crop plants such as, for example, from the families of the Leguminosae such as pea, alfalfa and soybean; Solanaceae such as tobacco and many others; the family of the Umbelliferae, particularly the genus Daucus (very particularly the species carota (carrot)) and Apium (very particularly the species graveolens dulce (celery)) and many others; the family of the Solanaceae, particularly the genus Lycopersicon, very particularly the species esculentum (tomato) and the genus Solanum, very particularly the species tuberosum (potato) and melongena (aubergine) and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others.

The transgenic plants according to the invention are selected in particular among monocotyledonous crop plants, such as, for example, cereals such as wheat, barley, sorghum and millet, rye, triticale, maize, rice or oats, and sugar cane. Especially preferred are Arabidopsis thaliana, Nicotiana tabacum, oilseed rape, soybean, corn (maize), wheat, linseed, potato and tagetes.

Plant organisms are furthermore, for the purposes of the invention, other organisms which are capable of photosynthetic activity, such as, for example, algae or cyanobacteria, and also mosses. Preferred algae are green algae, such as, for example, algae of the genus Haematococcus, Phaedactylum tricornatum, Volvox or Dunaliella.

Genetically modified plants according to the invention which can be consumed by humans or animals can also be used as food or feedstuffs, for example directly or following processing known in the art.

### 8. GENERATION OF THE PLANTS FOR THE METHOD OF THE INVENTION

Within the method of the invention it as an essential feature that two plants are crossed each of these comprising a specific DNA construct:
i) a first plant (the “endonuclease master plant”) comprising an expression cassette for expression of sequence specific DNA-endonuclease (the “endonuclease expression cassette”). Expression here is under the control of a parsley ubiquitin promoter as specified above.
ii) a second plant (the “trait plant”) comprising a recombination cassette for excision of a marker sequence and further comprising—optionally—an expression cassette for an agronomically valuable trait.

The individual features and preferred embodiments for the elements of said expression constructs or recombination cassettes are explained above in detail. The generation of the endonuclease master plant and the trait plant can be done by any of the multiple methods known in the art. The following procedures are only given by way of example.

### 8.1 Construction of Polynucleotide Constructs

Typically, DNA constructs (e.g., for an expression or recombination cassette) to be introduced into plants or plant cells are prepared using transgene expression techniques. Recombinant expression techniques involve the construction of recombinant nucleic acids and the expression of genes in transfected cells. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Vol. 152, Academic Press, hic., San Diego, Calif. (Berger); Current Protocols in Molecular Biology, F. M. Ausubel et al., eds., Current Protocols, a joint venture between Greene Publishing Associates, Inc. and John Wiley & Sons, Inc., (1998 Supplement), T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984). Preferably, the DNA constructs employed in the invention are generated by joining the abovementioned essential constituents of the DNA construct together in the abovementioned sequence using the recombination and cloning techniques with which the skilled worker is familiar.

Generally, a gene to be expressed will be present in an expression cassette, meaning that the gene is operably linked to expression control signals, e.g., promoters and terminators, that are functional in the host cell of interest. The genes that encode the sequence-specific DNA cleaving enzyme and, optionally, the selectable marker, will also be under the control of such signals that are functional in the host cell. Control of expression is most easily achieved by selection of a promoter. The transcription terminator is not generally as critical and a variety of known elements may be used so long as they are recognized by the cell. The invention contemplates polynucleotides operably linked to a promoter in the sense or antisense orientation.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress™ Expression System, Qiagen). The isolated and purified plasmids can then be further manipulated to produce other plasmids, used to transfect cells or incorporated into Agrobacterium tumefaciens to infect and transform plants. Where Agrobacterium is the means of transformation, shuttle vectors are constructed.

However, the skilled worker is aware that he may also obtain the DNA construct according to the invention in other ways. Thus, the host organism may already comprise one or more of the essential components of a DNA construct. A DNA construct is then generated by introducing one further, or more, essential components of said DNA construct in the correct position relative to the existing components in said organism. Thus, for example, the starting organism may already comprise one of the homology sequences (e.g., A or A′). If the organism already comprises a homology sequence A, introducing a DNA construct comprising all other elements (beside A) into the genomic DNA in proximity to the already existing homology sequence A gives rise to a DNA construct according to the invention.

Furthermore, the skilled worker is familiar with various ways in which the DNA construct according to the invention may be introduced into the genome of a host cell or organism. In this context, the insertion may be directed (i.e. taking place at a defined insertion site) or undirected (i.e. taking place randomly). Suitable techniques are known to the skilled worker and described by way of example herein below.

### 8.2 Methods for Introducing Constructs into Target Cells

A DNA construct employed in the invention may advantageously be introduced into cells using vectors into which said DNA construct is inserted. Examples of vectors may be plasmids, cosmids, phages, viruses, retroviruses or agrobacteria. In an advantageous embodiment, the expression cassette is introduced by means of plasmid vectors. Preferred vectors are those which enable the stable integration of the expression cassette into the host genome.

A DNA construct can be introduced into the target plant cells and/or organisms by any of the several means known to those of skill in the art, a procedure which is termed transformation (see also Keown et al. (1990) Meth Enzymol 185:527-537). For instance, the DNA constructs can be introduced into cells, either in culture or in the or gans of a plant by a variety of conventional techniques. For example, the DNA constructs can be introduced directly to plant cells using ballistic methods, such as DNA particle bombardment, or the DNA construct can be introduced using techniques such as electroporation and microinjection of cell. Particle-mediated transformation techniques (also known as “biolistics”) are described in, e.g., Klein et al. (1987) Nature 327:70-73; Vasil V et al. (1993) Bio/Technol 11:1553-1558; and Becker D et al. (1994) Plant J 5:299-307. These methods involve penetration of cells by small particles with the nucleic acid either within the matrix of small beads or particles, or on the surface. The biolistic PDS-1000 Gene Gun (Biorad, Hercules, Calif.) uses helium pressure to α-celerate DNA-coated gold or tungsten microcarriers toward target cells. The process is applicable to a wide range of tissues and cells from organisms, including plants. Other transformation methods are also known to those of skill in the art.

Microinjection techniques are known in the art and are well described in the scientific and patent literature. Also, the cell can be permeabilized chemically, for example using polyethylene glycol, so that the DNA can enter the cell by diffusion. The DNA can also be introduced by protoplast fusion with other DNA-containing units such as minicells, cells, lysosomes or liposomes. The introduction of DNA constructs using polyethylene glycol (PEG) precipitation is described in Paszkowski et al. (1984) EMBO J. 3:2717. Liposome-based gene delivery is e.g., described in WO 93/24640; Mannino and Gould-Fogerite (1988) BioTechniques 6(7):682-691; U.S. Pat. No. 5,279,833; WO 91/06309; and Felgner et al. (1987) Proc Natl Acad Sci USA 84:7413-7414).

Another suitable method of introducing DNA is electroporation, where the cells are permeabilized reversibly by an electrical pulse. Electroporation techniques are described in Fromm et al., (1985) Proc Natl Acad Sci USA 82:5824. PEG-mediated transformation and electroporation of plant protoplasts are also discussed in Lazzeri P (1995) Methods Mol Biol 49:95-106. Preferred general methods which may be mentioned are the calcium-phosphate-mediated transfection, the DEAE-dextran-mediated transfection, the cationic lipid-mediated transfection, electroporation, transduction and infection. Such methods are known to the skilled worker and described, for example, in Davis et al., Basic Methods In Molecular Biology (1986). For a review of gene transfer methods for plant and cell cultures, see, Fisk et al. (1993) Scientia Horticulturae 55:5-36 and Potrykus (1990) CIBA Found Symp 154:198.

Methods are known for introduction and expression of heterologous genes in both monocot and dicot plants. See, e.g., U.S. Pat. No. 5,633,446, U.S. Pat. No. 5,317,096, U.S. Pat. No. 5,689,052, U.S. Pat. No. 5,159,135, and U.S. Pat. No. 5,679,558; Weising et al. (1988) Ann. Rev. Genet. 22: 421-477. Transformation of monocots in particular can use various techniques including electroporation (e.g., Shimamoto et al. (1992) Nature 338:274-276; biolistics (e.g., EP-A1 270, 356); and Agrobacterium (e.g., Bytebier et al. (1987) Proc Natl Acad Sci USA 84:5345-5349).

In plants, methods for transforming and regenerating plants from plant tissues or plant cells with which the skilled worker is familiar are exploited for transient or stable transformation. Suitable methods are especially protoplast transformation by means of polyethylene-glycol-induced DNA uptake, biolistic methods such as the gene gun (“particle bombardment” method), electroporation, the incubation of dry embryos in DNA-containing solution, sonication and microinjection, and the transformation of intact cells or tissues by micro- or macroinjection into tissues or embryos, tissue electroporation, or vacuum infiltration of seeds. In the case of injection or electroporation of DNA into plant cells, the plasmid used does not need to meet any particular requirement. Simple plasmids such as those of the pUC series may be used. If intact plants are to be regenerated from the transformed cells, the presence of an additional selectable marker gene on the plasmid is useful.

In addition to these “direct” transformation techniques, transformation can also be carried out by bacterial infection by means of Agrobacterium tumefaciens or Agrobacterium rhizogenes. These strains contain a plasmid (Ti or Ri plasmid). Part of this plasmid, termed T-DNA (transferred DNA), is transferred to the plant following Agrobacterium infection and integrated into the genome of the plant cell.

For Agrobacterium-mediated transformation of plants, a DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-mediated transformation techniques are well described in the scientific literature. See, for example, Horsch et al. (1984) Science 233:496-498, Fraley et al. (1983) Proc Natl Acad Sci USA 80:4803-4807, Hooykaas (1989) Plant Mol Biol 13:327-336, Horsch R B (1986) Proc Natl Acad Sci USA 83(8):2571-2575), Bevans et al. (1983) Nature 304:184-187, Bechtold et al. (1993) Comptes Rendus De L'Academie Des Sciences Serie III-Sciences De La Vie-Life Sciences 316:1194-1199, Valvekens et al. (1988) Proc Natl Acad Sci USA 85:5536-5540.

A DNA construct of the invention is preferably integrated into specific plasmids, either into a shuttle, or intermediate, vector or into a binary vector). If, for example, a Ti or R1 plasmid is to be used for the transformation, at least the right border, but in most cases the right and the left border, of the Ti or Ri plasmid T-DNA is linked with the expression cassette to be introduced as a flanking region. Binary vectors are preferably used. Binary vectors are capable of replication both in E. coli and in Agrobacterium. As a rule, they contain a selection marker gene and a linker or polylinker flanked by the right or left T-DNA flanking sequence. They can be transformed directly into Agrobacterium (Holsters et al. (1978) Mol Gen Genet. 163:181-187). The selection marker gene permits the selection of transformed agrobacteria and is, for example, the nptII gene, which imparts resistance to kanamycin. The Agrobacterium, which acts as host organism in this case, should already contain a plasmid with the vir region. The latter is required for transferring the T-DNA to the plant cell. An Agrobacterium thus transformed can be used for transforming plant cells.

Many strains of Agrobacterium tumefaciens are capable of transferring genetic material—for example a DNA constructs according to the invention—, such as, for example, the strains EHA101(pEHA101) (Hood E E et al. (1996) J Bacteriol 168(3):1291-1301), EHA105(pEHA105) (Hood et al. 1993, Transgenic Research 2, 208-218), LBA4404(pAL4404) (Hoekema et al., (1983) Nature 303:179-181), C58C1 (pMP90) (Koncz and Schell (1986) Mol Gen Genet. 204, 383-396) and C58C1 (pGV2260) (Deblaere et al. (1985) Nucl Acids Res. 13, 4777-4788).

The agrobacterial strain employed for the transformation comprises, in addition to its disarmed Ti plasmid, a binary plasmid with the T-DNA to be transferred, which, as a rule, comprises a gene for the selection of the transformed cells and the gene to be transferred. Both genes must be equipped with transcriptional and translational initiation and termination signals. The binary plasmid can be transferred into the agrobacterial strain for example by electroporation or other transformation methods (Mozo & Hooykaas (1991) Plant Mol Biol 16:917-918). Coculture of the plant explants with the agrobacterial strain is usually performed for two to three days.

A variety of vectors could, or can, be used. In principle, one differentiates between those vectors which can be employed for the Agrobacterium-mediated transformation or agroinfection, i.e. which comprise a DNA construct of the invention within a T-DNA, which indeed permits stable integration of the T-DNA into the plant genome. Moreover, border-sequence-free vectors may be employed, which can be transformed into the plant cells for example by particle bombardment, where they can lead both to transient and to stable expression.

The use of T-DNA for the transformation of plant cells has been studied and described intensively (EP-A1 120 516; Hoekema, In: The Binary Plant Vector System, Offsetdrukkerij Kanters B. V., Alblasserdam, Chapter V; Fraley et al. (1985) Crit. Rev Plant Sci 4:1-45 and An et al. (1985) EMBO J. 4:277-287). Various binary vectors are known, some of which are commercially available such as, for example, pBIN19 (Clontech Laboratories, Inc. USA).

To transfer the DNA to the plant cell, plant explants are cocultured with Agrobacterium tumefaciens or Agrobacterium rhizogenes. Starting from infected plant material (for example leaf, root or stalk sections, but also protoplasts or suspensions of plant cells), intact plants can be regenerated using a suitable medium which may contain, for example, antibiotics or biocides for selecting transformed cells. The plants obtained can then be screened for the presence of the DNA introduced, in this case a DNA construct according to the invention. As soon as the DNA has integrated into the host genome, the genotype in question is, as a rule, stable and the insertion in question is also found in the subsequent generations. As a rule, the expression cassette integrated contains a selection marker which confers a resistance to a biocide (for example a herbicide) or an antibiotic such as kanamycin, G 418, bleomycin, hygromycin or phosphinotricin and the like to the transformed plant. The selection marker permits the selection of trans-formed cells (McCormick et al., Plant Cell Reports 5 (1986), 81-84). The plants obtained can be cultured and hybridized in the customary fashion. Two or more generations should be grown in order to ensure that the genomic integration is stable and hereditary.

The abovementioned methods are described, for example, in B. Jenes et al., Techniques for Gene Transfer, in: Transgenic Plants, Vol. 1, Engineering and Utilization, edited by S D Kung and R Wu, Academic Press (1993), 128-143 and in Potrykus (1991) Annu Rev Plant Physiol Plant Molec Biol 42:205-225). The construct to be expressed is preferably cloned into a vector which is suitable for the transformation of Agrobacterium tumefaciens, for example pBin19 (Bevan et al. (1984) Nucl Acids Res 12:8711).

The DNA construct of the invention can be used to confer desired traits on essentially any plant. One of skill will recognize that after DNA construct is stably incorporated in transgenic plants and confirmed to be operable, it can be introduced into other plants by sexual crossing. Any of a number of standard breeding techniques can be used, depending upon the species to be crossed.

### 8.3 Regeneration of Transgenic Plants

Transformed cells, i.e. those which comprise the DNA integrated into the DNA of the host cell, can be selected from untransformed cells if a selectable marker is part of the DNA introduced. A marker can be, for example, any gene which is capable of conferring a resistance to antibiotics or herbicides (for examples see above). Transformed cells which express such a marker gene are capable of surviving in the presence of concentrations of a suitable antibiotic or herbicide which kill an untransformed wild type. As soon as a transformed plant cell has been generated, an intact plant can be obtained using methods known to the skilled worker. For example, callus cultures are used as starting material. The formation of shoot and root can be induced in this as yet undifferentiated cell biomass in the known fashion. The shoots obtained can be planted and cultured.

Transformed plant cells, derived by any of the above transformation techniques, can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124176, Macmillian Publishing Company, New York (1983); and in Binding, Regeneration of Plants, Plant Protoplasts, pp. 21-73, CRC Press, Boca Raton, (1985). Regeneration can also be obtained from plant callus, explants, somatic embryos (Dandekar et al. (1989) J Tissue Cult Meth 12:145; McGranahan et al. (1990) Plant Cell Rep 8:512), organs, or parts thereof. Such regeneration techniques are described generally in Klee et al. (1987) Ann Rev Plant Physiol 38:467-486.

### 9. CROSSING OF THE TRAIT PLANT AND THE ENDONUCLEASE MASTER PLANT AND GENERATION OF MARKER-FREE DESCENDANTS

After transformation, selection and regeneration of transgenic trait plants and endonuclease master plants, these plants can be further bred (e.g. to homozygous plants by selfing or crossing into elite germplasm). The trait plants and/or endonuclease master plants to be employed in the method of the invention may comprise one or more copies of the respective DNA construct introduced into their genome and may be homozygous or heterozygous with respect to said recombination cassette or endonuclease expression cassette, respectively.

Selfing and/or crossing of the trait plant with the endonuclease marker plant can be done by any procedure known in the art. To reduce the possibility of self-pollination during crossing, the flowers from the female parent may be used before the anthers begin to shed pollen onto the stigma. For the male parent, an open flower that is visibly shedding pollen should be chosen. The appearance of flowers at the appropriate developmental stage varies among plant species, cultivars and growth conditions.

After the fertilization process, F1 seeds are harvested, germinated and grown into mature plants. Plants from which the selectable marker of the “trait construct” is removed from all cells may be obtained in the first generation (F1) or in the second (F2) or later (Fn) generation. Isolation and identification of descendants which underwent an excision process can be done at any stage of plant development. Methods for said identification are well known in the art and may comprise—for example —PCR analysis, Northern blot, Southern blot, or phenotypic screening (e.g., for an negative selection marker; compare examples 8c and 8d).

Descendants of the F1 plants may be obtained by sexual propagation as outlined above. They may also be obtained by asexual propagation. For the latter tissue culture procedures may be applied. Asexual propagation is especially preferred in cases, where plants are not yet homogenously consisting of cells which all have undergone successful sequence excision. For asexual propagation tissues from plants (which may be chimeric for the recombination event, i.e. the excision did not take place in all cells) are used as explants to regenerate new plants. Since these new plants may be regenerated from a single cell, all cells of this asexually obtained descendant are identical regarding the excision event to the original cell. By selecting for plants which originate from a cell, in which the desired recombination event (e.g. excision of a selectable marker) occurred, plants may be obtained which have the recombination event present in all cells. A very efficient method to regenerate whole plants from single cells may be applied by making a slurry of the explant (see example 8c where a detailed method for efficient regeneration of rapeseed plants is disclosed as an example).

Descendants may comprise the construct encoding the sequence specific endonuclease (optionally together with a selectable marker on the same construct). These cassettes are preferably removed by segregation in the progeny (sexual propagation). This might, for example, be achieved by selfing or crossing to a non-transgenic wildtype plant.

Descendants may comprise one or more copies of the agronomically valuable trait gene. Preferably, descendants are isolated which only comprise one copy of said trait gene. It is another inventive feature of the present invention that multiple insertion (e.g., of a T-DNA) in one genomic location will be reduced to a single insertion event by excision of the redundant copies together with the marker gene of the remaining copy (FIG. 10).

In a preferred embodiment the transgenic plant made by the process of the invention is marker-free. The terms “marker-free” or “selection marker free” as used herein with respect to a cell or an organisms are intended to mean a cell or an organism which is not able to express a functional selection marker protein (encoded by expression cassette b1; as defined above) which was inserted into said cell or organism in combination with the gene encoding for the agronomically valuable trait. The sequence encoding said selection marker protein may be absent in part or—preferably—entirely. Furthermore the promoter operably linked thereto may be dysfunctional by being absent in part or entirely.

The resulting plant may however comprise other sequences which may function as a selection marker. For example the plant may comprise as a agronomically valuable trait a herbicide resistance conferring gene or the endonuclease expression cassette may be linked to a selection marker. However, it is most preferred that the resulting plant does not comprise any selection marker.

### 10. COMBINATION WITH OTHER RECOMBINATION ENHANCING TECHNIQUES

In a further preferred embodiment, the efficacy of the recombination system is increased by combination with systems which promote homologous recombination. Such systems are described and encompass, for example, the expression of proteins such as RecA or the treatment with PARP inhibitors. It has been demonstrated that the intrachromosomal homologous recombination in tobacco plants can be increased by using PARP inhibitors (Puchta H et al. (1995) Plant J. 7:203-210). Using these inhibitors, the homologous recombination rate in the recombination cassette after induction of the sequence-specific DNA double-strand break, and thus the efficacy of the deletion of the transgene sequences, can be increased further. Various PARP inhibitors may be employed for this purpose. Preferably encompassed are inhibitors such as 3-aminobenzamide, 8-hydroxy-2-methylquinazolin-4-one (NU1025), 1,11 b-dihydro-(2H)benzopyrano(4,3,2-de)isoquinolin-3-one (GPI 6150), 5-aminoisoquino-linone, 3,4-dihydro-5-(4-(1-piperidinyl)butoxy)-1(2H)-isoquinolinone, or the compounds described in WO 00/26192, WO 00/29384, WO 00/32579, WO 00/64878, WO 00/68206, WO 00/67734, WO 01/23386 and WO 01/23390.

In addition, it was possible to increase the frequency of various homologous recombination reactions in plants by expressing the E. coli RecA gene (Reiss B et al. (1996) Proc Natl Acad Sci USA 93(7):3094-3098). Also, the presence of the protein shifts the ratio between homologous and illegitimate DSB repair in favor of homologous repair (Reiss B et al. (2000) Proc Natl Acad Sci USA 97(7):3358-3363). Reference may also be made to the methods described in WO 97/08331 for increasing the homologous recombination in plants. A further increase in the efficacy of the recombination system might be achieved by the simultaneous expression of the RecA gene or other genes which increase the homologous recombination efficacy (Shalev G et al. (1999) Proc Natl Acad Sci USA 96(13):7398-402). The above-stated systems for promoting homologous recombination can also be advantageously employed in cases where the recombination construct is to be introduced in a site-directed fashion into the genome of a eukaryotic organism by means of homologous recombination.

### 11. PREFERRED COMBINATIONS

### 11.1 Basic Principle (see FIGS. 1 and 2)

In a preferred embodiment the DNA construct in the first plant (the trait plant, TP) comprises an expression cassette for a negative selections marker (ENS) under control of a promoter and a transcription terminator. In addition, the DNA construct comprises two (or more) recognition sequences (S1 and S2) flanking the selection marker expression cassette in a way that cleavage at this two recognition sequences excises said cassette. These two recognition sequences are flanked by the two homology sequences A and A′. Preferably, the distance between a homology sequences (A and A′, respectively) and a recognition sequence (e.g., S1 or S2) is less than 100 base pairs, preferably less than 50 base pairs, more preferably less than 25 base pairs, most preferably the recognition sequence is attached directly to the end of the homology sequence. Furthermore the DNA construct in the second plant (the endonuclease master plant, EMP) comprises a second expression cassette for a sequence specific endonuclease (EE) under control of a parsley ubiquitin promoter and a transcription terminator.

After crossing (X) of the two plants (symbolized by the boxes), the resulting descendant is grown (G) and optionally further propagated into following generation(s). Cleavage (C) at the recognition sequences (S1 and S2) is induced when the parsley ubiquitin promoter EP becomes active and causes expression of the sequence specific endonuclease. By generation of the double-strand breaks homologous recombination (HR) is induced between the homology sequences A and A′. The endonuclease may be separated from the trait by further crossing and segregation (S).

In an preferred embodiment the DNA construct in the trait plant comprises a second expression cassette (e.g., encoding an agronomically valuable trait) outside of the region flanked by the homology sequences, which is therefore not excised from the genome by the homologous recombination reaction (see. FIG. 2). Furthermore the DNA construct may comprise a fourth expression cassette for a counter-selection marker, preferably localized between the homology sequences A and A′ (FIG. 9).

### 11.2 Variations where the Homology Sequences are Part of the Expression Cassettes (See FIG. 3 to 5)

In another preferred embodiment the homology sequences (A and A′) are part of the expression cassettes of the DNA construct in the trait plant. Any part of the expression cassettes may be suitable to function as a homology sequence. Preferably, the homology sequences are identical with the promoter regions (FIG. 3) or the terminator region (FIG. 4). Homologous recombination between these functional elements preferably reconstitutes the expression cassette for the agronomically valuable trait but excises the expression cassettes for the selection marker. It is possible that the homology region does not only comprises promoter or terminator regions but extents into the coding region (e.g., of the gene encoding the agronomically valuable trait (FIG. 5).

### 11.3 DNA Constructs Comprising Only One Recognition/Cleavage Site (FIG. 6-8)

The DNA construct of the invention may comprise only one recognition/cleavage sequence for the sequence specific endonuclease. Preferably this sequence is localized close to one of the homology sequences and is within the region flanked by said homology sequences. Various locations are possible (FIG. 6-8).

12. Sequences

| 1. SEQ ID NO: 1 | Nucleic acid sequence coding for Construct I | | |
|---|---|---|---|
| | Features of the T-DNA: | | |
| | Position 6 to 151 | | right border |
| | Position 152 to 161 | | N, region encoding different expression |
| | | | cassettes for the sequence specific |
| | | | DNA-endonuclease |
| | Position 1681 to 198 | (compl.) | nosP::nptII::nosT cassette (with nptII |
| | | | ORF from 1324 to 546, compl.) |
| | Position 1694 to 1908 | | left border |
| | Rest of the plasmid is pSUN backbone (including aadA bacterial resis- | | |
| | tance gene encoded at position 7279 to 6488, complementary) | | |
| 2. SEQ ID NO: 2 | Nucleic acid sequence coding for insert of pCB603-100 | | |
| | Features | | |
| | Position 249 to 43 | (compl.) | ocs terminator |
| | Position 1010 to 303 | (compl.) | ORF encoding I-Scel |
| | Position 2790 to 1014 | (compl.) | fragment of promoter AtSERK1 |
| | | | (incl. 5′-UTR) |
| 3. SEQ ID NO: 3 | Nucleic acid sequence coding for insert of pCB622-3 | | |
| | Features | | |
| | Position 249 to 43 | (compl.) | ocs terminator |
| | Position 1011 to 304 | (compl.) | ORF encoding I-Scel |
| | Position 2168 to 1014 | (compl.) | fragment of promoter Atcyc1 (incl. 5′- |
| | | | UTR) |
| 4. SEQ ID NO: 4 | Nucleic acid sequence coding for insert of pCB653-37 | | |
| | Features | | |
| | Position 249 to 43 | (compl.) | ocs terminator |
| | Position 1011 to 304 | (compl.) | ORF encoding I-Scel |
| | Position 2201 to 1013 | (compl.) | fragment of promoter erecta (incl. 5′- |
| | | | UTR) |
| 5. SEQ ID NO: 5 | Nucleic acid sequence coding for insert of pCB652-124 | | |
| | Features | | |
| | Position 249 to 43 | (compl.) | ocs terminator |
| | Position 1011 to 304 | (compl.) | ORF encoding I-Scel |
| | Position 2956 to 1059 | (compl.) | fragment of promoter invGF (incl. 5′- |
| | | | UTR) |
| 6. SEQ ID NO: 6 | Nucleic acid sequence coding for insert of pCB632-17 | | |
| | Features | | |
| | Position 90 to 1407 | | STPT promoter |
| | Position 1491 to 2198 | | ORF encoding I-Scel |
| | Position 2282 to 2516 | | CatDpA terminator |
| 7. SEQ ID NO: 7 | Amino acid sequence coding for I-Scel | | |
| 8. SEQ ID NO: 8 | Nucleic acid sequence coding for parsley (Petroselinum crispum) | | |
| | ubiquitin promoter | | |
| 9. SEQ ID NO: 9 | Nucleic acid sequence coding for binary vector pCB666-3 | | |
| | Features of T-DNA | | |
| | Position 3636 to 3850 | | Left Border |
| | Position 3630 to 2313 | (compl) | STPT promoter from Arabidopsis |
| | Position 2289 to 277 | (compl) | AHAS resistance gene from Arabidopsis; |
| | | | encodes the S653N mutation |
| | | | conferring resistance towards imidazoline |
| | | | herbicides |
| | Position 260 to 8 | (compl) | nos terminator |
| | Position 12807 to 11606 | (compl) | sequence derived from Arabidopsis |
| | | | downstream of AHAS coding region |
| | Position 11582 to 10600 | (compl) | Parsley ubiquitin promoter |
| | | | /5′UTR with intron |
| | Position 10582 to 9875 | (compl) | sequence encoding I-Scel |
| | Position 9599 to 9851 | (compl) | nos terminator |
| | Position 9395 to 9540 | | Right Border |
| 10. SEQ ID NO: 10 | Nucleic acid sequence coding for binary vector pCB657-41 | | |
| | Features of T-DNA | | |
| | Position 6976to 7192 | | Left Border |
| | Position 6904 to 5587 | (compl) | STPT promoter from Arabidopsis |
| | Position 5533 to 3536 | (compl) | GUS gene; |
| | | | PIV2 intron at 5148 to 4960 (compl) |
| | Position 3461 to 3257 | (compl) | 35S terminator |
| | Position 3182 to 3199 | | I-Scel site |
| | Position 3200 to 3229 | | I-Crel site |
| | Position 3167 to 1356 | (compl) | A. thaliana nitrilase 1 promoter |
| | Position 1312 to 509 | (compl) | nptII gene |
| | Position 462 to 445 | (compl) | I-Scel site |
| | Position 444 to 415 | (compl) | I-Crel site |
| | Position 38 to 183 | | Right Border |
| 11. SEQ ID NO: 11 | Nucleic acid sequence coding for binary vector JB010qcz | | |
| | Features of T-DNA | | |
| | Position 363 to 149 | (compl) | Left Border |
| | Position 372 to 6088 | | AHAS expression cassette¹ |
| | Position 6106 to 7088 | | PcUbi promoter |
| | | | (including an intron in the 5′UTR) |
| | Position 7106 to 7813 | | I-Scel coding region |
| | Position 7837 to 8089 | | nos terminator |
| | Position 8148 to 8293 | | Right Border |
| 12. SEQ ID NO: 12 | Nucleic acid sequence coding for binary vector pCB583-40 | | |
| | Features of T-DNA | | |
| | Position 8 to 153 | | Right Border |
| | Position 419 to 244 | (compl) | nos terminator |
| | Position 3144 to 525 | (compl) | overlapping, non-functional helves of |
| | | | GUS gene with an I-Scel and I-Crel |
| | | | site inbetween |
| | Position 3620 to 3251 | (compl) | 35S promoter |
| | Position 4041 to 3786 | (compl) | nos terminator |
| | Position 4663 to 4112 | (compl) | pat gene conferring BASTA |
| | | | herbicide resistance |
| | Position 4999 to 4669 | (compl) | nos promoter |
| | Position 5015 to 5228 | | Left Border |
| 13. SEQ ID NO: 13 | Nucleic acid sequence coding for binary vector pRS8 | | |
| | Features of T-DNA | | |
| | Position 8 to 153 | | Right Border |
| | Position 490 to 252 | (compl) | ocs terminator |
| | Position 1208 to 501 | | I-Scel CDS |
| | Position 1809 to 1280 | (compl) | 35S promoter |
| | Position 2111 to 1856 | (compl) | nos terminator |
| | Position 2982 to 2204 | (compl) | nptII gene conferring kanamycin |
| | | | resistance |
| | Position 3339 to 3003 | (compl) | nos promoter |
| | Position 3566 to 3352 | | Left Border |

| | |
|---|---|
| 14. SEQ ID NO: 14 | I-Scel coding region interrupted by an intron |
| | I-Scel coding sequence comprising the potato PIV2 intron at position 565 to 753 |
| 15. SEQ ID NO: 15 | Nucleic acid sequence coding for parsley (Petroselinum crispum) |
| | ubiquitin promoter (alternative form) |

¹The AHAS ORF (position 2855 to 4867) from Arabidopsis encodes the S653N mutation conferring resistance towards imidazoline herbicides

### 13. FIGURES

The following abbreviations apply to the figures in general:
A: Homology sequence A
A′: Homology sequence A′
A/A′: Sequence as the result of homologous recombination between A and A′
C: Sequence specific cleavage
CS: Counter selection marker
E: Sequence encoding sequence specific DNA-endonuclease
EE: Complete expression cassette for endonuclease
EN: Complete expression cassette for further nucleic acid sequence (coding for e.g., agronomically valuable trait)
PP: Parsley ubiquitin promoter
ENS: Complete expression cassette for negative selection marker
I: Insertion into the genome (e.g., chromosomal DNA) G Growing of plants and—optionally—generation of subsequent generation
HR: Homologous recombination
N: Further nucleic acid sequence (coding for e.g., agronomically valuable trait)
NS: Negative selection marker
NU: Endonuclease
Pₙ: Promoter
Sₙ: Recognition sequence for the site-directed induction of DNA double-strand breaks (e.g., S1: First recognition sequence). The recognition sequences may be different (e.g., functioning for different endonucleases) or—preferably—identical (but only placed in different locations).
Sₙ*; Part of recognition sequence Sₙ remaining after cleavage
Tₙ: Terminator sequence
RB/LB: Right/left border of Agrobacterium T-DNA
X: Crossing of plants
S: Segregation by crossing or selfing, may be monitored by e.g. PCR or Southern

FIG. 1: Basic Principle
The boxes represent the individual plants (EMP: Endonuclease master plant; TP trait plant). The DNA construct in the trait plant comprises:
   An expression cassette for a negative selection marker (ENS) under control of a first promoter and a transcription terminator,
   Two recognition sequences (S1 and S2) for the sequence specific endonuclease expressed by the second expression cassette, flanking the two expression cassettes in a way that cleavage at this two recognition sequences excises said cassettes, and
   Two homology sequences A and A′ flanking the two recognition sequences.
The DNA construct in the endonuclease master plant comprises:
   An expression cassette for a sequence specific endonuclease (NU) under control of an parsley ubiquitin promoter and a transcription terminator.
After crossing (X) of the two plants (symbolized by the boxes), the resulting descendants are grown (G) and optionally further propagated into following generation(s). Cleavage (C) at the recognition sequences (S1 and S2) is induced when the parsley ubiquitin promoter (PP) becomes active and causes expression of the sequence specific endonuclease (NU). By generation of the double-strand breaks, homologous recombination (HR) is induced between the homology sequences A and A′. The endonuclease may be separated from the trait by further crossing and segregation (S).

FIG. 2: Introduction of an Agronomically Valuable Trait
The DNA construct in the trait plant further comprises a second expression cassette (e.g., encoding an agronomically valuable trait (EN) under control of a promoter and a terminator) outside of the domain flanked by the homology sequences. (In this case the DNA construct was introduced into the chromosomal DNA by Agrobacterium mediated transformation. Therefore the inserted elements are flanked by right (RB) and left border (LB) of Agrobacterium T-DNA). Crossing (X), growing (G) occurs as described above. Cleavage (C) and the subsequent homologous recombination (HR) excises the expression cassettes for the selection marker. However, the expression cassette for the agronomically valuable trait is not excised but remains in the chromosomal DNA. The endonuclease may be separated from the trait by further crossing and segregation (S).

FIG. 3 Use of Promoter Sequences as Homology Sequences
(Only the cleavage and homologous recombination part of the method are shown)
The homology sequences (A and A′) are the promoters of the expression cassettes of the DNA construct (P1=A; P1=A′). Cleavage (C) and subsequent homologous recombination (HR) between these promoters reconstitutes the expression cassette for the agronomically valuable trait (N) but excises the expression cassettes for the negative selection marker (NS). In the present example DNA introduction was realized by Agrobacterium transformation and the inserted sequence is flanked by Agrobacterium left/right borders (other ways of introduction e.g., by particle bombardment are possible and would not require these borders).

FIG. 4 Use of Terminator Sequences as Homology Sequences
(Only the cleavage and homologous recombination part of the method are shown)
The homology sequences (A and A′) are the terminators of the expression cassettes of the DNA construct (T1=A; T1=A′). Cleavage (C) and subsequent homologous recombination (HR) between these terminators reconstitutes the expression cassette for the agronomically valuable trait (N) but excises the expression cassettes for the negative selection marker (NS). In the present example DNA introduction was realized by Agrobacterium transformation and the inserted sequence is flanked by Agrobacterium left/right borders (other ways of introduction e.g., by particle bombardment are possible and would not require these borders).

FIG. 5 Use of Part of the Excision Cassettes as Homology Sequences
(Only the cleavage and homologous recombination part of the method are shown)
The homology sequences (A and A′) are the part of the excision cassettes of the DNA construct (indicated by black bars below; A; A′). Cleavage (C) and subsequent homologous recombination (HR) between these terminators reconstitutes the expression cassette for the agronomically valuable trait (N) but excises the expression cassettes for the negative selection marker (NS). In the present example DNA introduction was realized by Agrobacterium transformation and the inserted sequence is flanked by Agrobacterium left/right borders (other ways of introduction e.g., by particle bombardment are possible and would not require these borders).

FIG. 6-8 DNA Constructs with One Recognition Sequence
(Only the cleavage and homologous recombination part of the method are shown)
The DNA construct of the invention may comprise only one recognition/cleavage sequence for the sequence specific endonuclease. Preferably this sequence is localized close to one of the homology sequences and is within the region flanked by said homology sequences. Various locations are possible (FIG. 6-8).

FIG. 9: DNA Construct Comprising a Counter-Selection Marker
(Only the cleavage and homologous recombination part of the method are shown)
The DNA construct in the trait plant may comprise:
   A first expression cassette for a negative selection marker (NS) under control of a promoter (P1) and a transcription terminator (T1),
   A second expression cassette for a counter-selection marker (CS) under control of a promoter (P2) and a transcription terminator (T2),
   Two recognition sequences (S1 and S2) for the sequence-specific endonuclease encoded by the second expression cassette, flanking the two expression cassettes in a way that cleavage at this two recognition sequences excises said cassettes,
   Two homology sequences A and A′ flanking the two recognition sequences, and
   A third expression cassette for an agronomically valuable trait (localized outside of the region flanked by the homology sequences A and A′) under control of a promoter (P3) and a transcription terminator (T4)
After crossing with the endonuclease master plant (as described for FIG. 1) and growing of the descendants, cleavage (C) at the recognition sequences (S1 and S2) is induced when the parsley ubiquitin promoter becomes active and causes expression of the sequence specific endonuclease. By generation of the double-strand breaks homologous recombination (HR) is induced between the homology sequences A and A′.

FIG. 10 Application of the method of the invention to simplify transformation events.
(Only the cleavage and homologous recombination part of the method are shown)
It is another inventive feature of the present invention that multiple insertion (e.g., of a T-DNA) in one genomic location will be reduced to a single insertion event by excision of the redundant copies. In the depicted case two copies of a T-DNA have inserted into the genome (box 1 and box 2). Both comprise the expression cassette for a negative selection marker (ENS) and an expression cassette for an agronomically valuable trait (EN). Cleavage and subsequent homologous recombination deletes the marker and the oblivious copy and the resulting event becomes undistinguishable from a single insertion event of which the selectable marker has been eliminated by homologous recombination (see FIG. 2 in comparison).

FIG. 11-12: Use of Endogenous Promoters

One or more expression cassette of the DNA construct may constituted after insertion into the genome by inserting the nucleic acid to be expressed (e.g., the negative selection marker (FIG. 11) or the gene encoding the agronomically valuable trait (FIG. 12)) under control of an endogenous promoter.

FIG. 13: Schematic representation of the pGU-sce-US construct before (A) and after (B) intrachromosomal homologous recombination between the identical homology sequences A and A′. The original construct (A) did not produce active GUS protein, while the arrangement obtained after intrachromosomal homologous recombination (hereinafter called ICHR) (B) restored an intact GUS gene and therefore produced active GUS protein. The cells/plants in which ICHR had taken place were identified by histochemical GUS staining.

FIG. 14: Leaves from plants (A/B) containing the pGU-Sce-US reporter construct. The only difference between these two plants is that plant (B) contained in addition an I-SceI expression cassette (pRS8).

FIG. 15-26: Vector Maps

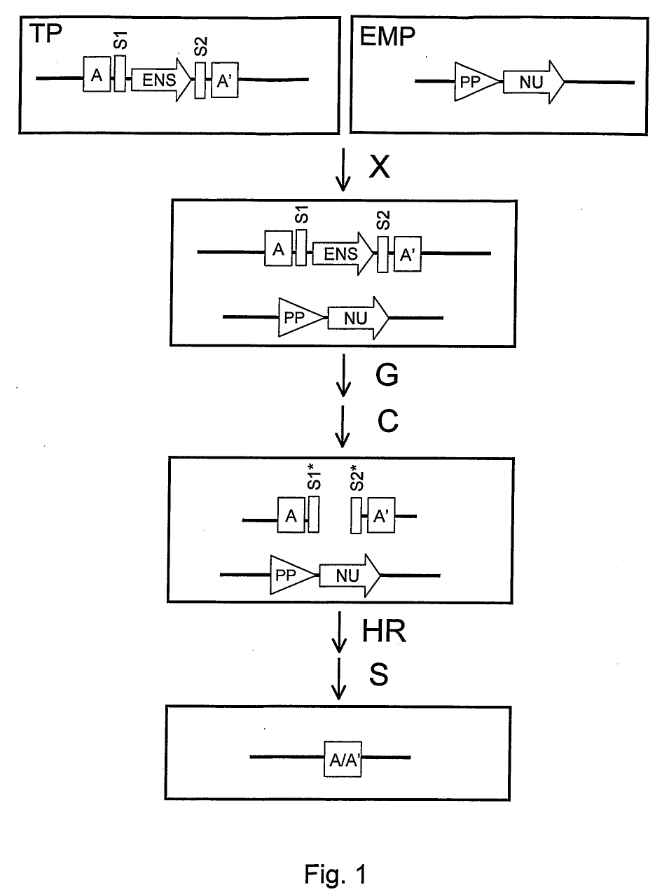

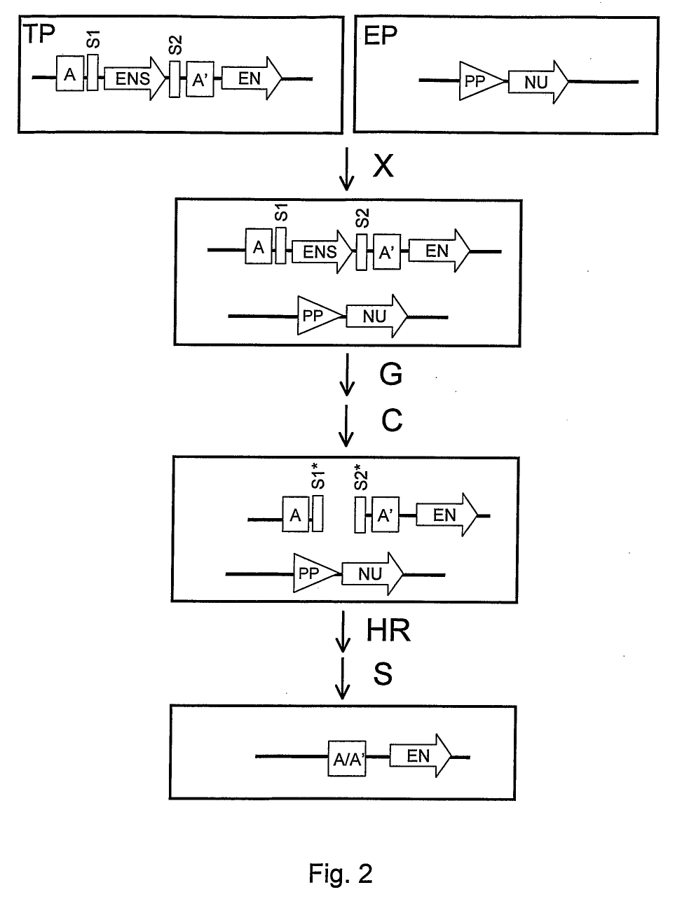

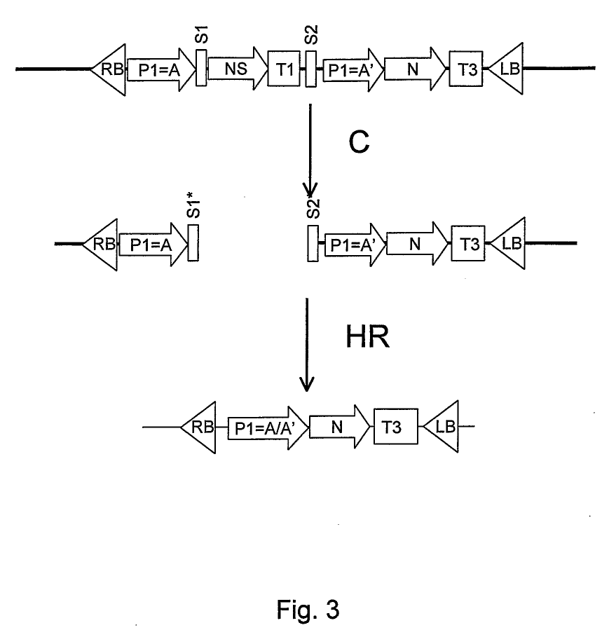

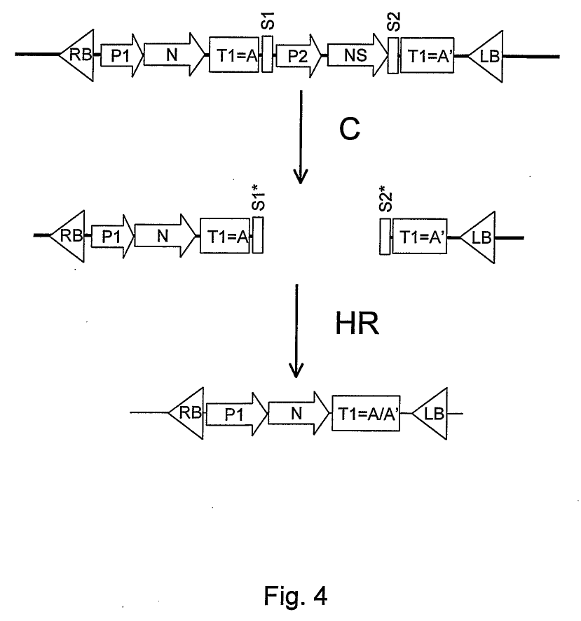

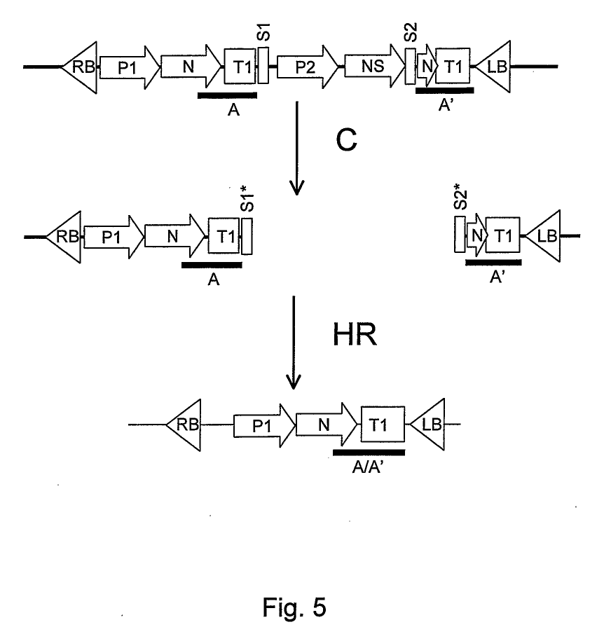

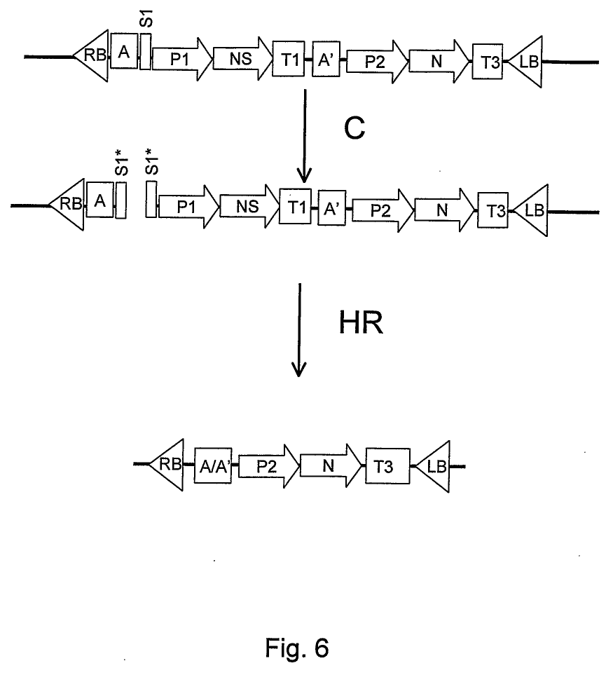

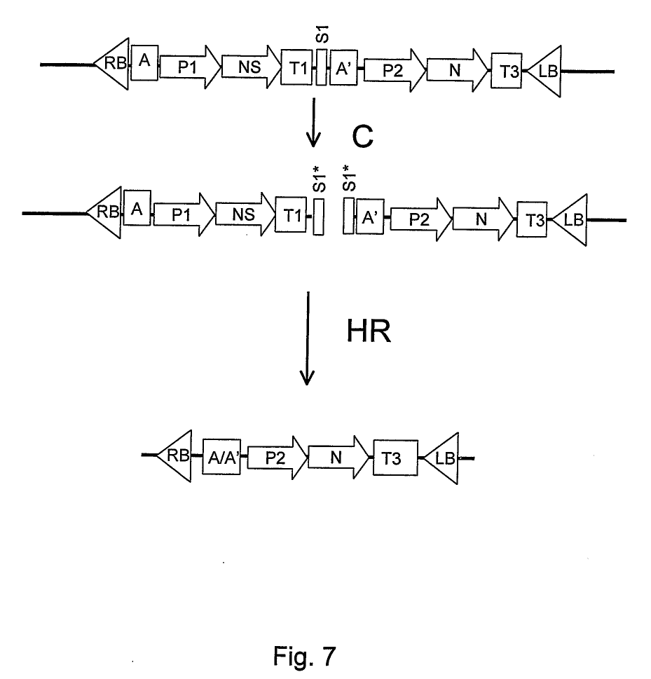

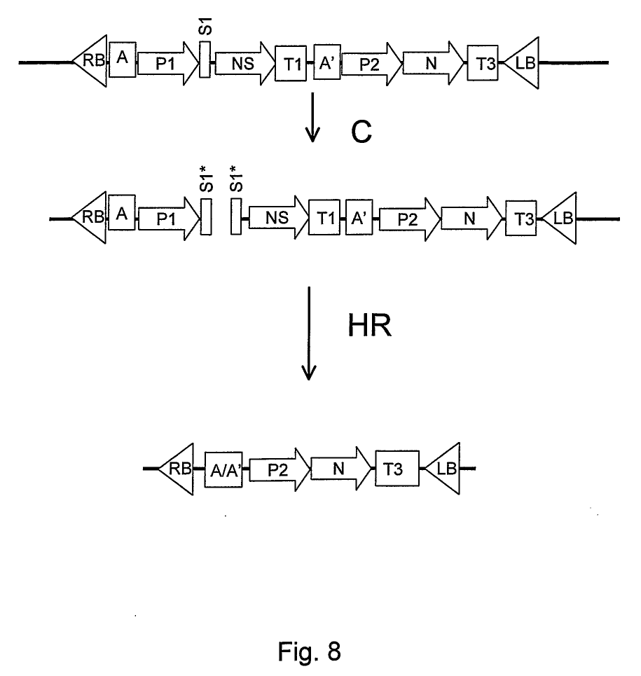

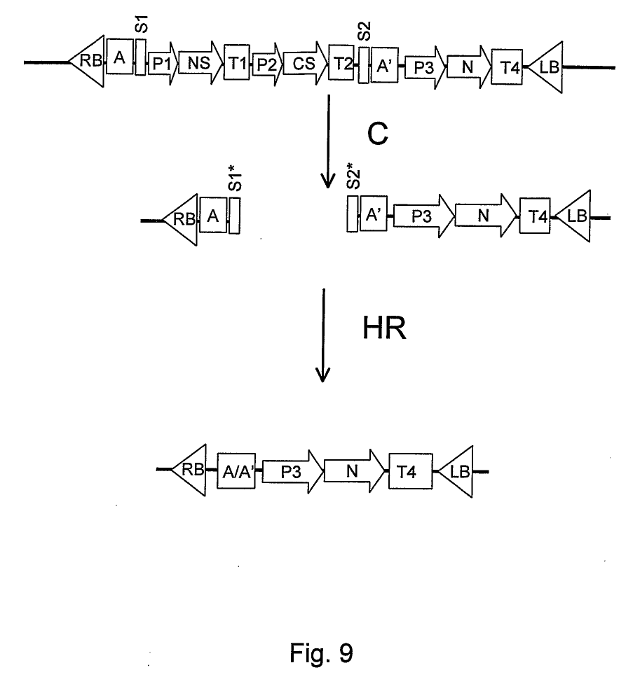

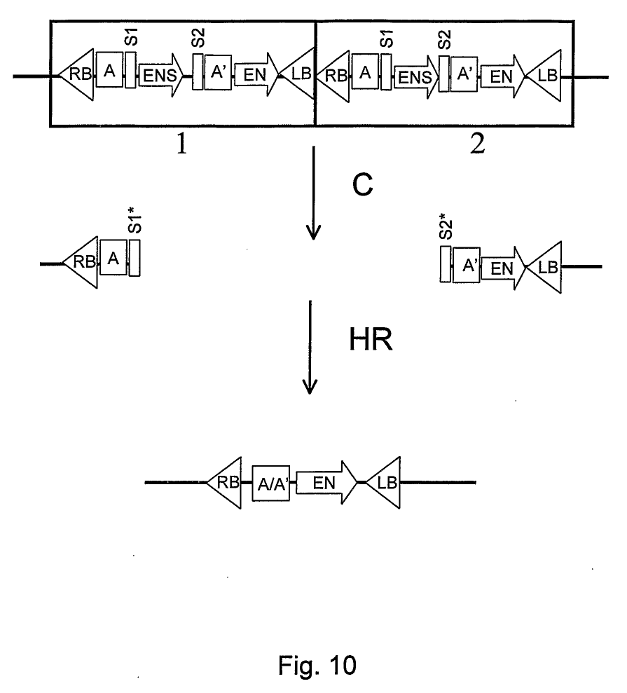

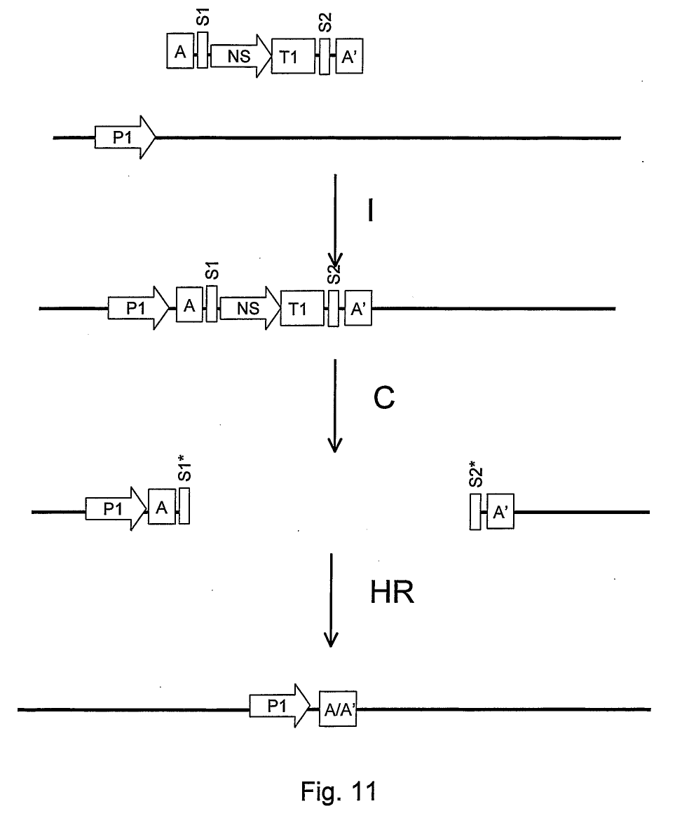

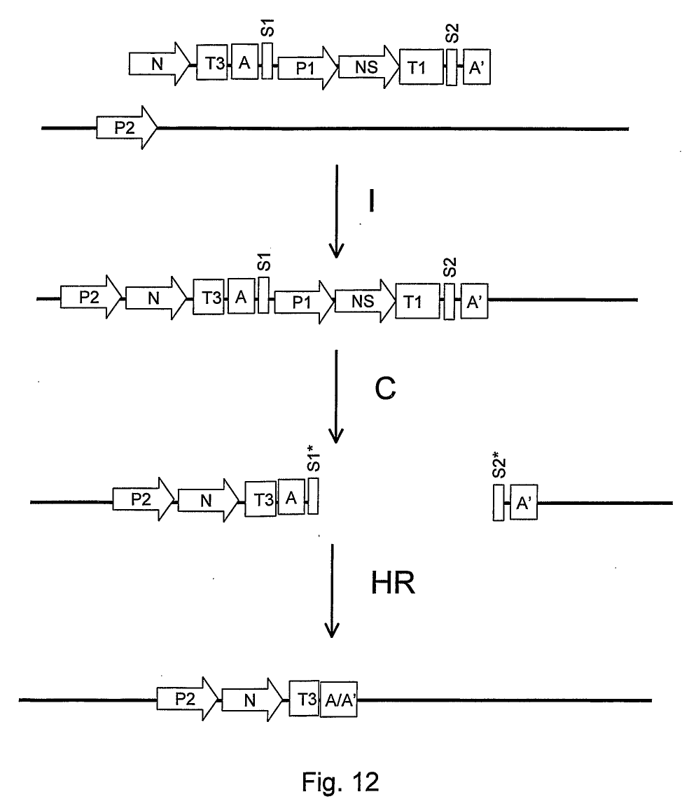

Vector Backbone Elements:
aadA: prokaryotic selectable marker conferring spectinomycin resistance
ColE1: origin of replication, e.g. for E. coli
repA/pVS1: elements for replication, e.g. in Agrobacterium

T-DNA Elements:
LB: Left border
RB: Right border
sTPT: constitutive plant promoter
GUS(int): uidA gene encoding GUS with an intron
GUS: uidA gene encoding GUS
35SpA: terminator sequence derived from CaMV 35S RNA encoding gene (duplicated, serves as homology region A and A′, respectively)
Nit1-P: constitutive plant promoter
nptII: eukaryotic selectable marker conferring kanamycin resistance
nosT: terminator sequence derived from nos gene from Agrobacterium
USP-P: USP promoter active in immature embryos
invGF promoter: invGF promoter active in pollen
Perecta Erecta promoter
Prom AtSERK1 AtSERK1 promoterAtcyc1A Prom Atcyc1A promoter
I-SceI: gene encoding the homing endonuclease I-SceI
I-SceI RC recognition sequence for I-SceI homing endonuclease
I-CreI RC recognition sequence for I-SceI homing endonuclease
I-CreI Exon1/2: Two artificial exons of the I-CreI gene T-CatD terminator sequence
35SpA terminator sequence
nosT terminator sequence
NNNNNNNNNN region encoding different expression cassettes for the sequence specific DNA-endonuclease (the number of Ns is only symbolic, the insert at this place can have any length)

FIG. 27 Seedling and leafs from seedlings obtained from crosses between GU-US reporter lines (construct pCB583-40) and sTPT::I-SceI (construct pCB632-17; based on the constitutive sTPT promoter; panel “A”) and PcUbi::I-SceI (construct JB010cqz, based on the parsley ubiquitin promoter; panel “B”), respectively, after histochemical GUS staining. A significant more intense staining (indicated by dark areas) can be observed for the parsley based promoter construct in panel “B” indicating that more cells harbor the recombination event, i.e. the functional GUS gene in this particular example.

FIG. 28 Schematic representation of the procedure to obtain full blue plants (equivalent to full marker-free plants) from plants with blue spots (chimeric plants)

FIG. 29 Regenerants obtained from cross C18-5. Plants 4 and 6 are white, while plant 10 is totally blue. The rest of the plants have many blue spots, in some cases very small.

FIG. 30-I PCR analysis of some of the plants regenerated from cross C18-5. Expected results in white (“A”), blue (“B”), and spotted plants (“C”).

FIG. 30-II PCR analysis of some of the plants regenerated from cross C18-5. PCR results of six plants, before (Panel “A”) and after (Panel “B”) digestion of the fragment with I-SceI

FIG. 31-II Southern blot of several the plants regenerated from cross C18-5, hybridized with a GUS probe. Schematic drawing for expected results with and without ICHR.
Upper Panel (“A”); genomic DNA digested with EcoRI+NotI.
Lower Panel (“B”): genomic DNA digested with BamHI+PvuI.

FIG. 31-II Southern blot of several the plants regenerated from cross C18-5, hybridized with a GUS probe. Southern results of six regenerated plants and a wild type control.
Left (Panel A): genomic DNA digested with EcoRI+NotI.
Right (Panel B): genomic DNA digested with BamHI+PvuI.

FIG. 32 Northern blot analysis of some of the plants regenerated from cross C18-5.
A: Ethidium bromide stained agarose gel to be blotted (10 μg total RNA)
B: Northern blot, probed with GUS probe
Note that due to the recombination event, the transcript produced by plant number 10 is shorter than the transcript produced by any of the other plants

FIG. 33: A: Principle of Southern hybridisation to distinguish between recombination cassette before (A-1) and after (A-2) homologous recombination (HR) occurred.
B: Southern blot of leaf material from F2 plants originating from crosses between Arabidopsis plants harbouring a single copy of the T-DNA from the construct to monitor ICHR (pCB583-40) and Arabidopsis plants harbouring PcUbi::I-SceI (JB010cqz). DNA was extracted from plant leaves, digested with SacI, separated on an agarose gel, transferred onto nylon membrane and hybridised with a radioactive labelled GUS probe. The result was analysed with the help of a phosphoimager.

FIG. 34. Leaves from plants (A/B) after histochemical GUS staining, which originated from crosses of I-SceI expressing plants and plants containing the pGU-Sce-US reporter construct. The only difference between these two plants is that in plant B I-SceI expression is under control of PcUbi promoter, while I-SceI in plant A is under control of 35S promoter.

### EXAMPLES

### General Methods

The chemical synthesis of oligonucleotides can be effected for example in the known manner using the phosphoamidite method (Voet, Voet, 2nd edition, Wiley Press New York, pages 896-897). The cloning steps carried out for the purposes of the present invention, such as, for example, restriction cleavages, agarose gel electrophoresis, purification of DNA fragments, the transfer of nucleic acids to nitrocellulose and nylon membranes, the linkage of DNA fragments, the transformation of E. coli cells, bacterial cultures, the propagation of phages and the sequence analysis of recombinant DNA are carried out as described by Sambrook et al. (1989) Cold Spring Harbor Laboratory Press; ISBN 0-87969-309-6. Recombinant DNA molecules were sequenced using an ALF Express laser fluorescence DNA sequencer (Pharmacia, Upsala, Sweden) following the method of Sanger (Sanger et al., Proc. Natl. Acad. Sci. USA 74 (1977), 5463-5467).

### Example 1

### Plant Transformation

### Example 1a

### Transformation of Arabidopsis thaliana

A. thaliana plants were grown in soil until they flowered. Agrobacterium tumefaciens (strain C58C1 (pMP90)) transformed with the construct of interest was grown in 500 mL in liquid YEB medium (5 g/L Beef extract, 1 g/L Yeast Extract (Duchefa), 5 g/L Peptone (Duchefa), 5 g/L sucrose (Duchefa), 0.49 g/L MgSO₄ (Merck)) until the culture reached an OD₆₀₀ 0.8-1.0. The bacterial cells were harvested by centrifugation (15 minutes, 5,000 rpm) and resuspended in 500 mL infiltration solution (5% sucrose, 0.05% SILWET L-77 (distributed by Lehle seeds, Cat. No. VIS-02)). Flowering plants were dipped for 10-20 seconds into the Agrobacterium solution. Afterwards the plants were kept in the greenhouse until seeds could be harvested. Transgenic seeds were selected by plating surface sterilized seeds on growth medium A (4.4 g/L MS salts (Sigma-Aldrich), 0.5 g/L MES (Duchefa); 8 g/L Plant Agar (Duchefa)) supplemented with 50 mg/L kanamycin for plants carrying the nptII resistance marker, 100 nM Bimazethapyr for plants carrying a mutated AHAS gene and 10 mg/L Phosphinotricin for plants carrying the pat gene, respectively. Surviving plants were transferred to soil and grown in the greenhouse.

### Example 1b

### Agrobacterium-Mediated Transformation of Brassica napus

Agrobacterium tumefaciens strain GV3101 transformed with the plasmid of interest was grown in 50 mL YEB medium (see Example 4a) at 28° C. overnight. The Agrobacterium solution is mixed with liquid co-cultivation medium (double concentrated MSB5 salts (Duchefa), 30 g/L sucrose (Duchefa), 3.75 mg/l BAP (6-benzylamino purine, Duchefa), 0.5 g/l MES (Duchefa), 0.5 mg/l GA3 (Gibberellic Acid, Duchefa); pH5.2) until OD₆₀₀ of 0.5 is reached. Petiols of 4 days old seedlings of Brassica napus cv. Westar grown on growth medium B (MSB5 salts (Duchefa), 3% sucrose (Duchefa), 0.8% oxoidagar (Oxoid GmbH); pH 5.8) are cut. Petiols are dipped for 2-3 seconds in the Agrobacterium solution and afterwards put into solid medium for co-cultivation (co-cultivation medium supplemented with 1.6% Oxoidagar). The co-cultivation lasts 3 days (at 24° C. and ˜50 μMol/m²s light intensity). Afterwards petiols are transferred to co-cultivation medium supplemented with 18 mg/L kanamycin (Duchefa) and 300 mg/L Timetin (Duchefa) and incubated for four weeks at 24° C. This step is repeated until shoots appear. Shoots are transferred to A6 medium (MS salts (Sigma Aldrich), 20 g/L sucrose, 100 mg/L myo-inositol (Duchefa), 40 mg/L adenine sulfate (Sigma Aldrich), 500 mg/L MES, 0.0025 mg/L BAP (Sigma), 5 g/L oxoidagar (Oxoid GmbH), 150 mg/L timetin (Duchefa), 15 mg/L kanamycin (Sigma), 0.1 mg/L IBA (indol butyric acid, Duchefa); pH 5.8) until they elongated. Elongated shoots are cultivated in A7 medium (A6 medium without BAP) for rooting. Rooted plants are transferred to soil and grown in the greenhouse.

### Example 2

### Constructs Harbouring Sequence Specific DNA-Endonuclease Expression Cassettes

### Example 2a

### Basic Construct

In this example we present the general outline of a binary vector, named “Construct I” suitable for plant transformation. This general outline of the binary vector comprises a T-DNA with a nos-promoter::nptII::nos-terminator cassette, which confers kanamycin resistance when integrated into the plant genome. SEQ ID NO: 1 shows a sequence stretch of “NNNNNNNNNN”. This is meant to be a placeholder for different expression cassettes for the sequence specific DNA-endonuclease. The sequence of the latter is given in the following examples.

### Example 2b

### Comparison Constructs

2b.1 Ovule Primordia, Egg Cell, Zygote and Early Embryo-Specific Promoter Fused to Endonuclease I-SceI

In Plant Phys. 127: 803-816 (2001) Hecht et al. described the promoter of the Arabidopsis gene AtSERK1 (Somatic Embryogenesis Receptor-Like Kinase 1; AtIG71830). The respective promoter fragment was fused to I-SceI. The resulting plasmid was called pCB603-100. The sequence of the construct is identical to the sequence of construct I, whereas the sequence “NNNNNNNNNN” was replaced by the sequence described by SEQ ID NO: 2.

2b.2 Zygote, Early Embryo and Meristematic Cells Specific Promoter Fused to Endonuclease I-SceI

In Plant Cell 6: 1763-1774 (1994) Ferreira et al. described the promoter of the Arabidopsis gene Atcyc1A (Cyclin cyc1 gene, type cyclin B; At4g37490). The respective promoter fragment was fused to I-SceI. The resulting plasmid was called pCB622-3. The sequence of the construct is identical to the sequence of construct 1, whereas the sequence “NNNNNNNNNN” was replaced by the sequence described by SEQ ID NO: 3.

2b.3 Shoot and Flower Meristems Specific Promoter Fused to Endonuclease I-SceI

The promoter of gene erecta (Acc. No. D83257) from Arabidopsis was described to be active in meristematic cells (Yokoyama et al., 1998, Plant J. 15: 301-310). The erecta promoter was fused to I-SceI resulting in plasmid pCB653-37. The sequence of the construct is identical to the sequence of construct I, whereas the sequence “NNNNNNNNNN” was replaced by the sequence described by SEQ ID NO: 4.

2b.4 Pollen-Specific Promoter Fused to Endonuclease I-SceI

The promoter of the potato invGF gene is active in pollen of potato (Plant Mol. Biol., 1999, 41: 741-751; EMBL Acc No. AJ133765) and rapeseed. The respective promoter fragment has fused to I-SceI. The resulting plasmid was called pCB652-124. The sequence of the construct is identical to the sequence of construct 1, whereas the sequence “NNNNNNNNNN” is replaced by the sequence described by SEQ ID NO: 5.

2b.5 Constitutive sTPT Promoter Fused to Endonuclease I-SceI

The sTPT promoter from Arabidopsis (i.e. TPT promoter truncated version, WO 03/006660; SEQ ID NO: 27 cited therein) is comparable to the well known 35S promoter. The sTPT promoter was fused to I-SceI. The sequence of the construct pCB632-17 is identical to the sequence of construct 1, whereas the sequence “NNNNNNNNNN” is replaced by the sequence described by SEQ ID NO: 6.

2b.6 Constitutive CaMV 35S Promoter Fused to the Endonuclease I-SceI

The 35S promoter from Cauliflower mosaic virus (CaMV) is a strong, well known promoter. The 35S promoter was fused to I-SceI. The resulting construct was named pRS8 (SEQ ID NO: 13).

### Example 2c

### Parsley Promoter Fused to Endonuclease I-SceI

The PcUbi promoter from parsley (WO 03/102198) is a strong constitutive promoter. The PcUbi promoter was fused to I-SceI. This resulted in two constructs named JB010qcz (SEQ ID NO: 11) and pCB666-3 (SEQ ID NO: 9).

### Example 3

### Constructs Used to Monitor Intrachromosomal Homologous Recombination (ICHR) and Marker Excision

### Example 3a

### Construct to Monitor ICHR by Restoring GUS Activity

In construct pCB583-40 (SEQ ID NO: 12) the T-DNA comprises the 35S CaMV constitutive promoter, a partial uidA (GUS) gene (called “GU”), an I-SceI recognition sequence and another partial uidA gene (called “US”) as well as ocs terminator.

The partially overlapping halves of the GUS gene (GU and US) are non-functional, but as a result of ICHR a functional GUS gene will be restored. This can be monitored by hostochemical GUS staining (Jefferson 1985)

### Example 3b

### Construct to Demonstrate Marker Excision

In construct pCB657-41 (SEQ ID NO: 10) the nit1P::nptII selectable marker expression cassette is surrounded by recognition sites for I-SceI and I-CreI and a direct repeat of the 35S terminator sequence. The 35S terminator sequence also functions as terminator for the nit1P::nptII expression cassette. The GUS gene functions as a reporter gene under control of the constitutive sTPT promoter (see above). The GUS gene as well as the sTPT promoter can easily be replaced by any other promoter::gene of interest cassette.

Thus, upon induction of ICHR by double-strand breaks, recombination between the duplicated terminator sequences may occur and lead to the loss of the nitP::nptII selectable marker expression cassette (i.e. marker excision), while the GUS expression cassette stays in the genome.

### Example 4

### Transformation of Sequence-Specific DNA Endonuclease Encoding Constructs into Arabidopsis thaliana

Plasmids pCB603-100, pCB622-3, pCB653-37, pCB632-17, pCB652-124, pRS8, JB010qcz were transformed into Arabidopsis according to the protocol described in Example 1a. Selected transgenic lines (T1 generation) were grown in the greenhouse and some flowers were used for crossings (see below).

### Example 5

### Transformation of Constructs to Monitor Marker Excision into Arabidopsis thaliana

Plasmid pCB583-40 as well as pCB657-41 were transformed into Arabidopsis according to the protocol described in Example 1a. Selected transgenic lines (T1 generation) had been grown in the greenhouse and seeds had been harvested. T2 seeds had been grown in vitro on growth medium A (see Example 1a) supplemented with the respective selective agent (10 mg/L Phosphinotricin and 50 mg/L kanamycin, respectively). Individual, resistant plants from lines showing a 3:1 segregation have been transferred to soil and grown in the greenhouse.

### Example 6

### Transformation of Sequence-Specific DNA Endonuclease Encoding Constructs into Brassica napus

Plasmid pCB632-17, pRS8 and pCB666-3 are transformed into rapeseed according to the protocol described in Example 1b. Selected transgenic lines (T1 generation) are grown in the greenhouse.

### Example 7

### Transformation of Constructs to Monitor Marker Excision into Brassica napus

Plasmid pCB657-41 is transformed into rapeseed according to the protocol described in Example 1b. Selected transgenic lines (T1 generation) are grown in the greenhouse. For comparison a construct pGU-Sce-US (identical to pCB583-40, but with nptII instead of pat selectable marker) has been transformed into rapeseed according to the protocol described in Example 1b. Selected transgenic lines (T1 generation) are grown in the greenhouse.

### Example 8

### Induction of ICHR by Crossing Sequence-Specific DNA Endonuclease Expressing Lines and Lines Harboring Constructs to Monitor ICHR and Marker Excision

### Example 8a

### Monitoring ICHR in Arabidopsis

Transgenic lines of Arabidopsis harboring the T-DNA of construct pCB583-40 have been crossed with lines of Arabidopsis harboring the T-DNA of constructs pCB603-100, pCB622-3, pCB653-37, pCB632-17, pCB652-124, pRS8, JB010qcz, respectively. F1 seeds of the crosses have been harvested. The seeds have been surface sterilized and grown on medium A supplemented with the respective antibiotics and/or herbicides. 3-4 old seedlings have been harvested and were used for histochemical GUS staining. The result is summarized in Table 2 and illustrated in FIG. 27 for one particular example.

TABLE 2
Amount of blue areas as an indicator of tissues/parts
of tissues in which ICHR occurred in crosses

| Promoter driving I-Scel | | Relative amount of tissues |
|---|---|---|
| expression | Construct | in which ICHR occurred |
| Control (no I-Scel crossed | / | −(spontaneous frequency of |
| into reporter lines) | | ICHR in seedlings) |
| erecta | pCB653-37 | − |
| AtCyc1 | pCB622-3 | + |
| AtSERK1 | pCB603-100 | − |
| invGF | pCB652-124 | − |
| 35S | pRS8 | +++ |
| sTPT | pCB666-3 | ++++ |
| PcUbi | JB010cqz | ++++++++ |

Some seedlings have been transferred to soil and seeds have been harvested. 3-4 weeks old F2 seedlings have been analysed by histochemical GUS staining. Only in crosses harbouring the GU-US reporter (pCB583-40) as well as the PcUbi::I-SceI construct (JB010cqz) completely blue plants have been detected. This indicates that almost all or all cells of the respective plants harboured the ICHR event. This was further confirmed by Southern hybridisation (FIG. 33). While F2 plants 6, 3 and 13 from crosses K165-10, K170-10 and K170-10, respectively, show only the recombined event, other plants are chimeric and show the hybridisation band indicative for both, recombined and non-recombined events. This correlated with the histochmical GUS staining: K165-10-6, K170-10-3 and K170-10-13 were completely blue after histochemical GUS staining, while K170-4-3, K161-6-3, K161-6-7, for example, showed only blue sectors and blue spots. The conclusion that completely blue (recombined) F2 plants have been obtained was further confirmed by analysing the F3 descendants of the respective F2 plants. As expected, F3 plants were completely blue or completely white (as a result of segregation of the GUS gene). F3 descendants of K170-10-3, for example, were completely blue or completely white after histochemical GUS staining. Completely blue and completely white F3 plants appeared in a 3:1 ratio, indicating that the respective F2 plant was heterozygous for the GUS gene and the adjacent recombination cassette. F3 descendants of K170-10-13, for example, were all completely blue indicating that the respective F2 plant was homozygous for the GUS gene and the adjacent recombination cassette. Thus expression of I-SceI via parsley ubiquitin (PcUbi) promoter was so efficient that recombination had been induced on the maternal and paternal chromosome.

Therefore, the cassette PcUbi::I-SceI appears to be suitable for obtaining fully marker-free plants in the F2 of a respective cross.

### Example 8b

### Demonstrating Marker Excision in Arabidopsis

Transgenic lines of Arabidopsis harbouring a single integration of the T-DNA of construct pCB657-41 are crossed with lines of Arabidopsis harbouring the T-DNA of construct JB010qcz. F1 seeds of the crosses are harvested. The seeds are surface sterilized and grown on medium A supplemented with 100 nM Bimazethapyr. After transfer to soil F2 seeds are harvested. The F2 seeds are sawn and analysed by PCR. Plants which do not show a PCR fragment for the nptII selectable marker, but show a PCR fragment for the GUS gene still present in the T-DNA of construct pCB657-41 after the ICHR event occurred, are free of the selectable marker encoded in the T-DNA of construct pCB657-41 due to the I-SceI induced induction of ICHR. This is confirmed by Southern Analysis. In the F3 generation plants will be selected, which lost the T-DNA of construct JB010qcz due to segregation. The respective plants are free of any selectable marker and do not obtain I-SceI, but only the gene of interest (i.e. GUS in this particular example).

The following Table 3 exemplifies such analysis for a particular cross (CR-CB613) between Arabidopsis plants harbouring a single integration of the T-DNA of construct pCB657-41 and lines of Arabidopsis harbouring the T-DNA of construct JB010qcz (PcUbi::I-SceI). Shown are results of a histochemical GUS staining of 30 F2 plants (named C24-CR-CB1055-PL-1 to 30) originating from line 3 of cross CR-CB613. The histochemical GUS staining is indicative for the presence of the GUS gene from the T-DNA of construct pCB657-41. A PCR to detect the nptII gene was conducted on GUS positive plants. The absence of nptII indicates that marker excision occurred and the T-DNA of pCB657-41 integrated into the respective Arabidopsis plants was recombined (nd—not determined). C24-CR-CB1055-PL-6, C24-CR-CB1055-PL-7, C24-CR-CB1055-PL-14 and C24-CR-CB1055-PL-20 may have lost the nitP::nptII selectable marker cassette from the T-DNA of pCB657-41 due to ICHR induced by I-SceI expressed under control of PcUbi promoter.

TABLE 3
Analysis of F2 Arabidopsis plants in order
to identify selectable marker-free plants

| Plant name | GUS staining | PCR nptII |
|---|---|---|
| C24-CR-CB1055-PL-1 | − | nd |
| C24-CR-CB1055-PL-2 | + | + |
| C24-CR-CB1055-PL-3 | + | + |
| C24-CR-CB1055-PL-4 | + | + |
| C24-CR-CB1055-PL-5 | + | + |
| C24-CR-CB1055-PL-6 | + | − |
| C24-CR-CB1055-PL-7 | + | − |
| C24-CR-CB1055-PL-8 | + | + |
| C24-CR-CB1055-PL-9 | − | − |
| C24-CR-CB1055-PL-10 | − | nd |
| C24-CR-CB1055-PL-11 | + | + |
| C24-CR-CB1055-PL-12 | + | + |
| C24-CR-CB1055-PL-13 | + | + |
| C24-CR-CB1055-PL-14 | + | − |
| C24-CR-CB1055-PL-15 | + | + |
| C24-CR-CB1055-PL-16 | + | + |
| C24-CR-CB1055-PL-17 | + | + |
| C24-CR-CB1055-PL-18 | + | + |
| C24-CR-CB1055-PL-19 | + | + |
| C24-CR-CB1055-PL-20 | + | − |
| C24-CR-CB1055-PL-21 | + | + |
| C24-CR-CB1055-PL-22 | + | + |
| G24-CR-CB1055-PL-23 | + | + |
| C24-CR-CB1055-PL-24 | − | nd |
| C24-CR-CB1055-PL-25 | − | nd |
| C24-CR-CB1055-PL-26 | + | + |
| C24-CR-CB1055-PL-27 | + | + |
| C24-CR-CB1055-PL-28 | + | + |
| C24-CR-CB1055-PL-29 | − | nd |
| C24-CR-CB1055-PL-30 | − | nd |

### Example 8c

### Monitoring ICHR in Rapeseed

Intrachromosomal homologous recombination (ICHR) is the mechanism underlying the marker excision concept described in this invention. This comparison example illustrates the results obtained when using ICHR in combination with the expression of an endonuclease under the control of a constitutive promoter. The example uses two constructs: pRS8 and pGU-sce-US, both binary vectors with nptII as a selection marker.

The T-DNA from pRS8 (SEQ ID NO.: 13), which is defined by its left and right borders (LB and RB, respectively), contains the following elements (from LB to RB): nptII expression cassette (nos promoter—nptII coding sequence—nos terminator); I-SceI expression cassette (CaMV 35S promoter—I-SceI coding sequence—ocs terminator). The T-DNA described is located on a plasmid (pSUN derivative) that contains origins for the propagation in E. coli as well as in Agrobacterium and an aadA expression cassette (conferring spectinomycin and streptomycin resistance) to select for transgenic bacteria cells.

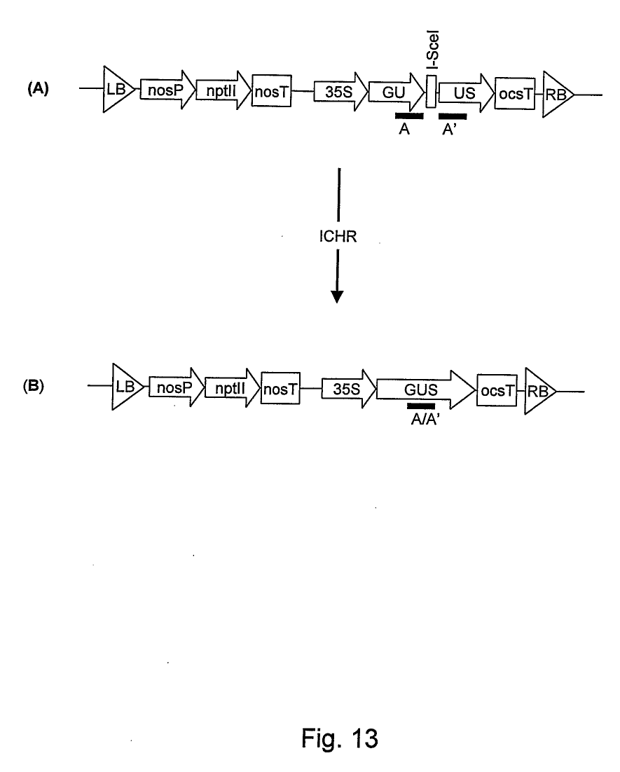

The T-DNA from pGU-sce-US (FIG. 13), which is defined by its left and right borders (LB and RB, respectively), contains the following elements (from LB to RB): nptII expression cassette (nos promoter—nptII coding sequence—nos terminator); constitutive promoter—partial uidA (GUS) gene (called “GU”)—I-SceI recognition sequence—partial uidA gene (called “US”)—ocs terminator. The partially overlapping halves of the GUS gene (GU and US) are non-functional, but as a result of ICHR a functional GUS gene will be restored. The T-DNA described is located on a plasmid (pSUN derivative) that contains origins for the propagation in E. coli as well as in Agrobacterium and an aadA expression cassette (conferring spectinomycin and streptomycin resistance) to select for transgenic bacteria cells.

The constructs pRS8 and pGU-sce-US, respectively, were separately introduced into B. napus via Agrobacterium-mediated transformation using the procedure described in Example 1b. Transgenic lines containing the separate constructs were selected in kanamycin-containing media and confirmed by molecular analysis (genomic PCR and genomic Southern blots). Several independent lines containing the pRS8 T-DNA and the pGU-sce-US T-DNA, respectively, were isolated. These lines contained between 1 and 5 copies of the respective T-DNA, as determined by Southern blot.

TO pRS8 and pGU-sce-US transgenic rapeseed plants (heterozygous for the respective transgenes) were crossed. F1 lines of these crosses were analyzed by genomic PCR and Southern blot in order to identify plants containing both transgenes. These plants were used for histochemical GUS staining and compared to siblings containing only the GU-sce-US transgene in order to determine the effect of double-strand breaks on ICHR.

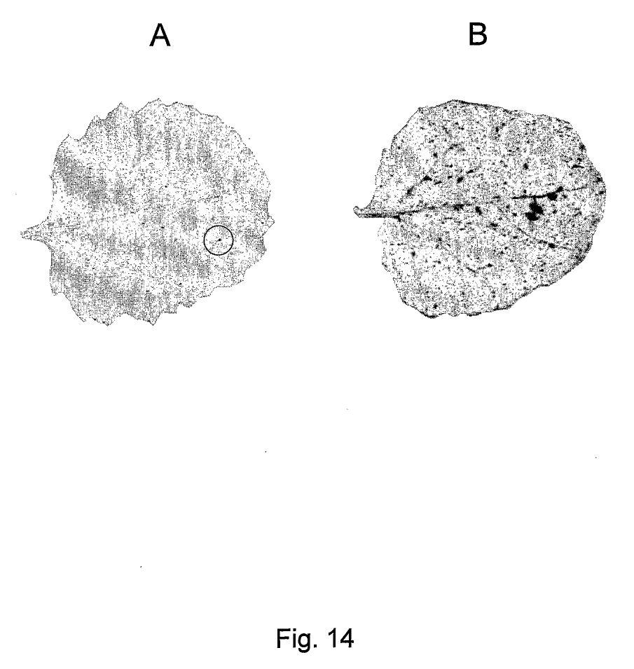

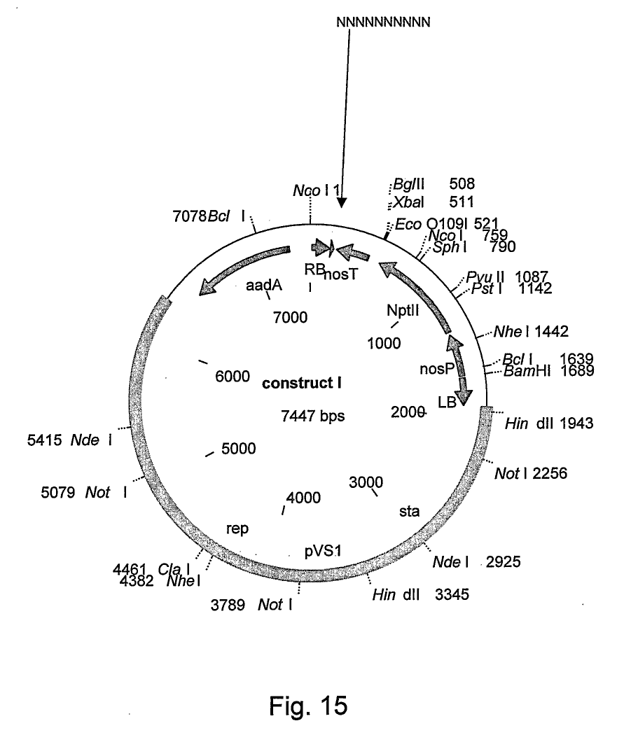

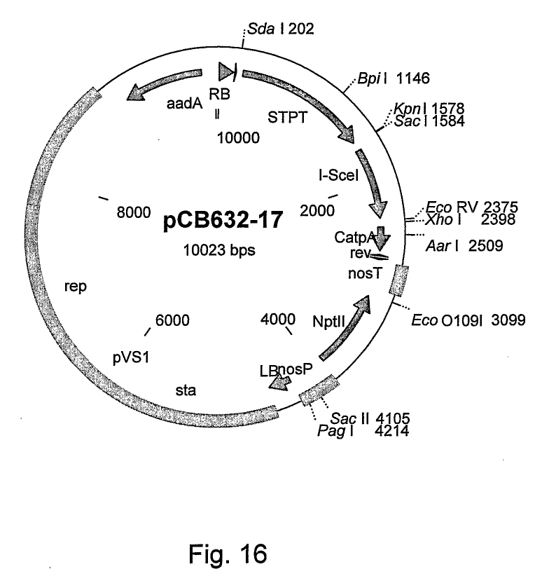

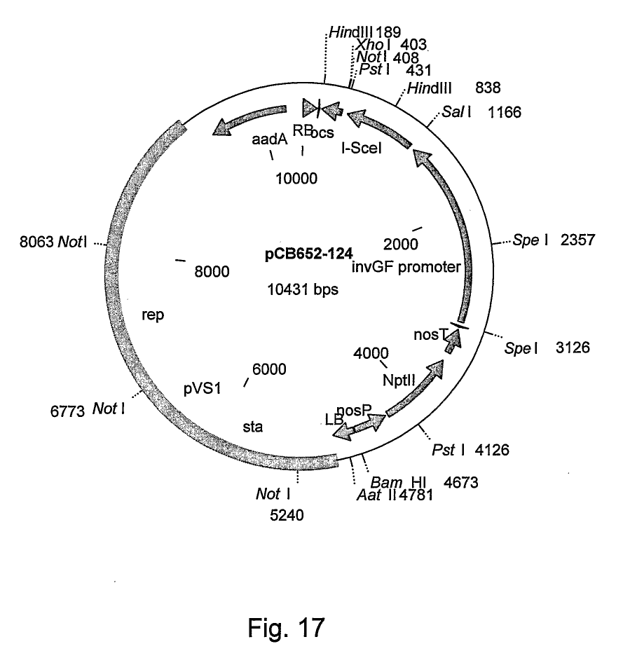

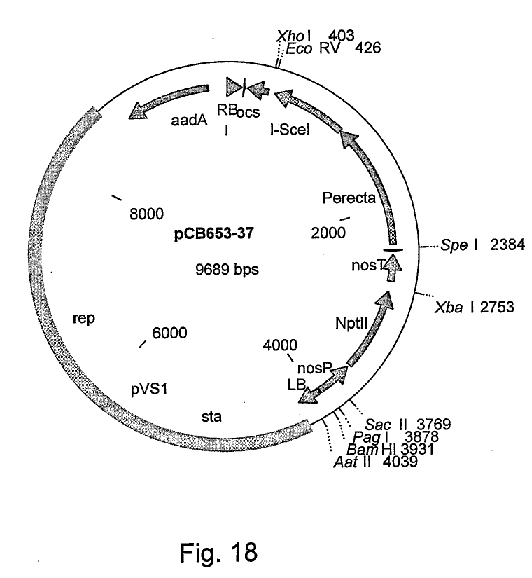

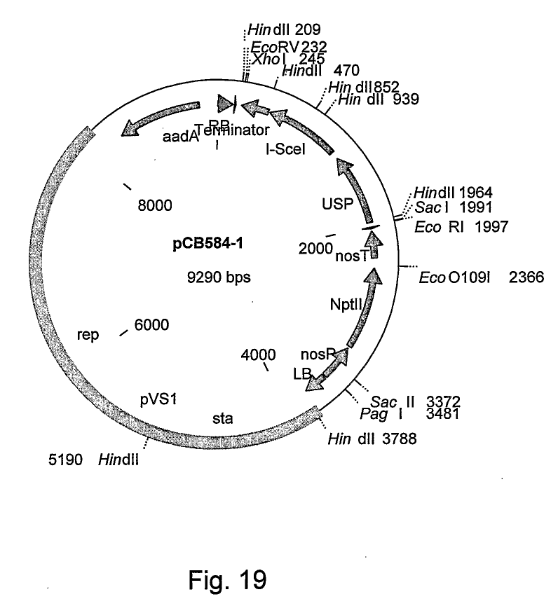

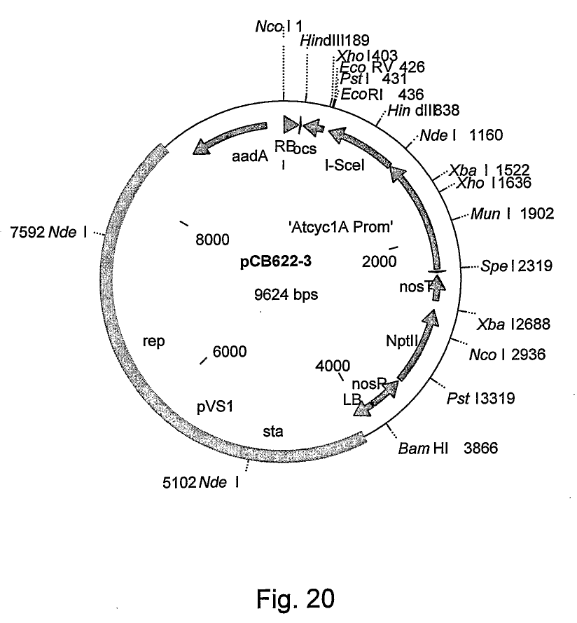

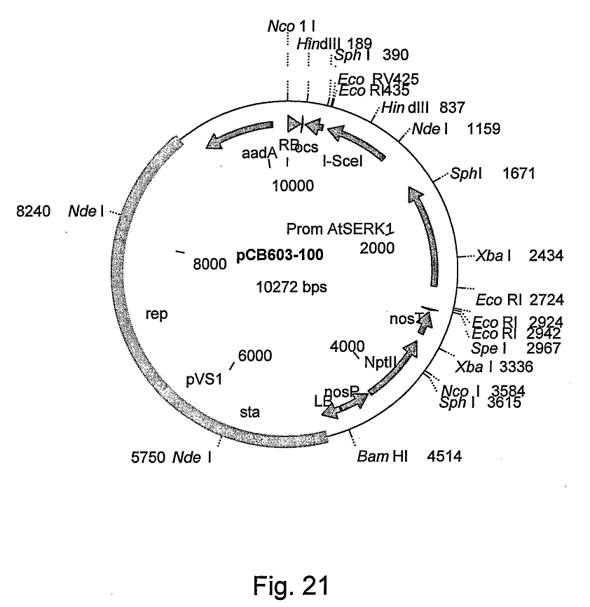

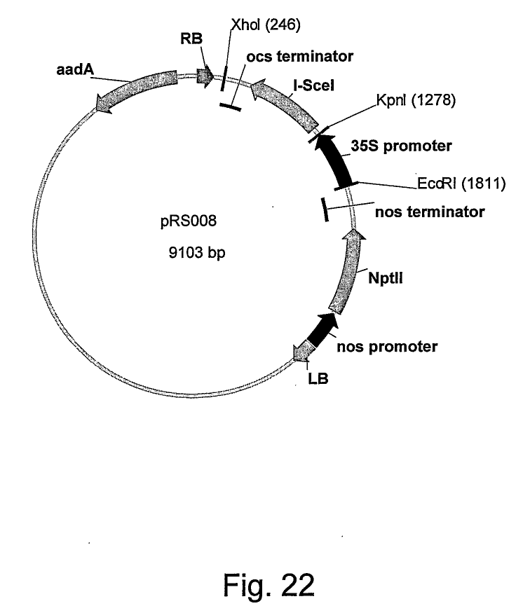

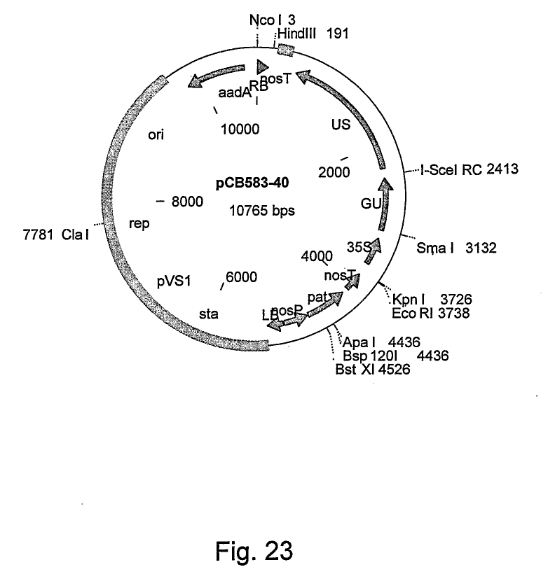

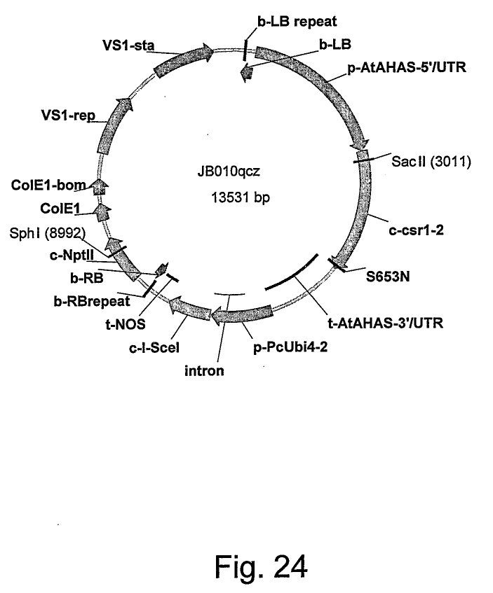

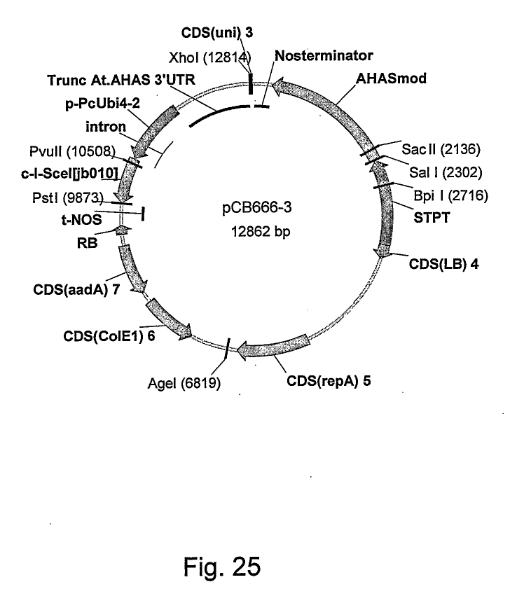

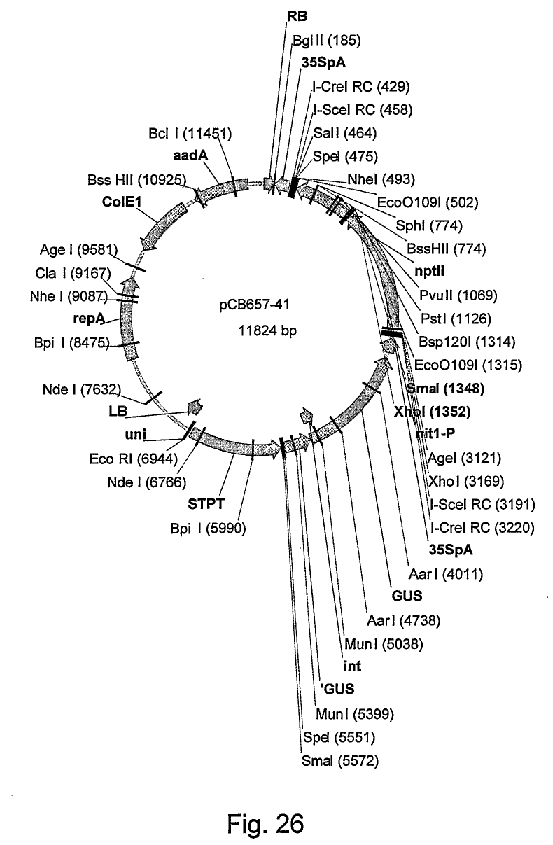

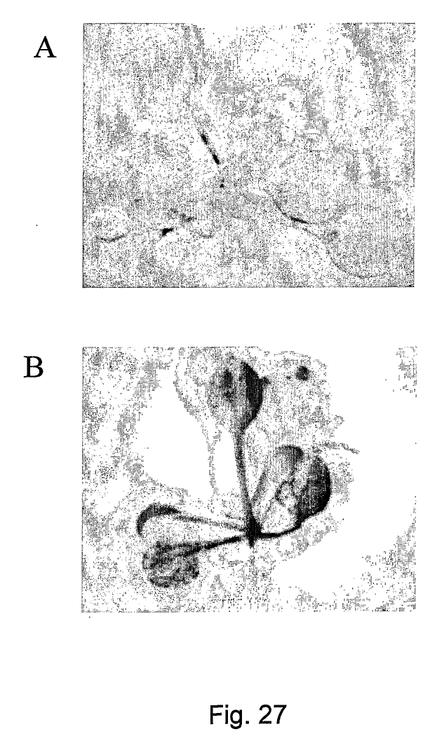

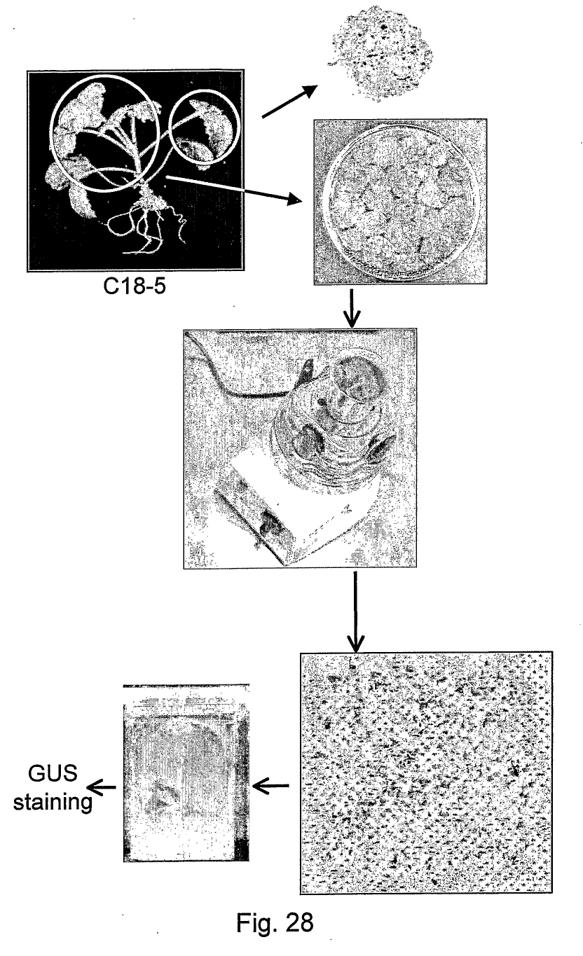

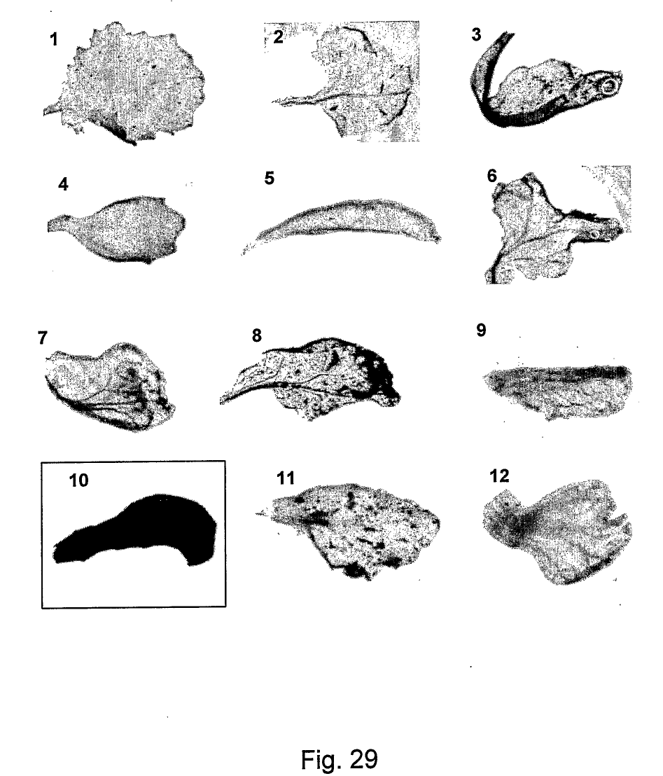

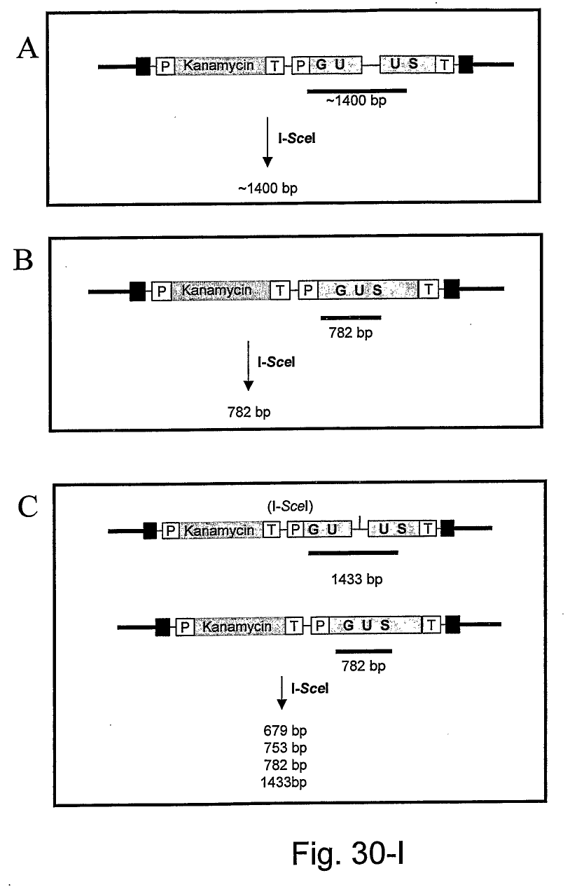

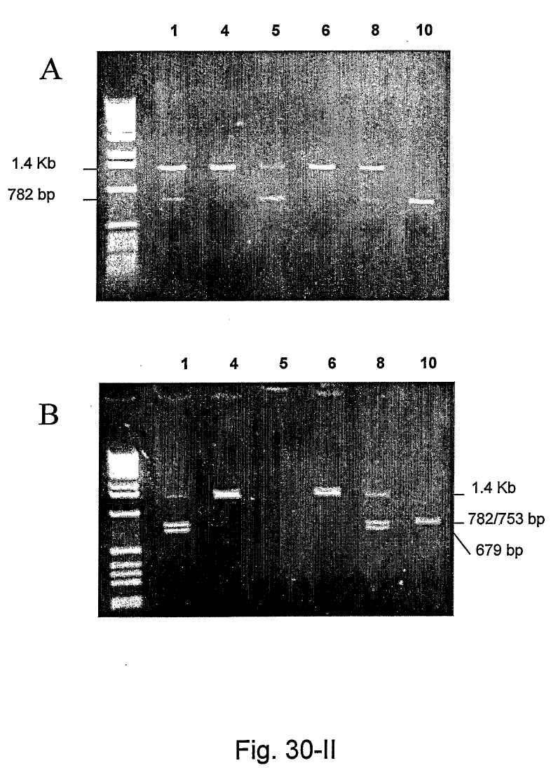

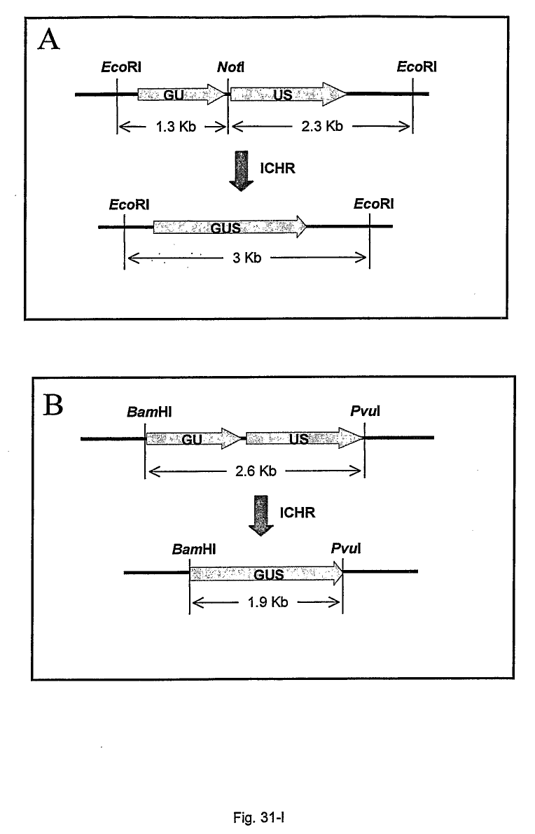

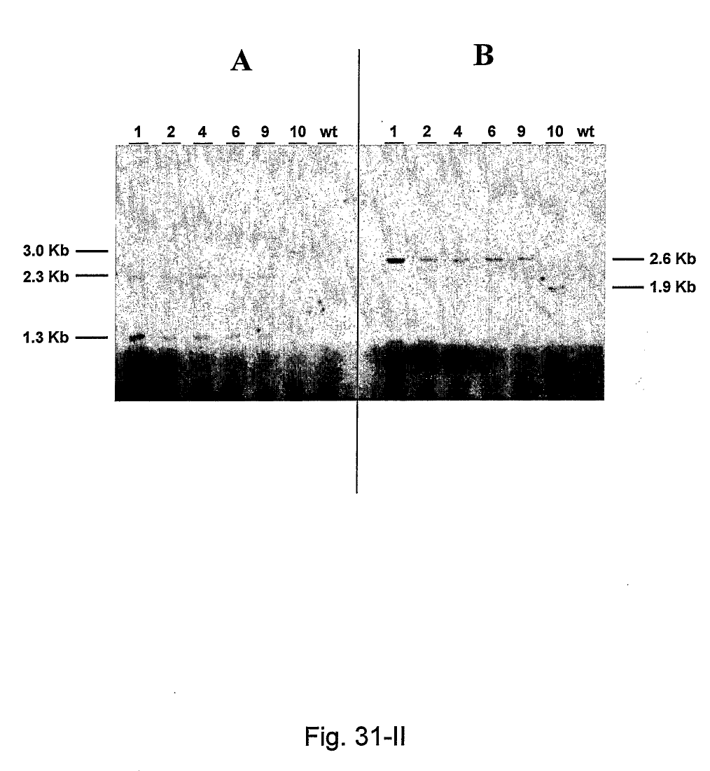

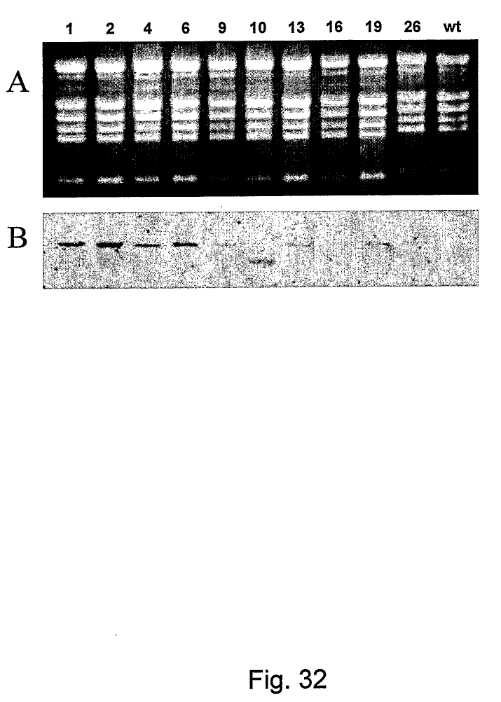

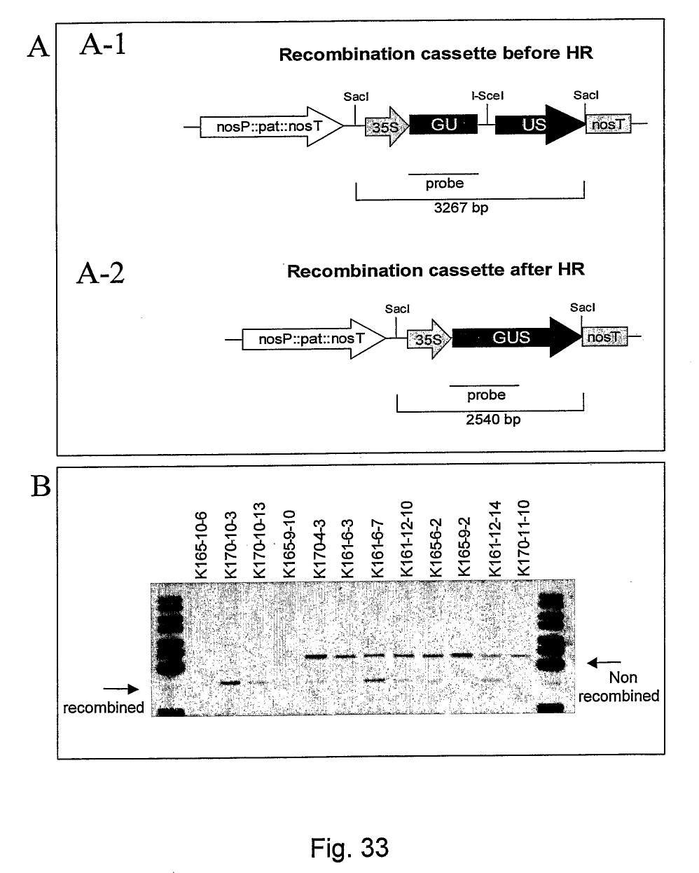

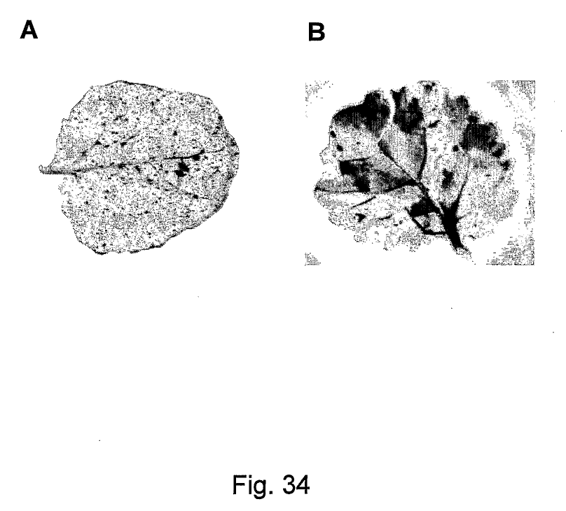

Results: A dramatic increase in the frequency of ICHR due to the expression of the I-SceI can be observed, as shown by the number of blue spots per leaf, which are originated via ICHR between the duplicated parts of the GUS gene present in the GU-sce-US construct. Although the introduction of double-strand breaks by the expression of the I-SceI nuclease caused a very significant increase on the frequency of ICHR, so far all F1 plants analyzed were chimeric and no F1 plant having a entire blue staining was observed (FIG. 14).

Non-chimeric plants (exhibiting a complete blue staining) can be obtained by regenerating new plants from the marker free (or blue in this example) sectors on the chimeric plants (e.g., by inducing somatic embryogenesis and/or organogenesis/shoot generation). (The tissue culture process is summarized in FIG. 28).

One particular pRS8 X pGU-sce-US cross, called C18, produced 9 F₁ plants that were called C18-1, C18-2, C18-3, C18-4, C18-5, C18-6, C18-7, C18-8, and C18-9, respectively. Three of these plants had many blues spots (C18-5, C18-7 and C18-9). Around sterile F₂ seeds from plant C18-5 were germinated on MSB5 medium (4.4 g/l MS medium with B5 vitamins, 0.5 g/l MES, 3% sucrose, 0.8% oxoid agar; pH 5.8) and incubated at 21° C., 16 h light (40-50 μE/m²s)/8 h dark for three weeks. The plants were then cut approx. 1 cm below the cotyledons and the top was transferred to fresh MSB5 medium for ˜3 more weeks in the same conditions. When the leaves were 1-3 cm, one leaf per plant was used for GUS staining, and only the plants showing many spots per leaf were kept. The rest of the leaves of the positive plants were harvested, pooled, immersed in disruption medium (4.4 g/l MS medium with B5 vitamins, 0.5 g/l MES, 13% sucrose, 3,75 mg/l BAP, pH 5.8) and disrupted using a waring blender (3-5 pulses of 3-5 seconds). 100 mg aliquots of the leaf slurry obtained were plated on osmotic rafts over liquid regeneration medium (4.4 g/l MS medium with B5 vitamins, 0.5 g/l MES, 3% sucrose, 3 mg/l AgNO₃, 5 mg/l BAP, 5 mg/l NAA; pH 5.8), and subcultured every ˜10 days until calli (first) and shoots (later) appeared. The shoot-forming explants were transferred to shoot development medium (4.4 g/l MS medium with B5 vitamins, 0.5 g/l MES, 1% sucrose, 3 mg/l BAP, 1 mg/l zeatin; pH 5.8), and subcultured every ˜10 days until shoots reached a size of 1.5-2 cm. Then they were transferred to Magenta boxes with shoot elongation medium (4.4 g/l MS medium with B5 vitamins, 0.5 g/l MES, 1% sucrose, 0.6% oxoid agar; pH 5.8) and subcultured every 15-20 days. When the shoots were big enough, all callus tissue was discarded by cutting and the rest of the explant was transferred to fresh medium for rooting. In total we regenerated 25 plants (FIG. 29 shows regenerants 1 to 10). Of these, 20 had many spots, 3 were white (numbers 4, 6 and 26), and 2 were totally blue (numbers 10 and 15). The blue plants were regenerated from cells in which I-SceI cut between “GU” and “US” and the double strand break was repaired by intrachromosomal homologous recombination. The white plants were regenerated from cells in which I-SceI cut between “GU” and “US” and the double strand break was repaired by non homologous end joining (illegitimate recombination). The plants with spots were regenerated from cells in which I-SceI did not cut (or it did cut and the break was repaired, restoring the I-SceI recognition sequence). This was confirmed by the molecular analysis of several of these plants (PCR, Southern blot, and Northern blot). We performed PCRs (FIG. 30-I/II) with primers that amplify a band of 1433 bp on the unrecombined GU-US substrate. If recombination has taken place, we expect a fragment of 782 bp, which is what we obtained with regenerated plant number 10 (full blue plant). The white plants should produce a band of ˜1400 bp, which is what we obtained with regenerated plants number 4 and 6 (white plants). The plants with spots must show both the 1433 and the 782 bp bands, and as expected regenerated plants number 1, 5 and 8 did so. These results were confirmed by digesting the PCR fragments with I-SceI. The enzyme only cut the 1433 bp band produced by the plants with spots, which are the only ones that still contained an I-SceI recognition sequence. In addition we performed a Southern blot analysis, digesting genomic DNA from regenerated plants with EcoRI+NotI or BamHI+PvuI (FIG. 31-I/II). With the first double digestion and using a GUS probe, we obtained two bands of 1.3 and 2.3 kb in all plants except number 10, which gave only one band of 3 Kb. With the second double digestion and the same probe, we obtained one band of 2.6 Kb in all plants except number 10, which gave one band of 1.9 Kb after. These results confirmed that plant number 10 was indeed totally blue (equivalent to full marker excision). In addition Northern blot analysis (FIG. 32) using a GUS probe showed that plant number 10 produced a transcript that was ˜750 bp shorter than the transcript produced by the other plants, as expected after ICHR. Taken together, the results showed in FIGS. 3, 4 and 5 unequivocally prove that we can obtain fully recombined plants (i.e. full blue plants) after regeneration from plants in which recombination was partial (i.e. plants with blue spots).

Rapeseed lines harbouring the T-DNA of pCB666-3, i.e. the I-SceI under control of the PcUbi promoter have been crossed to rapeseed plants harbouring the T-DNA of pGU-sce-US. FIG. 34 B shows a leaf of such a cross after histochemical GUS staining in comparison to a cross of RS8 (35S::I-SceI) and pGU-sce-US plants (FIG. 34 A). PcUbi driving I-SceI is especially good in creating big sectors in which recombination—as monitored by restoration of a functional GUS gene—occurs. As a result when PcUbi promoter was used, a much bigger area of the leaf comprises cells with the recombined event. In addition, these results demonstrate that PcUbi promoter driving I-SceI expression is superior over other promoter::I-SceI combinations including the gold-standard 35S promoter not only in the model Arabidopsis but also in other species such as the important crop rapeseed.

The frequency of obtaining completely blue plants with the method described above is much higher when the pGU-sce-US harbouring lines are crossed with rapeseed lines harbouring the T-DNA of pCB666-3, i.e. the I-SceI under control of the PcUbi. This demonstrates that the PcUbi promoter is much better suited for the purpose described in this invention.

### Example 8d

### Demonstrating Marker Excision in Rapeseed

T1 plants harboring the T-DNA of construct pCB657-41 are crossed with lines harboring the plasmids pCB666-3 and pCB632-17, respectively. Seeds of the crosses are harvested and are germinated. The F1 seedlings of the crosses are used for regenerating new, completely marker free plants (regarding the selectable marker encoded between the duplication of the 35S terminator in pCB657-41; the GUS gene in this construct in this particular example is not used as a selectable marker. The selectable marker present in the T-DNA comprising the I-SceI-T-DNA from construct pCB663-3 and pCB632-17, respectively—is not intended to be deleted by this process. This marker is being segregated from the remaining GUS gene in the next generation; for the detailed protocol of regenerating new rapeseed plants from the F1 seedlings see above).

Alternatively to the regeneration protocol marker free plants are obtained by the following procedure. F2 plants are analyzed by PCR for the presence of I-SceI and the GUS reporter gene as well as for the absence of the nptII selectable marker cassette from the T-DNA of pCB657-41. In the F3 progeny then seedlings can be identified in which the T-DNA comprising I-SceI is segregated.

1. A method for producing a transgenic plant comprising:
i) crossing a first transgenic plant comprising in its genome a DNA construct comprising the following elements:
a1) at least one recognition sequence of at least 10 base pairs for the site-directed induction of DNA double-strand breaks by a sequence specific DNA-endonuclease, and
b1) a nucleic acid sequence to be excised,
wherein said elements a1) and b1) and optionally further elements are flanked by homology sequences A and A′, having sufficient length and sufficient homology in order to ensure homologous recombination between A and A′, and having an orientation which—upon recombination between A and A′—will lead to an excision of said elements a1) and b1), and
c1) at least one additional sequence conferring to said plant an agronomically valuable trait, wherein said sequence is not localized between the homology sequences A and A′ and would not be excised from the genome upon recombination between A and A′,
with a second transgenic plant comprising in its genome an expression cassette comprising
a2) a parsley ubiquitin promoter, and operably linked thereto
b2) a nucleic acid sequence coding for a sequence specific DNA-endonuclease having sequence specificity for said recognition sequence of the element a1),
ii) generating descendants (F1) following this crossing, and—optionally—sexually or asexually generating further descendants, and
iii) isolating descendants which have undergone recombination between the homology sequences A and A′ and which do not comprise in their genome said elements a1) and b1) but comprise sequence c1).

2. The method of claim 1, wherein the element b1) is an expression cassette for a marker sequence.

3. The method of claim 2, wherein the marker sequence is selected from the group consisting of negative selection marker, counter selection marker, positive selection marker, and reporter genes.

4. The method of claim 1, wherein the method further comprises steps of segregating the expression cassette for the endonuclease from the sequence c1) for the agronomically valuable trait and isolating plants comprising sequence c1) but not said expression cassette for the endonuclease.

5. The method of claim 1, wherein the parsley ubiquitin promoter comprises a sequence described by SEQ ID NO: 8 or 15 or a functional equivalent or functional equivalent fragment thereof.

6. The method of claim 1, wherein the homology sequences is are oriented in form of direct repeats, which are flanking elements a1) and b1) and optionally further elements.

7. The method of claim 1, wherein the sequence specific DNA-endonuclease is a homing endonuclease.

8. The method of claim 1, wherein the sequence specific DNA-endonuclease is a homing endonuclease selected from the group consisting of I-SceI, I-CpaI, I-CpaII, I-CreI and I-ChuI.

9. The method of claim 8, wherein the sequence encoding the endonuclease comprises an intron.

10. The method of claim 1, wherein said construct comprises two recognition sequences of element a1) which are localized between the homology sequences A and A′ and are flanking element b1) and optionally further elements in a way that cleavage at this the two recognition sequences excises said element b1).

11. The method of claim 1, wherein the homology sequences A and A′ are part of the expression cassette comprised in the DNA construct.

12. The method of claim 1, wherein the resulting plant is marker-free.

13. A transgenic expression cassette comprising a sequence coding for a sequence specific DNA-endonuclease operably linked to a parsely ubiquitin promoter.

14. The transgenic expression cassette of claim 13, wherein the sequence specific DNA-endonuclease is a homing endonuclease selected from the group consisting of I-Scel, I-CpaI, I-CpaII, I-Crei and I-ChuI.

15. The transgenic expression cassette of claim 14, wherein the sequence encoding the homing endonuclease comprises an intron.

16. The transgenic expression cassette of claim 13, wherein the parsley ubiquitin promoter comprises a sequence described by SEQ ID NO: 8 or 15 or a functional equivalent or functional equivalent fragment thereof.

17. A transgenic vector comprising the expression cassette of claim 13.

18. A transgenic cell or non-human organism comprising the expression cassette of claim 13.

19. A transgenic plant or plant cells comprising in the genome the expression cassette of claim 13.
